SEQUENCE LISTING (319 sequences)

SEQ ID NO: 1 - protein, 118 aa, Artificial Sequence
  12H10.G7-VH
EVQLQESGPELVKPGASVKMSCKASGYTFTRYVMHWVKQRPGQGLEWIGFINPYNDDTKYNEKFKGKATLTSDKSSSTAYMELSSLTSEDSAVYHCARWRQLGSLDSWGQGTTLTVSS

SEQ ID NO: 2 - protein, 111 aa, Artificial Sequence
  12H10.G7-VL
NIVLTQSPASLAVSLGQRATISCRASESVDTYGSSFVHWYQQKPGQPPKLLIYLASNLESGVPARFSGSGSRSDFTLTIDPVEADDAATYYCQQNNEEPWTFGGGTKLEIK

SEQ ID NO: 3 - protein, 249 aa, Artificial Sequence
  scFv of humanized 12H10.G7 GB87(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIK

SEQ ID NO: 4 - protein, 6 aa, Artificial Sequence
  12H10.G7-VH CDR2
NPYNDD

SEQ ID NO: 5 - protein, 9 aa, Artificial Sequence
  12H10.G7-VH CDR3
WRQLGSLDS

SEQ ID NO: 6 - protein, 15 aa, Artificial Sequence
  12H10.G7-VL CDR1
RASESVDTYGSSFVH

SEQ ID NO: 7 - protein, 7 aa, Artificial Sequence
  12H10.G7-VL CDR2
LASNLES

SEQ ID NO: 8 - protein, 9 aa, Artificial Sequence
  12H10.G7-VL CDR3
QQNNEEPWT

SEQ ID NO: 9 - protein, 118 aa, Artificial Sequence
  Humanized 12H10.G7-VH
QVQLVQSGAEVKKPGASVKVSCKASGYTFTRYVMHWVRQAPGQRLEWMGFINPYNDDTKYNEKFKGRVTITSDTSASTAYMELSSLRSEDTAVYHCARWRQLGSLDSWGQGTTVTVSS

SEQ ID NO: 10 - protein, 111 aa, Artificial Sequence
  Humanized 12H10.G7-VL
DIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGGGTKVEIK

SEQ ID NO: 11 - protein, 7 aa, Artificial Sequence
  Humanized 12H10.G7 GB87/GB95-VH CDR1
GYTFTRY

SEQ ID NO: 12 - protein, 249 aa, Artificial Sequence
  scFv of humanized 12H10.G7 GB95(VL-VH)
DIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSS

SEQ ID NO: 13 - protein, 118 aa, Artificial Sequence
  Humanized 12H10.G7 GB88/GB96-VH
QVQLVQSGAEVKKPGASVKVSCKASGYTFTRYVMHWVRQAPGQRLEWMGFINPYNDDTKYNEKFKGRVTITRDTSASTAYMELSSLRSEDTAVYHCARWRQLGSLDSWGQGTTVTVSS

SEQ ID NO: 14 - protein, 118 aa, Artificial Sequence
  Humanized 11F4.B9
QVQLVQSGAEVKKPGASVKVSCKASGYSFTGYYIHWVRQGPGQGLEWMGEIIPSTGSTIYAQKFQGRVTMTRDTSTSTVYMELSSLRSEDTAVYYCERWGDYYGRDYWGQGTLVTVSS

SEQ ID NO: 15 - protein, 249 aa, Artificial Sequence
  scFv of humanized 12H10.G7 GB88(VH-VL)
QVQLVQSGAEVKKPGASVKVSCKASGYTFTRYVMHWVRQAPGQCLEWMGFINPYNDDTKYNEKFKGRVTITRDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIK

SEQ ID NO: 16 - protein, 249 aa, Artificial Sequence
  scFv of humanized 12H10.G7 GB96(VL-VH)
DIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITRDTSASTAYMELSSLRSEDTAVYHCARWRQLGSLDSWGQGTTVTVSS

SEQ ID NO: 17 - protein, 118 aa, Artificial Sequence
  Humanized 12H10.G7 GB89/GB97-VH
QVQLVQSGAEVKKPGASVKVSCKASGYTFTRYVMHWVRQAPGQRLEWMGFINPYNDDTKYNEKFKGRVTITSDTSASTAYMELSSLRSEDTAVYYCARWRQLGSLDSWGQGTTVTVSS

SEQ ID NO: 18 - protein, 517 aa, Artificial Sequence
  extracellular region of hFLT3-ITD
NQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNIS

SEQ ID NO: 19 - protein, 249 aa, Artificial Sequence
  scFv of humanized 12H10.G7 GB89(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYYCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIK

SEQ ID NO: 20 - protein, 249 aa, Artificial Sequence
  scFv of humanized 12H10.G7 GB97(VL-VH)
DIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYYCARWRQLGSLDSWGQGTTVTVSS

SEQ ID NO: 21 - protein, 226 aa, Homo sapiens
  wild-type human IgG1 Fc sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 111 aa, Artificial Sequence
  Humanized 12H10.G7 GB90/GB98-VL
DIVMTQSPDSLAVSLGERATINCRASESVDTYGSSFVHWYQQKPGQPPKLLIYLASNLESGVPDRFSGSGSRTDFTLTISSLQAEDAATYYCQQNNEEPWTFGGGTKVEIK

SEQ ID NO: 23 - protein, 249 aa, Artificial Sequence
  scFv of humanized 12H10.G7 GB90(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIK

SEQ ID NO: 24 - protein, 249 aa, Artificial Sequence
  scFv of humanized 12H10.G7 GB98(VL-VH)
DIVMTQSPDSLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSS

SEQ ID NO: 25 - protein, 517 aa, Artificial Sequence
  extracellular region of hFLT3-T227M
NQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGMDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNIS

SEQ ID NO: 26 - protein, 111 aa, Artificial Sequence
  scFv of humanized 12H10.G7-VL
DIVMTQSPASLAVSLGERATINCRASESVDTYGSSFVHWYQQKPGQPPKLLIYLASNLESGVPDRFSGSGSGTDFTLTISSLQAEDAATYYCQQNNEEPWTFGGGTKVEIK

SEQ ID NO: 27 - protein, 249 aa, Artificial Sequence
  scFv of humanized 12H10.G7 GB91(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSGTDFTLTISSLQAEDAATYYCQQNNEEPWTFGCGTKVEIK

SEQ ID NO: 28 - protein, 249 aa, Artificial Sequence
  scFv of humanized 12H10.G7 GB99(VL-VH)
DIVMTQSPASLAVSLGERATINCRASESVDTYGSSFVHWYQQKPGQPPKLLIYLASNLESGVPDRFSGSGSGTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSS

SEQ ID NO: 29 - protein, 118 aa, Artificial Sequence
  Humanized 14A5.E8 consensus-VH
  misc_feature (30)..(30): Xaa = Phe or Thr
  misc_feature (31)..(31): Xaa = Ser or Tyr
  misc_feature (40)..(40): Xaa = Ala or Arg
  misc_feature (44)..(44): Xaa = Cys or Gly
  misc_feature (72)..(72): Xaa = Val or Glu
  misc_feature (76)..(76): Xaa = Ser or Tyr
  misc_feature (100)..(100): Xaa = Asn or Val
  misc_feature (104)..(104): Xaa = Thr or Tyr
QVQLVQSGAEVKKPGASVKVSCKVSGYTFXXYWINWVRQXPGKXLEWMGNIYPGSSIINYNENFKNRVTMTXDTSXDTAYMELSSLRSEDTAVYYCARRXVYLXFDYWGQGTLVTVSS

SEQ ID NO: 30 - protein, 111 aa, Artificial Sequence
  Humanized 12H10.G7 GB92/GB100-VL
DIVMTQSPASLAVSLGERATINCRASESVDTYGSSFVHWYQQKPGQPPKLLIYLASNLESGVPDRFSGSGSRTDFTLTISSLQAEDVATYYCQQNNEEPWTFGGGTKVEIK

SEQ ID NO: 31 - protein, 249 aa, Artificial Sequence
  scFv of humanized 12H10.G7 GB92(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDVATYYCQQNNEEPWTFGCGTKVEIK

SEQ ID NO: 32 - protein, 247 aa, Artificial Sequence
  scFv of humanized 12H10.G7 GB100(VL-VH)
DIVMTQSPASLAVSLGERATINCRASESVDTYGSSFVHWYQQKPGQPPKLLIYLASNLESGVPDRFSGSGSRTDFTLTISSLQAEDVATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTV

SEQ ID NO: 33 - protein, 6 aa, Artificial Sequence
  4H2.E3-VH CDR2
NPYSDG

SEQ ID NO: 34 - protein, 111 aa, Artificial Sequence
  Humanized 12H10.G7 GB93/GB101-VL
DIVMTQSPASLAVSLGERATINCRASESVDTYGSSFVHWYQQKPGQPPKLLIYLASNLESGVPDRFSGSGSRTDFTLTISSLQAEDAAVYYCQQNNEEPWTFGGGTKVEIK

SEQ ID NO: 35 - protein, 249 aa, Artificial Sequence
  scFv of humanized 12H10.G7 GB93(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDAAVYYCQQNNEEPWTFGCGTKVEIK

SEQ ID NO: 36 - protein, 249 aa, Artificial Sequence
  scFv of humanized 12H10.G7 GB101(VL-VH)
DIVMTQSPASLAVSLGERATINCRASESVDTYGSSFVHWYQQKPGQPPKLLIYLASNLESGVPDRFSGSGSRTDFTLTISSLQAEDAAVYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSS

SEQ ID NO: 37 - protein, 118 aa, Artificial Sequence
  Humanized 12H10.G7 GB94/GB102-VH
QVQLVQSGAEVKKPGASVKVSCKASGYTFTRYVMHWVRQAPGQRLEWMGFINPYNDDTKYNEKFKGRVTITRDTSASTAYMELSSLRSEDTAVYYCARWRQLGSLDSWGQGTTVTVSS

SEQ ID NO: 38 - protein, 111 aa, Artificial Sequence
  Humanized 12H10.G7 GB94/GB102-VL
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGGGTKVEIK

SEQ ID NO: 39 - protein, 249 aa, Artificial Sequence
  scFv of humanized 12H10.G7 GB94(VH-VL)
QVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLDSWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 40 - protein, 249 aa, Artificial Sequence
  scFv of humanized 12H10.G7 GB102(VL-VH)
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLDSWGQGTTVTVSS

SEQ ID NO: 41 - protein, 118 aa, Artificial Sequence
  Humanized 12H10.G7 GB102 D101E-VH
QVQLVQSGAEVKKPGASVKVSCKASGYTFTRYVMHWVRQAPGQRLEWMGFINPYNDDTKYNEKFKGRVTITRDTSASTAYMELSSLRSEDTAVYYCARWRQLGSLESWGQGTTVTVSS

SEQ ID NO: 42 - protein, 111 aa, Artificial Sequence
  Humanized 12H10.G7 GB102 -VL
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 43 - protein, 249 aa, Artificial Sequence
  scFv of humanized 12H10.G7 GB102 D101E (VH-VL)
QVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLESWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 44 - protein, 249 aa, Artificial Sequence
  scFv of humanized 12H10.G7 GB102 D101E (VL-VH)
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLESWGQGTTVTVSS

SEQ ID NO: 45 - protein, 118 aa, Artificial Sequence
  Humanized 12H10.G7 GB102 M34I-VH
QVQLVQSGAEVKKPGASVKVSCKASGYTFTRYVIHWVRQAPGQRLEWMGFINPYNDDTKYNEKFKGRVTITRDTSASTAYMELSSLRSEDTAVYYCARWRQLGSLDSWGQGTTVTVSS

SEQ ID NO: 46 - protein, 9 aa, Artificial Sequence
  14H8.E7-VL CDR3
QQWSSKSPT

SEQ ID NO: 47 - protein, 249 aa, Artificial Sequence
  scFv of humanized 12H10.G7 GB102 M34I (VH-VL)
QVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLDSWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 48 - protein, 249 aa, Artificial Sequence
  scFv of humanized 12H10.G7 GB102 M34I (VL-VH)
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLDSWGQGTTVTVSS

SEQ ID NO: 49 - protein, 118 aa, Artificial Sequence
  Humanized 12H10.G7 GB102 M34I/D101E-VH
QVQLVQSGAEVKKPGASVKVSCKASGYTFTRYVIHWVRQAPGQRLEWMGFINPYNDDTKYNEKFKGRVTITRDTSASTAYMELSSLRSEDTAVYYCARWRQLGSLESWGQGTTVTVSS

SEQ ID NO: 50 - protein, 9 aa, Artificial Sequence
  Humanized 12H10.G7 GB102 M34I/D101E -CDR3
WRQLGSLES

SEQ ID NO: 51 - protein, 249 aa, Artificial Sequence
  scFv of humanized 12H10.G7 GB102 M34I/D101E (VH-VL)
QVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLESWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 52 - protein, 249 aa, Artificial Sequence
  scFv of humanized 12H10.G7 GB102 M34I/D101E (VL-VH)
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLESWGQGTTVTVSS

SEQ ID NO: 53 - protein, 118 aa, Artificial Sequence
  Humanized 12H10.G7 consensus 1-VH
  misc_feature (34)..(34): Xaa = Met or Ile
  misc_feature (106)..(106): Xaa = Glu or Asp
QVQLVQSGAEVKKPGASVKVSCKASGYTFTRYVXHWVRQAPGQRLEWMGFINPYNDDTKYNEKFKGRVTITRDTSASTAYMELSSLRSEDTAVYYCARWRQLGSLXSWGQGTTVTVSS

SEQ ID NO: 54 - protein, 9 aa, Artificial Sequence
  Humanized 14A5.E8 consensus VH CDR3
  misc_feature (2)..(2): Xaa = Asn or Val
  misc_feature (6)..(6): Xaa = Thr or Tyr
RXVYLXFDY

SEQ ID NO: 55 - protein, 9 aa, Artificial Sequence
  Humanized 12H10.G7 consensus 1-VH CDR3
  misc_feature (8)..(8): Xaa = Glu or Asp
WRQLGSLXS

SEQ ID NO: 56 - protein, 118 aa, Artificial Sequence
  Humanized 12H10.G7 consensus 2 -VH
  misc_feature (72)..(72): Xaa = Ser or Arg
  misc_feature (95)..(95): Xaa = Tyr or His
QVQLVQSGAEVKKPGASVKVSCKASGYTFTRYVMHWVRQAPGQRLEWMGFINPYNDDTKYNEKFKGRVTITXDTSASTAYMELSSLRSEDTAVYXCARWRQLGSLDSWGQGTTVTVSS

SEQ ID NO: 57 - protein, 111 aa, Artificial Sequence
  Humanized 12H10.G7 consensus 2 -VL
  misc_feature (9)..(9): Xaa = Ala or Asp
  misc_feature (72)..(72): Xaa = Arg or Gly
  misc_feature (87)..(87): Xaa = Ala or Val
  misc_feature (89)..(89): Xaa = Thr or Val
DIVMTQSPXSLAVSLGERATINCRASESVDTYGSSFVHWYQQKPGQPPKLLIYLASNLESGVPDRFSGSGSXTDFTLTISSLQAEDXAXYYCQQNNEEPWTFGGGTKVEIK

SEQ ID NO: 58 - protein, 118 aa, Artificial Sequence
  Humanized 12H10.G7 consensus 3-VH
  misc_feature (34)..(34): Xaa = Met or Ile
  misc_feature (106)..(106): Xaa = Glu or Asp
QVQLVQSGAEVKKPGASVKVSCKASGYTFTRYVXHWVRQAPGQCLEWMGFINPYNDDTKYNEKFKGRVTITRDTSASTAYMELSSLRSEDTAVYYCARWRQLGSLXSWGQGTTVTVSS

SEQ ID NO: 59 - protein, 7 aa, Artificial Sequence
  Humanized 14A5.E8 consensus VH CDR1
  misc_feature (5)..(5): Xaa = Pro or Thr
  misc_feature (6)..(6): Xaa = Ser or Tyr
GYTFXXY

SEQ ID NO: 60 - protein, 118 aa, Artificial Sequence
  14A5.E8-VH
EVQLQESGAELVQPGASVRLSCKASGYTFTSYWINWVKQRPGQGLEWIGNIYPGSSIINYNENFKNRATLTVDTSSSTAYMQLSSLTSDDSAVYYCARRVVYLYFDYWGQGTTLTVSS

SEQ ID NO: 61 - protein, 106 aa, Artificial Sequence
  14A5.E8-VL
QIVLTQSPAIMSASPGEKVTMTCSASSSVSYMHWYQQKSGTSPKRWIYDTSKLASGVPARFSGSGSGTSYSLTISSMEAEDAATYYCQQWTSKSPTFGGGTKLEIK

SEQ ID NO: 62 - protein, 7 aa, Artificial Sequence
  14A5.E8-VH CDR1
GYTFTSY

SEQ ID NO: 63 - protein, 6 aa, Artificial Sequence
  14A5.E8-VH or Humanized 14A5.E8 consensus VH CDR2
YPGSSI

SEQ ID NO: 64 - protein, 9 aa, Artificial Sequence
  14A5.E8-VH CDR3
RVVYLYFDY

SEQ ID NO: 65 - protein, 10 aa, Artificial Sequence
  14A5.E8-VL CDR1
SASSSVSYMH

SEQ ID NO: 66 - protein, 7 aa, Artificial Sequence
  14A5.E8-VL or Humanized 14A5.E8 consensus VL CDR2
DTSKLAS

SEQ ID NO: 67 - protein, 9 aa, Artificial Sequence
  14A5.E8-VL or Humanized 14A5.E8 consensus VL CDR3
QQWTSKSPT

SEQ ID NO: 68 - protein, 118 aa, Artificial Sequence
  Humanized 14A5.E8 1551/1552-VH
QVQLVQSGAEVKKPGASVKVSCKVSGYTFTSYWINWVRQRPGKGLEWMGNIYPGSSIINYNENFKNRVTMTVDTSSDTAYMELSSLRSEDTAVYYCARRVVYLYFDYWGQGTLVTVSS

SEQ ID NO: 69 - protein, 106 aa, Artificial Sequence
  Humanized 14A5.E8 1551/1552-VL
EIVLTQSPATLSLSPGEKATLSCSASSSVSYMHWYQQKPGQAPRLLIYDTSKLASGIPARFSGSGSGTSFTLTISSLEPEDAAVYYCQQWTSKSPTFGGGTKVEIK

SEQ ID NO: 70 - protein, 244 aa, Artificial Sequence
  scFv of humanized 14A5.E8 1551(VH-VL)
QVQLVQSGAEVKKPGASVKVSCKVSGYTFTSYWINWVRQRPGKCLEWMGNIYPGSSIINYNENFKNRVTMTVDTSSDTAYMELSSLRSEDTAVYYCARRVVYLYFDYWGQGTLVTVSSGGGGSGGGGSGGGGSGGGGSEIVLTQSPATLSLSPGEKATLSCSASSSVSYMHWYQQKPGQAPRLLIYDTSKLASGIPARFSGSGSGTSFTLTISSLEPEDAAVYYCQQWTSKSPTFGCGTKVEIK

SEQ ID NO: 71 - protein, 244 aa, Artificial Sequence
  scFv of humanized 14A5.E8 1552(VL-VH)
EIVLTQSPATLSLSPGEKATLSCSASSSVSYMHWYQQKPGQAPRLLIYDTSKLASGIPARFSGSGSGTSFTLTISSLEPEDAAVYYCQQWTSKSPTFGCGTKVEIKGGGGSGGGGSGGGGSGGGGSQVQLVQSGAEVKKPGASVKVSCKVSGYTFTSYWINWVRQRPGKCLEWMGNIYPGSSIINYNENFKNRVTMTVDTSSDTAYMELSSLRSEDTAVYYCARRVVYLYFDYWGQGTLVTVSS

SEQ ID NO: 72 - protein, 118 aa, Artificial Sequence
  Humanized 14A5.E8 1553/1554-VH
QVQLVQSGAEVKKPGASVKVSCKVSGYTFTSYWINWVRQAPGKGLEWMGNIYPGSSIINYNENFKNRVTMTEDTSTDTAYMELSSLRSEDTAVYYCARRVVYLYFDYWGQGTLVTVSS

SEQ ID NO: 73 - protein, 106 aa, Artificial Sequence
  Humanized 14A5.E8 1553/1554-VL
EIVLTQSPATLSLSPGERATLSCSASSSVSYMHWYQQKPGQAPRLLIYDTSKLASGIPARFSGSGSGTDFTLTISSLEPEDFAVYYCQQWTSKSPTFGGGTKVEIK

SEQ ID NO: 74 - protein, 244 aa, Artificial Sequence
  scFv of humanized 14A5.E8 1553(VH-VL)
QVQLVQSGAEVKKPGASVKVSCKVSGYTFTSYWINWVRQAPGKCLEWMGNIYPGSSIINYNENFKNRVTMTEDTSTDTAYMELSSLRSEDTAVYYCARRVVYLYFDYWGQGTLVTVSSGGGGSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCQQWTSKSPTFGCGTKVEIK

SEQ ID NO: 75 - protein, 244 aa, Artificial Sequence
  scFv of humanized 14A5.E8 1554(VL-VH)
EIVLTQSPATLSLSPGERATLSCSASSSVSYMHWYQQKPGQAPRLLIYDTSKLASGIPARFSGSGSGTDFTLTISSLEPEDFAVYYCQQWTSKSPTFGCGTKVEIKGGGGSGGGGSGGGGSGGGGSQVQLVQSGAEVKKPGASVKVSCKVSGYTFTSYWINWVRQAPGKCLEWMGNIYPGSSIINYNENFKNRVTMTEDTSTDTAYMELSSLRSEDTAVYYCARRVVYLYFDYWGQGTLVTVSS

SEQ ID NO: 76 - protein, 118 aa, Artificial Sequence
  Humanized 14A5.E8 1689-VH
QVQLVQSGAEVKKPGASVKVSCKVSGYTFPYYWINWVRQAPGKGLEWMGNIYPGSSIINYNENFKNRVTMTEDTSTDTAYMELSSLRSEDTAVYYCARRNVYLTFDYWGQGTLVTVSS

SEQ ID NO: 77 - protein, 106 aa, Artificial Sequence
  Humanized 14A5.E8 1689-VL
EIVLTQSPATLSLSPGERATLSCSASSSVSYIHWYQQKPGQAPRLLIYDTSKLASGIPARFSGSGSGTDFTLTISSLEPEDFAVYYCQQWTSKSPTFGGGTKVEIK

SEQ ID NO: 78 - protein, 7 aa, Artificial Sequence
  Humanized 14A5.E8 1689-VH CDR1
GYTFPYY

SEQ ID NO: 79 - protein, 9 aa, Artificial Sequence
  Humanized 14A5.E8 1689-VH CDR3
RNVYLTFDY

SEQ ID NO: 80 - protein, 10 aa, Artificial Sequence
  Humanized 14A5.E8 1689-VL CDR1
SASSSVSYIH

SEQ ID NO: 81 - protein, 244 aa, Artificial Sequence
  scFv of humanized 14A5.E8 1689 (VH-VL)
QVQLVQSGAEVKKPGASVKVSCKVSGYTFPYYWINWVRQAPGKCLEWMGNIYPGSSIINYNENFKNRVTMTEDTSTDTAYMELSSLRSEDTAVYYCARRNVYLTFDYWGQGTLVTVSSGGGGSGGGGSGGGGSGGGGSEIVLTQSPATLSLSPGERATLSCSASSSVSYIHWYQQKPGQAPRLLIYDTSKLASGIPARFSGSGSGTDFTLTISSLEPEDFAVYYCQQWTSKSPTFGCGTKVEIK

SEQ ID NO: 82 - protein, 244 aa, Artificial Sequence
  scFv of humanized 14A5.E8 1689 (VL-VH)
EIVLTQSPATLSLSPGERATLSCSASSSVSYIHWYQQKPGQAPRLLIYDTSKLASGIPARFSGSGSGTDFTLTISSLEPEDFAVYYCQQWTSKSPTFGCGTKVEIKGGGGSGGGGSGGGGSGGGGSQVQLVQSGAEVKKPGASVKVSCKVSGYTFPYYWINWVRQAPGKCLEWMGNIYPGSSIINYNENFKNRVTMTEDTSTDTAYMELSSLRSEDTAVYYCARRNVYLTFDYWGQGTLVTVSS

SEQ ID NO: 83 - protein, 106 aa, Artificial Sequence
  14H8.E7-VL
QIVLTQSPAIMSASPGEKVTMTCSASSSVSYMHWYQQKSGTSPKRWIFDTSKLASGVPVRFSGSGSGTSYSLTITNMETEDAATYYCQQWSSKSPTFGGGTKLEIK

SEQ ID NO: 84 - protein, 106 aa, Artificial Sequence
  Humanized 14A5.E8 consensus VL
  misc_feature (32)..(32): Xaa = Met or Ile
EIVLTQSPATLSLSPGERATLSCSASSSVSYXHWYQQKPGQAPRLLIYDTSKLASGIPARFSGSGSGTDFTLTISSLEPEDFAVYYCQQWTSKSPTFGGGTKVEIK

SEQ ID NO: 85 - protein, 118 aa, Artificial Sequence
  11F4.B9-VH
EVQLQESGPELVKPGASVKISCKASGYSFTGYYIHWVKQGPEKSLEWIGEIIPSTGSTIYNQKFKAKATLTVDKSSSTAYLQLKSLTSEDSAVYYCERWGDYYGRDYWGQGTSVTVSS

SEQ ID NO: 86 - protein, 10 aa, Artificial Sequence
  Humanized 14A5.E8 consensus VL CDR1
  misc_feature (9)..(9): Xaa = Met or Ile
SASSSVSYXH

SEQ ID NO: 87 - protein, 7 aa, Artificial Sequence
  11F4.B9-VH CDR1
GYSFTGY

SEQ ID NO: 88 - protein, 6 aa, Artificial Sequence
  11F4.B9-VH CDR2
IPSTGS

SEQ ID NO: 89 - protein, 9 aa, Artificial Sequence
  11F4.B9-VH CDR3
WGDYYGRDY

SEQ ID NO: 90 - protein, 111 aa, Artificial Sequence
  11F4.B9-VL
DIVLTQSPASLAVSLGQRATISCRASESVDIYGNSFMHWYQQKPGQPPKLLIYRASNLESGIPARFSGSGSRTDFTLTINPVEADDVATYYCQQSNEDPRTFGGGTKLEIK

SEQ ID NO: 91 - protein, 15 aa, Artificial Sequence
  11F4.B9-VL CDR1
RASESVDIYGNSFMH

SEQ ID NO: 92 - protein, 7 aa, Artificial Sequence
  11F4.B9-VL CDR2
RASNLES

SEQ ID NO: 93 - protein, 9 aa, Artificial Sequence
  11F4.B9-VL CDR3
QQSNEDPRT

SEQ ID NO: 94 - protein, 111 aa, Artificial Sequence
  Humanized 11F4.B9-VL
DIVMTQSPASLAVSLGERATINCRASESVDIYGNSFMHWYQQKPGQPPKLLIYRASNLESGVPDRFSGSGSRTDFTLTINSLQAEDVATYYCQQSNEDPRTFGGGTKVEIK

SEQ ID NO: 95 - protein, 120 aa, Artificial Sequence
  4A4.A3-VH
QVTLKESGPGILQPSQTLSLTCSFSGFSLTTYGMGVGWIRQPSGKGLEWLANIWFNDNKYYNSTLKSRLTISKDTSNNQVFLKISSVDTTDTATYYCAQITTVVGTFDYWGQGSPLTVSP

SEQ ID NO: 96 - protein, 108 aa, Artificial Sequence
  4A4.A3-VL
RIVMTQSPTTMAASPGEKITITCSASSSISSIYLHWYQQKPGFSPKLLIFRTSDLASGVPPRFGGSGSGTSYSLTIGTMEAEDVATYYCQQGSSFPRTFGGGTKLEIK

SEQ ID NO: 97 - protein, 9 aa, Artificial Sequence
  4A4.A3-VH CDR1
GFSLTTYGM

SEQ ID NO: 98 - protein, 6 aa, Artificial Sequence
  4A4.H7-VH CDR2
YPNTGI

SEQ ID NO: 99 - protein, 5 aa, Artificial Sequence
  4A4.A3-VH CDR2
WFNDN

SEQ ID NO: 100 - protein, 10 aa, Artificial Sequence
  4A4.A3-VH CDR3
ITTVVGTFDY

SEQ ID NO: 101 - protein, 12 aa, Artificial Sequence
  4A4.A3-VL CDR1
SASSSISSIYLH

SEQ ID NO: 102 - protein, 7 aa, Artificial Sequence
  4A4.A3-VL CDR2
RTSDLAS

SEQ ID NO: 103 - protein, 9 aa, Artificial Sequence
  4A4.A3-VL CDR3
QQGSSFPRT

SEQ ID NO: 104 - protein, 118 aa, Artificial Sequence
  4A4.H7-VH
EVQLQESGPELVKPGASVKISCKASGYSFTGYYIHWVKQSPEESLEWIGEIYPNTGITTYNQKFTAKATLTVDKSSNTAYMQLKSLTSEDSAVYYCTRWGDYYGRDYWGQGTSVTVSS

SEQ ID NO: 105 - protein, 111 aa, Artificial Sequence
  4A4.H7-VL
DIVLTQSPASLAVSLGQRATISCRASETVDTHGNSFMHWYQQKPGQPPKLLIYRASNLESGIPARFSGSGSRTDFTLTINPVEADDVATYYCQQSNEDPRTFGGGTKLEIK

SEQ ID NO: 106 - protein, 15 aa, Artificial Sequence
  4A4.H7-VL CDR1
RASETVDTHGNSFMH

SEQ ID NO: 107 - protein, 118 aa, Artificial Sequence
  15A11.C8-VH
EVQLQESGGGLVKTGGSRKLSCAASGFTFSDYGMHWVRHTPEKGLEWVVYISSGGNTIFYTDTVKGRFTISRDNAKNTLFLQMTSLRSEDTAVYFCVRQGYYYAMDYWGQGASVTVSS

SEQ ID NO: 108 - protein, 107 aa, Artificial Sequence
  15A11.C8-VL
DIQMTQTTSSLSASLGDRVTIRCRASQDITNYLNWYQQKPDGAVKLLISYTSILQSGVPSRFSGSGSGTDYSLTISNLEQGDVATYFCQQGSSLPWTFGGGTKLEIK

SEQ ID NO: 109 - protein, 7 aa, Artificial Sequence
  15A11.C8-VH CDR1
GFTFSDY

SEQ ID NO: 110 - protein, 6 aa, Artificial Sequence
  15A11.C8-VH CDR2
SSGGNT

SEQ ID NO: 111 - protein, 9 aa, Artificial Sequence
  15A11.C8-VH CDR3
QGYYYAMDY

SEQ ID NO: 112 - protein, 11 aa, Artificial Sequence
  15A11.C8-VL CDR1
RASQDITNYLN

SEQ ID NO: 113 - protein, 7 aa, Artificial Sequence
  15A11.C8-VL CDR2
YTSILQS

SEQ ID NO: 114 - protein, 9 aa, Artificial Sequence
  15A11.C8-VL CDR3
QQGSSLPWT

SEQ ID NO: 115 - protein, 114 aa, Artificial Sequence
  12C9.E5-VH
EVQLQESGAELVRPGASVKLSCKASGYIFTDYEIHWVKQTPVHGLEWIGAIDPETGITAYSQKFKGKATLTTDTSSSTAYMEFRSLTSEDSAVYYCTRGGLLYWGQGTSVTVSS

SEQ ID NO: 116 - protein, 112 aa, Artificial Sequence
  12C9.E5-VL
DVVMTQTPLSLSVTIGQPASISCKSSQSLLYSDGETYLNWLQQRPGQSPKRLMYQVSKLDPGIPDRFSGSGSETDFTLKISRVEAEDLGIYYCLQGTFYPHTFGGGTKLEIK

SEQ ID NO: 117 - protein, 7 aa, Artificial Sequence
  12C9.E5-VH CDR1
GYIFTDY

SEQ ID NO: 118 - protein, 6 aa, Artificial Sequence
  12C9.E5-VH CDR2
DPETGI

SEQ ID NO: 119 - protein, 5 aa, Artificial Sequence
  12C9.E5-VH CDR3
GGLLY

SEQ ID NO: 120 - protein, 16 aa, Artificial Sequence
  12C9.E5-VL CDR1
KSSQSLLYSDGETYLN

SEQ ID NO: 121 - protein, 7 aa, Artificial Sequence
  12C9.E5-VL CDR2
QVSKLDP

SEQ ID NO: 122 - protein, 9 aa, Artificial Sequence
  12C9.E5-VL CDR3
LQGTFYPHT

SEQ ID NO: 123 - protein, 118 aa, Artificial Sequence; identical to SEQ ID NO: 104
  1A2.A3 VH

SEQ ID NO: 124 - protein, 111 aa, Artificial Sequence; identical to SEQ ID NO: 105
  1A2.A3-VL

SEQ ID NO: 125 - protein, 120 aa, Artificial Sequence
  4H2.E3-VH
EVQLQESGPELVKPGASVKMSCKASGYTFTSYLMHWMKQKPGQGLEWIGYINPYSDGIKYNEKFRDKATLTSDKSSNTAYMELSSLTSEDSAVYYCAHSSGYVGYAMDYWGQGTSVTVSS

SEQ ID NO: 126 - protein, 112 aa, Artificial Sequence
  4H2.E3-VL
GIVMTQTTPSVPVTPGESVSISCRSSKSLLHSNGNTYLYWFLQRPGQSPQLLIYRMSNLASGVPDRFSGSGSGTTFTLRISRVEAEDVGVYYCMQHLEYPFTFGSGTKLEIK

SEQ ID NO: 127 - protein, 11 aa, Artificial Sequence
  4H2.E3-VH CDR3
SSGYVGYAMDY

SEQ ID NO: 128 - protein, 16 aa, Artificial Sequence
  4H2.E3-VL CDR1
RSSKSLLHSNGNTYLY

SEQ ID NO: 129 - protein, 7 aa, Artificial Sequence
  4H2.E3-VL CDR2
RMSNLAS

SEQ ID NO: 130 - protein, 9 aa, Artificial Sequence
  4H2.E3-VL CDR3
MQHLEYPFT

SEQ ID NO: 131 - protein, 118 aa, Artificial Sequence
  14H8.E7-VH
EVQLQESGAELVKPGASVKLSCKASGYTFTNYWINWLKQRPGQGLEWIGNIYPGSTIINYNEKFKNKATLTVDTSSSTAYMQLSSLTSDDSAVYYCARRVVYLYFDSWGQGTTLTVSS

SEQ ID NO: 132 - protein, 7 aa, Artificial Sequence
  14H8.E7-VH CDR1
GYTFTNY

SEQ ID NO: 133 - protein, 6 aa, Artificial Sequence
  14H8.E7-VH CDR2
YPGSTI

SEQ ID NO: 134 - protein, 9 aa, Artificial Sequence
  14H8.E7-VH CDR3
RVVYLYFDS

SEQ ID NO: 135 - protein, 517 aa, Artificial Sequence; identical to SEQ ID NO: 18
  mature human FLT3 extracellular domain

SEQ ID NO: 136 - protein, 226 aa, Artificial Sequence; identical to SEQ ID NO: 21
  wild-type human IgG1 Fc sequence

SEQ ID NO: 137 - protein, 20 aa, Artificial Sequence
  ((G4S)4) linker
GGGGSGGGGSGGGGSGGGGS

SEQ ID NO: 138 - protein, 117 aa, Artificial Sequence
  ADI-27705-VH
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 139 - protein, 107 aa, Artificial Sequence
  ADI-27705-VL
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 140 - protein, 9 aa, Artificial Sequence
  ADI-27705-VH CDR1
GSFSGYYWS

SEQ ID NO: 141 - protein, 16 aa, Artificial Sequence
  ADI-27705-VH CDR2
EIDHSGSTNYNPSLKS

SEQ ID NO: 142 - protein, 11 aa, Artificial Sequence
  ADI-27705-VH CDR3
ARARGPWSFDP

SEQ ID NO: 143 - protein, 117 aa, Artificial Sequence; identical to SEQ ID NO: 138
  ADI-27724-VH

SEQ ID NO: 144 - protein, 108 aa, Artificial Sequence
  ADI-27724-VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 145 - protein, 117 aa, Artificial Sequence; identical to SEQ ID NO: 138
  ADI-27740(A40)-VH

SEQ ID NO: 146 - protein, 106 aa, Artificial Sequence
  ADI-27740(A40)-VL
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 147 - protein, 117 aa, Artificial Sequence; identical to SEQ ID NO: 138
  ADI-27741-VH

SEQ ID NO: 148 - protein, 106 aa, Artificial Sequence
  ADI-27741-VL
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 149 - protein, 117 aa, Artificial Sequence; identical to SEQ ID NO: 138
  ADI-27743-VH

SEQ ID NO: 150 - protein, 106 aa, Artificial Sequence
  ADI-27743-VL
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 151 - protein, 117 aa, Artificial Sequence
  ADI-28153-VH
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 152 - protein, 107 aa, Artificial Sequence
  ADI-28153-VL
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 153 - protein, 117 aa, Artificial Sequence; identical to SEQ ID NO: 138
  ADI-28226(C26)-VH

SEQ ID NO: 154 - protein, 107 aa, Artificial Sequence
  ADI-28226(C26)-VL
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 155 - protein, 117 aa, Artificial Sequence; identical to SEQ ID NO: 138
  ADI-28154-VH

SEQ ID NO: 156 - protein, 107 aa, Artificial Sequence
  ADI-28154-VL
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 157 - protein, 117 aa, Artificial Sequence; identical to SEQ ID NO: 138
  ADI-29399-VH

SEQ ID NO: 158 - protein, 106 aa, Artificial Sequence
  ADI-29399-VL
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 159 - protein, 117 aa, Artificial Sequence; identical to SEQ ID NO: 138
  ADI-29401-VH

SEQ ID NO: 160 - protein, 106 aa, Artificial Sequence
  ADI-29401-VL
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 161 - protein, 107 aa, Artificial Sequence
  ADI-29443(F43)-VL
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 162 - protein, 117 aa, Artificial Sequence; identical to SEQ ID NO: 138
  ADI-29403-VH

SEQ ID NO: 163 - protein, 106 aa, Artificial Sequence
  ADI-29403-VL
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 164 - protein, 117 aa, Artificial Sequence; identical to SEQ ID NO: 138
  ADI-29405-VH

SEQ ID NO: 165 - protein, 106 aa, Artificial Sequence
  ADI-29405-VL
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 166 - protein, 117 aa, Artificial Sequence; identical to SEQ ID NO: 138
  ADI-29407-VH

SEQ ID NO: 167 - protein, 106 aa, Artificial Sequence
  ADI-29407-VL
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 168 - protein, 117 aa, Artificial Sequence; identical to SEQ ID NO: 138
  ADI-29419-VH

SEQ ID NO: 169 - protein, 106 aa, Artificial Sequence
  ADI-29419-VL
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 170 - protein, 117 aa, Artificial Sequence; identical to SEQ ID NO: 138
  ADI-29421-VH

SEQ ID NO: 171 - protein, 106 aa, Artificial Sequence
  ADI-29421-VL
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 172 - protein, 117 aa, Artificial Sequence; identical to SEQ ID NO: 138
  ADI-29424-VH

SEQ ID NO: 173 - protein, 106 aa, Artificial Sequence
  ADI-29424-VL
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 174 - protein, 117 aa, Artificial Sequence; identical to SEQ ID NO: 138
  ADI-29425-VH

SEQ ID NO: 175 - protein, 106 aa, Artificial Sequence
  ADI-29425-VL
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 176 - protein, 117 aa, Artificial Sequence; identical to SEQ ID NO: 138
  ADI-29426-VH

SEQ ID NO: 177 - protein, 106 aa, Artificial Sequence
  ADI-29426-VL
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 178 - protein, 117 aa, Artificial Sequence; identical to SEQ ID NO: 138
  ADI-29429-VH

SEQ ID NO: 179 - protein, 107 aa, Artificial Sequence
  ADI-29429-VL
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 180 - protein, 117 aa, Artificial Sequence; identical to SEQ ID NO: 138
  ADI-29447(F47)-VH

SEQ ID NO: 181 - protein, 106 aa, Artificial Sequence
  ADI-29447(F47)-VL
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 182 - protein, 125 aa, Artificial Sequence
  ADI-27727-VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 183 - protein, 113 aa, Artificial Sequence
  ADI-27727-VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 184 - protein, 9 aa, Artificial Sequence
  ADI-27727-VH CDR1(non-Kabat)
GTFSSYAIS

SEQ ID NO: 185 - protein, 5 aa, Artificial Sequence
  ADI-27727-VH CDR1(non-Kabat)
SYAIS

SEQ ID NO: 186 - protein, 17 aa, Artificial Sequence
  ADI-27727-VH CDR2
GIIPIFGTANYAQKFQG

SEQ ID NO: 187 - protein, 17 aa, Artificial Sequence
  ADI-27727-VL CDR1
KSSQSVLYSSNNKNYLA

SEQ ID NO: 188 - protein, 7 aa, Artificial Sequence
  ADI-27727-VL CDR2
WASTRES

SEQ ID NO: 189 - protein, 18 aa, Artificial Sequence
  ADI-27727-VH CDR3 (non-Kabat)
ARGDSSIRHAYYYYGMDV

SEQ ID NO: 190 - protein, 16 aa, Artificial Sequence
  ADI-27727-VH CDR3 (non-Kabat)
GDSSIRHAYYYYGMDV

SEQ ID NO: 191 - protein, 9 aa, Artificial Sequence
  ADI-27727-VL CDR3
QQYYSTPIT

SEQ ID NO: 192 - protein, 121 aa, Artificial Sequence
  ADI-29443(F43)-VH
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 193 - protein, 11 aa, Artificial Sequence
  ADI-29443(F43)-VH CDR1(non-Kabat)
GSISSSSYYWG

SEQ ID NO: 194 - protein, 7 aa, Artificial Sequence
  ADI-29443(F43)-VH CDR1(non-Kabat)
SSSYYWG

SEQ ID NO: 195 - protein, 16 aa, Artificial Sequence
  ADI-29443(F43)-VH CDR2
SIYYSGSTYYNPSLKS

SEQ ID NO: 196 - protein, 13 aa, Artificial Sequence
  ADI-29443(F43)-VH CDR3 (non-Kabat)
ARGSDRFHPYFDY

SEQ ID NO: 197 - protein, 11 aa, Artificial Sequence
  ADI-29443(F43)-VH CDR3 (non-Kabat)
GSDRFHPYFDY

SEQ ID NO: 198 - protein, 11 aa, Artificial Sequence
  ADI-29443(F43)-VL CDR1
RASQSVSRYLA

SEQ ID NO: 199 - protein, 7 aa, Artificial Sequence
  ADI-29443(F43)-VL CDR2
DASNRAT

SEQ ID NO: 200 - protein, 9 aa, Artificial Sequence
  ADI-29443(F43)-VL CDR3
QQFDTWPPT

SEQ ID NO: 201 - protein, 117 aa, Artificial Sequence; identical to SEQ ID NO: 138
  ADI-29404(F04)-VH

SEQ ID NO: 202 - protein, 106 aa, Artificial Sequence
  ADI-29404(F04)-VL
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 203 - protein, 126 aa, Artificial Sequence
  ADI-28200-VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 204 - protein, 113 aa, Artificial Sequence [sequence omitted]

SEQ ID NO: 205 - protein, 17 aa, Artificial Sequence
  ADI-28200-VH CDR2
GIIPIFGTANYAQKFQG

SEQ ID NO: 206 - protein, 19 aa, Artificial Sequence
  ADI-28200-VH CDR3
ARRGRKASGSFYYYYGMDV

SEQ ID NO: 207 - protein, 17 aa, Artificial Sequence
  ADI-28200-VH CDR1
ESSQSLLNSGNQKNYLT

SEQ ID NO: 208 - protein, 9 aa, Artificial Sequence
  ADI-28200-VH CDR3
QNDYSYPYT

SEQ ID NO: 209 - protein, 126 aa, Artificial Sequence
  ADI-29379(E79)-VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 210 - protein, 107 aa, Artificial Sequence
  ADI-29379(E79)-VL
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 211 - protein, 9 aa, Artificial Sequence
  ADI-29379(E79)-VH CDR1(non-Kabat)
YTFTSYYMH

SEQ ID NO: 212 - protein, 5 aa, Artificial Sequence
  ADI-29379(E79)-VH CDR1(non-Kabat)
SYYMH

SEQ ID NO: 213 - protein, 17 aa, Artificial Sequence
  ADI-29379(E79)-VH CDR2
IINPSGGSTSYAQKFQG

SEQ ID NO: 214 - protein, 19 aa, Artificial Sequence
  ADI-29379(E79)-VH CDR3(non-Kabat)
ARGAPNYGDTTHDYYYMDV

SEQ ID NO: 215 - protein, 17 aa, Artificial Sequence
  ADI-29379(E79)-VH CDR3(non-Kabat)
GAPNYGDTTHDYYYMDV

SEQ ID NO: 216 - protein, 11 aa, Artificial Sequence
  ADI-29379(E79)-VH CDR1
RASQSVSSNLA

SEQ ID NO: 217 - protein, 7 aa, Artificial Sequence
  ADI-29379(E79)-VH CDR2
GASTRAT

SEQ ID NO: 218 - protein, 9 aa, Artificial Sequence
  ADI-29379(E79)-VH CDR3
QQYDDWPFT

SEQ ID NO: 219 - protein, 124 aa, Artificial Sequence
  ADI-29463(F63)-VH
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 220 - protein, 107 aa, Artificial Sequence
  ADI-29463(F63)-VL
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 221 - protein, 9 aa, Artificial Sequence
  ADI-29463(F63)-VH CDR1(non-Kabat)
YTFTGYYMH

SEQ ID NO: 222 - protein, 5 aa, Artificial Sequence
  ADI-29463(F63)-VH CDR1(non-Kabat)
GYYMH

SEQ ID NO: 223 - protein, 17 aa, Artificial Sequence
  ADI-29463(F63)-VH CDR2
WINPNSGGTNYAQKFQG

SEQ ID NO: 224 - protein, 17 aa, Artificial Sequence
  ADI-29463(F63)-VH CDR3(non-Kabat)
ARDTGEYYDTDDHGMDV

SEQ ID NO: 225 - protein, 15 aa, Artificial Sequence
  ADI-29463(F63)-VH CDR3(non-Kabat)
DTGEYYDTDDHGMDV

SEQ ID NO: 226 - protein, 11 aa, Artificial Sequence
  ADI-29463(F63)-VL CDR1
RASQSVSSNLA

SEQ ID NO: 227 - protein, 9 aa, Artificial Sequence
  ADI-29463(F63)-VL CDR3
QQDDYWPPT

SEQ ID NO: 228 - protein, 121 aa, Artificial Sequence
  ADI-27744(A44)-VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 229 - protein, 107 aa, Artificial Sequence
  ADI-27744(A44)-VL
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 230 - protein, 9 aa, Artificial Sequence
  ADI-27744(A44)-VH CDR1(non-Kabat)
FTFSSYAMS

SEQ ID NO: 231 - protein, 5 aa, Artificial Sequence
  ADI-27744(A44)-VH CDR1(non-Kabat)
SYAMS

SEQ ID NO: 232 - protein, 17 aa, Artificial Sequence
  ADI-27744(A44)-VH CDR2
AISGSGGSTYYADSVKG

SEQ ID NO: 233 - protein, 14 aa, Artificial Sequence
  ADI-27744(A44)-VH CDR3(non-Kabat)
AKDGGYYDSGAGDY

SEQ ID NO: 234 - protein, 12 aa, Artificial Sequence
  ADI-27744(A44)-VH CDR3(non-Kabat)
DGGYYDSGAGDY

SEQ ID NO: 235 - protein, 11 aa, Artificial Sequence
  ADI-27744(A44)-VL CDR1
RASQGIDSWLA

SEQ ID NO: 236 - protein, 7 aa, Artificial Sequence
  ADI-27744(A44)-VL CDR2
AASSLQS

SEQ ID NO: 237 - protein, 9 aa, Artificial Sequence
  ADI-27744(A44)-VL CDR3
QQGVSYPRT

SEQ ID NO: 238 - protein, 122 aa, Artificial Sequence
  ADI-27749(A49)-VH
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 239 - protein, 107 aa, Artificial Sequence
  ADI-27749(A49)-VL
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 240 - protein, 9 aa, Artificial Sequence
  ADI-27749(A49)-VH CDR1(non-Kabat)
FTFSSYSMN

SEQ ID NO: 241 - protein, 5 aa, Artificial Sequence
  ADI-27749(A49)-VH CDR1(non-Kabat)
SYSMN

SEQ ID NO: 242 - protein, 17 aa, Artificial Sequence
  ADI-27749(A49)-VH CDR2
SISSSSSYIYYADSVKG

SEQ ID NO: 243 - protein, 15 aa, Artificial Sequence
  ADI-27749(A49)-VH CDR3(non-Kabat)
ARGAPMGAAAGWFDP

SEQ ID NO: 244 - protein, 13 aa, Artificial Sequence
  ADI-27749(A49)-VH CDR3(non-Kabat)
GAPMGAAAGWFDP

SEQ ID NO: 245 - protein, 9 aa, Artificial Sequence
  ADI-27749(A49)-VL CDR3
QQGVSFPRT

SEQ ID NO: 246 - protein, 249 aa, Artificial Sequence
  scFv (VL-VH) with Q44C in VH and G100C in VL of ADI-27749(A49)
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPMGAAAGWFDPWGQGTLVTVSS

SEQ ID NO: 247 - protein, 125 aa, Artificial Sequence
  ADI-29378(E78)-VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 248 - protein, 107 aa, Artificial Sequence
  ADI-29378(E78)-VL
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 249 - protein, 17 aa, Artificial Sequence
  ADI-29378(E78)-VH CDR2
IINPSGGSTSYAQKFQG

SEQ ID NO: 250 - protein, 18 aa, Artificial Sequence
  ADI-29378(E78)-VH CDR3(non-Kabat)
AREGAGFAYGMDYYYMDV

SEQ ID NO: 251 - protein, 16 aa, Artificial Sequence
  ADI-29378(E78)-VH CDR3(non-Kabat)
EGAGFAYGMDYYYMDV

SEQ ID NO: 252 - protein, 11 aa, Artificial Sequence
  ADI-29378(E78)-VL CDR1
RASQSVSSYLA

SEQ ID NO: 253 - protein, 9 aa, Artificial Sequence
  ADI-29378(E78)-VL CDR3
QQSDNWPFT

SEQ ID NO: 254 - protein, 122 aa, Artificial Sequence
  A49MI-VH
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 255 - protein, 15 aa, Artificial Sequence
  A49MI-VH CDR3(non-Kabat)
ARGAPIGAAAGWFDP

SEQ ID NO: 256 - protein, 13 aa, Artificial Sequence
  A49MI-VH CDR3(non-Kabat)
GAPIGAAAGWFDP

SEQ ID NO: 257 - protein, 122 aa, Artificial Sequence
  A49MQ-VH
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 258 - protein, 15 aa, Artificial Sequence
  A49MQ-VH CDR3 (non-Kabat)
ARGAPQGAAAGWFDP

SEQ ID NO: 259 - protein, 12 aa, Artificial Sequence
  A49MQ-VH CDR3
GAPQGAAAGWFD

SEQ ID NO: 260 - protein, 122 aa, Artificial Sequence
  A49ML-VH
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 261 - protein, 15 aa, Artificial Sequence
  A49ML-VH CDR3 (non-Kabat)
ARGAPLGAAAGWFDP

SEQ ID NO: 262 - protein, 13 aa, Artificial Sequence
  A49ML-VH CDR3
GAPLGAAAGWFDP

SEQ ID NO: 263 - protein, 122 aa, Artificial Sequence
  A49MF-VH
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 264 - protein, 15 aa, Artificial Sequence
  A49MF-VH CDR3 (non-Kabat)
ARGAPFGAAAGWFDP

SEQ ID NO: 265 - protein, 13 aa, Artificial Sequence
  A49MF-VH CDR3
GAPFGAAAGWFDP

SEQ ID NO: 266 - protein, 122 aa, Artificial Sequence
  A49MV-VH
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 267 - protein, 15 aa, Artificial Sequence
  A49MV-VH CDR3 (non-Kabat)
ARGAPVGAAAGWFDP

SEQ ID NO: 268 - protein, 13 aa, Artificial Sequence
  A49MV-VH CDR3
GAPVGAAAGWFDP

SEQ ID NO: 269 - protein, 122 aa, Artificial Sequence
  A49-consensus-VH
  misc_feature (102)..(102): Xaa = Met, Leu, Ile, Val, Gln or Phe
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 270 - protein, 15 aa, Artificial Sequence
  A49-consensus-VH CDR3 (non-Kabat)
  misc_feature (6)..(6): Xaa = Met, Leu, Ile, Val, Gln or Phe
ARGAPXGAAAGWFDP

SEQ ID NO: 271 - protein, 13 aa, Artificial Sequence
  A49-consensus-VH CDR3
  misc_feature (4)..(4): Xaa = Met, Leu, Ile, Val, Gln or Phe
GAPXGAAAGWFDP

SEQ ID NO: 272 - protein, 121 aa, Artificial Sequence
  NKG2D binder in US9,273,136-VH
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 273 - protein, 110 aa, Artificial Sequence
  NKG2D binder in US9,273,136-VL
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 274 - protein, 115 aa, Artificial Sequence
  NKG2D binder in US7,879,985-VH
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 275 - protein, 108 aa, Artificial Sequence
  NKG2D binder in US7,879,985-VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 276 - protein, 11 aa, Artificial Sequence
  A49MI-VL CDR1
RASQGISSWLA

SEQ ID NO: 277 - protein, 477 aa, Artificial Sequence
  GB102-VL-VH-Fc
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLDSWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 278 - protein, 451 aa, Artificial Sequence
  A49MI-VH-CH1-Fc
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 279 - protein, 214 aa, Artificial Sequence
  A49MI-VL-CL
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 477 aa, Artificial Sequence
  A49-VL-VH-Fc
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPMGAAAGWFDPWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 281 - protein, 447 aa, Artificial Sequence
  GB102-VH-CH1-Fc
QVQLVQSGAEVKKPGASVKVSCKASGYTFTRYVMHWVRQAPGQGLEWMGFINPYNDDTKYNEKFKGRVTITRDTSASTAYMELSSLRSEDTAVYYCARWRQLGSLDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 282 - protein, 218 aa, Artificial Sequence
  GB102-VL-CL
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 472 aa, Artificial Sequence
  1553-VH-VL-Fc
QVQLVQSGAEVKKPGASVKVSCKVSGYTFTSYWINWVRQAPGKCLEWMGNIYPGSSIINYNENFKNRVTMTEDTSTDTAYMELSSLRSEDTAVYYCARRVVYLYFDYWGQGTLVTVSSGGGGSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCQQWTSKSPTFGCGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 284 - protein, 472 aa, Artificial Sequence
  1689-VH-VL-Fc
QVQLVQSGAEVKKPGASVKVSCKVSGYTFPYYWINWVRQAPGKCLEWMGNIYPGSSIINYNENFKNRVTMTEDTSTDTAYMELSSLRSEDTAVYYCARRNVYLTFDYWGQGTLVTVSSGGGGSGGGGSGGGGSGGGGSEIVLTQSPATLSLSPGERATLSCSASSSVSYIHWYQQKPGQAPRLLIYDTSKLASGIPARFSGSGSGTDFTLTISSLEPEDFAVYYCQQWTSKSPTFGCGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 285 - protein, 477 aa, Artificial Sequence
  GB102_M34I-VL-VH-Fc
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLDSWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 286 - protein, 477 aa, Artificial Sequence
  GB102_D101E-VL-VH-Fc
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLESWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 287 - protein, 477 aa, Artificial Sequence
  GB102_M34I_D101E-VL-VH-Fc
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLESWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 288 - protein, 477 aa, Artificial Sequence
  GB99-VL-VH-Fc
DIVMTQSPASLAVSLGERATINCRASESVDTYGSSFVHWYQQKPGQPPKLLIYLASNLESGVPDRFSGSGSGTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 289 - protein, 477 aa, Artificial Sequence
  GB89-VH-VL-Fc
QVQLVQSGAEVKKPGASVKVSCKASGYTFTRYVMHWVRQAPGQCLEWMGFINPYNDDTKYNEKFKGRVTITSDTSASTAYMELSSLRSEDTAVYYCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 290 - protein, 2 aa, Artificial Sequence
  Linker sequence
  REPEAT (1)..(2): GS can be repeated n times, n can be 1, 2, 3, 4, 5, 6, 7, 8, 9, 10, 11, 12, 13, 14, 15, 16, 17, 18, 19, or 20
GS

SEQ ID NO: 291 - protein, 3 aa, Artificial Sequence
  Linker sequence
  REPEAT (1)..(3): GGS can be repeated n times, n can be 1, 2, 3, 4, 5, 6, 7, 8, 9, 10, 11, 12, 13, 14, 15, 16, 17, 18, 19, or 20
GGS

SEQ ID NO: 292 - protein, 4 aa, Artificial Sequence
  Linker sequence
  REPEAT (1)..(4): GGGS can be repeated n times, n can be 1, 2, 3, 4, 5, 6, 7, 8, 9, 10, 11, 12, 13, 14, 15, 16, 17, 18, 19, or 20
GGGS

SEQ ID NO: 293 - protein, 4 aa, Artificial Sequence
  Linker sequence
  REPEAT (1)..(4): GGSG can be repeated n times, n can be 1, 2, 3, 4, 5, 6, 7, 8, 9, 10, 11, 12, 13, 14, 15, 16, 17, 18, 19, or 20
GGSG

SEQ ID NO: 294 - protein, 5 aa, Artificial Sequence
  Linker sequence
  REPEAT (1)..(5): GGSGG can be repeated n times, n can be 1, 2, 3, 4, 5, 6, 7, 8, 9, 10, 11, 12, 13, 14, 15, 16, 17, 18, 19, or 20
GGSGG

SEQ ID NO: 295 - protein, 5 aa, Artificial Sequence
  Linker sequence
  REPEAT (1)..(5): GGGGS can be repeated n times, n can be 1, 2, 3, 4, 5, 6, 7, 8, 9, 10, 11, 12, 13, 14, 15, 16, 17, 18, 19, or 20
GGGGS

SEQ ID NO: 296 - protein, 20 aa, Artificial Sequence
  Linker sequence
GSGSGSGSGSGSGSGSGSGS

SEQ ID NO: 297 - protein, 30 aa, Artificial Sequence
  Linker sequence
GGSGGSGGSGGSGGSGGSGGSGGSGGSGGS

SEQ ID NO: 298 - protein, 40 aa, Artificial Sequence
  Linker sequence
GGGSGGGSGGGSGGGSGGGSGGGSGGGSGGGSGGGSGGGS

SEQ ID NO: 299 - protein, 40 aa, Artificial Sequence
  Linker sequence
GGSGGGSGGGSGGGSGGGSGGGSGGGSGGGSGGGSGGGSG

SEQ ID NO: 300 - protein, 50 aa, Artificial Sequence
  Linker sequence
GGSGGGGSGGGGSGGGGSGGGGSGGGGSGGGGSGGGGSGGGGSGGGGSGG

SEQ ID NO: 301 - protein, 50 aa, Artificial Sequence
  Linker sequence
GGGGSGGGGSGGGGSGGGGSGGGGSGGGGSGGGGSGGGGSGGGGSGGGGS

SEQ ID NO: 302 - protein, 15 aa, Artificial Sequence
  Linker sequence
GGGGSGGGGSGGGGS

SEQ ID NO: 303 - protein, 100 aa, Artificial Sequence
  Linker sequence
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 304 - protein, 100 aa, Artificial Sequence
  Linker sequence
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 305 - protein, 106 aa, Artificial Sequence
  TriNKET variant with mutant light chain variable domain
DIQMTQSPSTLSASVGDRVTITCRASNSISSWLAWYQQKPGKAPKLLIYEASSTKSGVPSRFSGSGSGTEFTLTISSLQPDDFATYYCQQYDDLPTFGGGTKVEIK

SEQ ID NO: 306 - protein, 118 aa, Artificial Sequence
  Alpha-FLT3 mAb 4G8-Synimmune VH
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 307 - protein, 107 aa, Artificial Sequence
  Alpha-FLT3 mAb 4G8-Synimmune VL
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIK

SEQ ID NO: 308 - protein, 119 aa, Artificial Sequence
  Alpha-FLT3 mAb EB10-ImClone&Lilly VH
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSS

SEQ ID NO: 309 - protein, 112 aa, Artificial Sequence
  Alpha-FLT3 mAb EB10-ImClone&Lilly VL
DVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIK

SEQ ID NO: 310 - protein, 122 aa, Artificial Sequence
  Alpha-FLT3 mAb NC7 ImClone&Lilly VH
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 311 - protein, 107 aa, Artificial Sequence
  Alpha-FLT3 mAb NC7 ImClone&Lilly VL
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 312 - protein, 124 aa, Artificial Sequence
  Alpha-FLT3 mAb FL23(Amgen) VH
QVTLKESGPALVKPTETLTLTCTVSGFSFRNARMGVSWIRQPPGKALEWLAHIFSNDEKSYSTSLKSRLTISKDTSKSQVVLTLTNMDPVDTATYFCARMPEYSSGWSGAFDIWGQGTMVTVSS

SEQ ID NO: 313 - protein, 107 aa, Artificial Sequence
  Alpha-FLT3 mAb FL23(Amgen) VL
DIQMTQSPSSLSASVGDRVTITCRASQDIGYDLGWYQQKPGKAPKRLIYAASTLQSGVPSRFSGSGSGTEFTLIISSLQPEDFATYYCLQHNSFPWTFGQGTKVEIK

SEQ ID NO: 314 - protein, 124 aa, Artificial Sequence
  Alpha-FLT3 mAb FL39(Amgen) VH
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 315 - protein, 107 aa, Artificial Sequence
  Alpha-FLT3 mAb FL39(Amgen) VL
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 316 - protein, 129 aa, Artificial Sequence
  Alpha-FLT3 mAb FL61(Amgen) VH
QVQLVESGGGVVQPGRSLRLSCAASGFTFSSYGMHWVRQAPGKGLEWVAVISYDGSNEFYADSVKGRFTISRDNSKNTLYLQMNSLRAEDTAVYYCARGGEITMVRGVIGYYYYGMDVWGQGTTVTVSS

SEQ ID NO: 317 - protein, 107 aa, Artificial Sequence
  Alpha-FLT3 mAb FL61(Amgen) VL
DIQMTQSPSSLSASVGDRVTITCRASQSISSYLNWYQQKPGKAPKLLIYAASSLQSGVPSRFSGSGSGTEFTLTISSLQPEDFATYYCLQHNSYPLTFGGGTKVEIK

SEQ ID NO: 318 - protein, 517 aa, Artificial Sequence; identical to SEQ ID NO: 25
  extracellular region of hFLT3-T227M

SEQ ID NO: 319 - protein, 517 aa, Artificial Sequence; identical to SEQ ID NO: 18
  extracellular region of hFLT3-ITD

(19) United States
(12) Patent Application Publication
(10) Pub. No.: US 2024/0117054 A1
(43) Pub. Date: Apr. 11, 2024

(54) PROTEINS BINDING NKG2D, CD16 AND FLT3

(71) Applicant: Dragonfly Therapeutics, Inc., Waltham, MA (US)

(72) Inventors: Hemanta Baruah, Euless, TX (US); Gregory P. Chang, Medford, MA (US); Ann F. Cheung, Lincoln, MA (US); Asya Grinberg, Lexington, MA (US); Zong Sean Juo, Cambridge, MA (US); Thomas J. McQuade, Boston, MA (US); Daniel Fallon, Winchester, MA (US); William Haney, Wayland, MA (US); Steven O'Neil, Wayland, MA (US); Ronnie Wei, Weston, MA (US)

(21) Appl. No.: 17/769,160

(22) PCT Filed: Oct. 14, 2020
(86) PCT No.: PCT/US2020/055497

§ 371 Date: Apr. 14, 2022

Related U.S. Application Data
(60) Provisional application No. 62/915,123, filed on Oct. 15, 2019.

(51) Int. Cl. C07K 16/28

(52) U.S. Cl. CPC C07K 16/2863; C07K 16/283; C07K 16/2851; A61K 39/00; C07K 2317/24; C07K 2317/31; C07K 2317/33; C07K 2317/565; C07K 2317/622

(57) ABSTRACT

Multi-specific binding proteins that bind NKG2D receptor, CD 16, and FLT3 are described, as well as pharmaceutical compositions and therapeutic methods useful for the treatment of autoimmune disease or cancer.

This application claims priority to U.S. Provisional Application No. 62/915,123, filed on Oct. 15, 2019, the entirety of which is incorporated herein by reference.

### SEQUENCE LISTING

This application incorporates by reference in its entirety the Computer Readable Form (CRF) of a Sequence Listing in ASCII text format. The Sequence Listing text file is entitled “14247-474-888_SEQ_LISTING,” was created on Oct. 5, 2020, and is 317,572 bytes in size.

### FIELD OF THE INVENTION

The invention relates to multi-specific binding proteins that bind to NKG2D, CD16, and FLT3.

### BACKGROUND

Cancer continues to be a significant health problem despite the substantial research efforts and scientific advances reported in the literature for treating this disease. Blood and bone marrow cancers are frequently diagnosed cancer types, including multiple myelomas, leukemia, and lymphomas. Current treatment options for these cancers are not effective for all patients and/or can have substantial adverse side effects. Other types of cancer also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, e.g., WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-γ and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals. NKG2D is a type-II transmembrane protein that is expressed by essentially all natural killer cells where NKG2D serves as an activating receptor. NKG2D is also be found on T cells where it acts as a costimulatory receptor. The ability to modulate NK cell function via NKG2D is useful in various therapeutic contexts including malignancy.

Fms related tyrosine kinase 3 (FLT3), also called FLK2, STK1, or CD135, is a class III receptor tyrosine kinase. FLT3 is a transmembrane protein including multiple immunoglobulin-like domains in the extracellular region. FLT3 can be activated by binding of FLT3LG, which induces FLT3 homodimerization and autophosphorylation. Activated FLT3 subsequently phosphorylates and activates multiple cytoplasmic effector molecules such as Akt, Erk, and mTOR, thereby promoting cell proliferation and reducing apoptosis. Mutations that result in constitutive activation of FLT3 have been observed in acute myeloid leukemia and acute lymphoblastic leukemia.

### SUMMARY

The invention provides multi-specific binding proteins that bind to the NKG2D receptor and CD16 receptor on natural killer cells, and tumor-associated antigen FLT3. Such proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys. Formulations containing any one of the proteins described herein; cells containing one or more nucleic acids expressing the proteins, and methods of enhancing tumor cell death using the proteins are also provided.

Accordingly, one aspect of the invention provides a protein comprising:
(a) a first antigen-binding site that binds NKG2D;
(b) a second antigen-binding site that binds FLT3; and
(c) an antibody Fc domain or a portion thereof sufficient to bind CD16, or a third antigen-binding site that binds CD16,
   wherein the second antigen-binding site that binds FLT3 comprises:
(i) a heavy chain variable domain (VH) comprising complementarity-determining region 1 (CDR1), complementarity-determining region 2 (CDR2), and complementarity-determining region 3 (CDR3) comprising the amino acid sequences of SEQ ID NOs: 11, 4, and 55, respectively; and a light chain variable domain (VL) comprising CDR1, CDR2, and CDR3 comprising the amino acid sequences of SEQ ID NOs: 6, 7, and 8, respectively;

(ii) a VH comprising CDR1, CDR2, and CDR3 comprising the amino acid sequences of SEQ ID NOs: 59, 63, and 54, respectively; and a VL comprising CDR1, CDR2, and CDR3 comprising the amino acid sequences of SEQ ID NOs: 86, 66, and 67, respectively;

(iii) a VH comprising CDR1, CDR2, and CDR3 comprising the amino acid sequences of SEQ ID NOs: 87, 88, and 89, respectively; and a VL comprising CDR1, CDR2, and CDR3 comprising the amino acid sequences of SEQ ID NOs: 91, 92, and 93, respectively;

(iv) a VH comprising CDR1, CDR2, and CDR3 comprising the amino acid sequences of SEQ ID NOs: 97, 99, and 100, respectively; and a VL comprising CDR1, CDR2, and CDR3 comprising the amino acid sequences of SEQ ID NOs: 101, 102, and 103, respectively;

(v) a VH comprising CDR1, CDR2, and CDR3 comprising the amino acid sequences of SEQ ID NOs: 87, 98, and 89, respectively; and a VL comprising CDR1, CDR2, and CDR3 comprising the amino acid sequences of SEQ ID NOs: 106, 92, and 93, respectively;

(vi) a VH comprising CDR1, CDR2, and CDR3 comprising the amino acid sequences of SEQ ID NOs: 109, 110, and 111, respectively; and a VL comprising CDR1, CDR2, and CDR3 comprising the amino acid sequences of SEQ ID NOs: 112, 113, and 114, respectively;

(vii) a VH comprising CDR1, CDR2, and CDR3 comprising the amino acid sequences of SEQ ID NOs: 117, 118, and 119, respectively; and a VL comprising CDR1, CDR2, and CDR3 comprising the amino acid sequences of SEQ ID NOs: 120, 121, and 122, respectively;

(viii) a VH comprising CDR1, CDR2, and CDR3 comprising the amino acid sequences of SEQ ID NOs: 87, 98, and 89, respectively; and a VL comprising CDR1, CDR2, and CDR3 comprising the amino acid sequences of SEQ ID NOs: 106, 92, and 93, respectively;

(ix) a VH comprising CDR1, CDR2, and CDR3 comprising the amino acid sequences of SEQ ID NOs: 62, 33, and 127, respectively; and a VL comprising CDR1, CDR2, and CDR3 comprising the amino acid sequences of SEQ ID NOs: 128, 129, and 130, respectively; or

(x) a VH comprising CDR1, CDR2, and CDR3 comprising the amino acid sequences of SEQ ID NOs: 132, 133, and 134, respectively; and a VL comprising CDR1, CDR2, and CDR3 comprising the amino acid sequences of SEQ ID NOs: 65, 66, and 46, respectively.

In certain embodiments, the second antigen-binding site that binds FLT3 comprises a VH comprising CDR1, CDR2, and CDR3 comprising the amino acid sequences of SEQ ID NOs: 11, 4, and 55, respectively; and a VL comprising CDR1, CDR2, and CDR3 comprising the amino acid sequences of SEQ ID NOs: 6, 7, and 8, respectively. In certain embodiments, the second antigen-binding site that binds FLT3 comprises a VH comprising CDR1, CDR2, and CDR3 comprising the amino acid sequences of SEQ ID NOs: 11, 4, and 5, respectively; and a VL comprising CDR1, CDR2, and CDR3 comprising the amino acid sequences of SEQ ID NOs: 6, 7, and 8, respectively. In certain embodiments, the second antigen-binding site that binds FLT3 comprises a VH comprising CDR1, CDR2, and CDR3 comprising the amino acid sequences of SEQ ID NOs: 11, 4, and 50, respectively; and a VL comprising CDR1, CDR2, and CDR3 comprising the amino acid sequences of SEQ ID NOs: 6, 7, and 8, respectively. In certain embodiments, the VH comprises an amino acid sequence at least 90% identical to SEQ ID NO:37, and the VL comprises an amino acid sequence at least 90% identical to SEQ ID NO:38. In certain embodiments, the VH comprises the amino acid sequence of SEQ ID NO:53, and the VL comprises the amino acid sequence of SEQ ID NO:42. In certain embodiments, the VH and the VL comprise the amino acid sequences of SEQ ID NOs: 9 and 10; 13 and 10; 17 and 10; 9 and 22; 9 and 26; 9 and 30; 9 and 34; 37 and 38; 41 and 42; 45 and 42; or 49 and 42, respectively. In certain embodiments, the second antigen-binding site is present as a single-chain fragment variable (scFv), and wherein the scFv comprises an amino acid sequence selected from SEQ ID NOs: 3, 12, 15, 16, 19, 20, 23, 24, 27, 28, 31, 32, 35, 36, 39, 40, 43, 44, 47, 48, 51, and 52.

In certain embodiments, the second antigen-binding site that binds FLT3 comprises a VH comprising CDR1, CDR2, and CDR3 comprising the amino acid sequences of SEQ ID NOs: 59, 63, and 54, respectively; and a VL comprising CDR1, CDR2, and CDR3 comprising the amino acid sequences of SEQ ID NOs: 86, 66, and 67, respectively. In certain embodiments, the second antigen-binding site that binds FLT3 comprises a VH comprising CDR1, CDR2, and CDR3 comprising the amino acid sequences of SEQ ID NOs: 78, 63, 79, respectively; and a VL comprising CDR1, CDR2, and CDR3 comprising the amino acid sequences of SEQ ID NOs: 80, 66, 67, respectively. In certain embodiments, the second antigen-binding site that binds FLT3 comprises a VH comprising CDR1, CDR2, and CDR3 comprising the amino acid sequences of SEQ ID NOs: 62, 63, 64, respectively; and a VL comprising CDR1, CDR2, and CDR3 comprising the amino acid sequences of SEQ ID NOs: 65, 66, 67, respectively. In certain embodiments, the VH comprises an amino acid sequence at least 90% identical to SEQ ID NO:76, and the VL comprises an amino acid sequence at least 90% identical to SEQ ID NO:77. In certain embodiments, the VH comprises the amino acid sequence of SEQ ID NO:29, and the VL comprises the amino acid sequence of SEQ ID NO:84. In certain embodiments, the VH and the VL comprise the amino acid sequences of SEQ ID NOs: 68 and 69; 72 and 73; or 76 and 77, respectively. In certain embodiments, the second antigen-binding site is present as an scFv, and wherein the scFv comprises an amino acid sequence selected from SEQ ID NOs: 70, 71, 74, 75, 81, and 82.

In certain embodiments, the second antigen-binding site that binds FLT3 comprises a VH comprising CDR1, CDR2, and CDR3 comprising the amino acid sequences of SEQ ID NOs: 87, 88, and 89, respectively; and a VL comprising CDR1, CDR2, and CDR3 comprising the amino acid sequences of SEQ ID NOs: 91, 92, and 93, respectively.

In certain embodiments, the second antigen-binding site that binds FLT3 comprises a VH comprising CDR1, CDR2, and CDR3 comprising the amino acid sequences of SEQ ID NOs: 97, 99, and 100, respectively; and a VL comprising CDR1, CDR2, and CDR3 comprising the amino acid sequences of SEQ ID NOs: 101, 102, and 103, respectively.

In certain embodiments, the second antigen-binding site that binds FLT3 comprises a VH comprising CDR1, CDR2, and CDR3 comprising the amino acid sequences of SEQ ID NOs: 87, 98, and 89, respectively; and a VL comprising CDR1, CDR2, and CDR3 comprising the amino acid sequences of SEQ ID NOs: 106, 92, and 93, respectively.

In certain embodiments, the second antigen-binding site that binds FLT3 comprises a VH comprising CDR1, CDR2, and CDR3 comprising the amino acid sequences of SEQ ID NOs: 109, 110, and 111, respectively; and a VL comprising CDR1, CDR2, and CDR3 comprising the amino acid sequences of SEQ ID NOs: 112, 113, and 114, respectively.

In certain embodiments, the second antigen-binding site that binds FLT3 comprises a VH comprising CDR1, CDR2, and CDR3 comprising the amino acid sequences of SEQ ID NOs: 117, 118, and 119, respectively; and a VL comprising CDR1, CDR2, and CDR3 comprising the amino acid sequences of SEQ ID NOs: 120, 121, and 122, respectively.

In certain embodiments, the second antigen-binding site that binds FLT3 comprises a VH comprising CDR1, CDR2, and CDR3 comprising the amino acid sequences of SEQ ID NOs: 87, 98, and 89, respectively; and a VL comprising CDR1, CDR2, and CDR3 comprising the amino acid sequences of SEQ ID NOs: 106, 92, and 93, respectively.

In certain embodiments, the second antigen-binding site that binds FLT3 comprises a VH comprising CDR1, CDR2, and CDR3 comprising the amino acid sequences of SEQ ID NOs: 62, 33, and 127, respectively; and a VL comprising CDR1, CDR2, and CDR3 comprising the amino acid sequences of SEQ ID NOs: 128, 129, and 130, respectively.

In certain embodiments, the second antigen-binding site that binds FLT3 comprises a VH comprising CDR1, CDR2, and CDR3 comprising the amino acid sequences of SEQ ID NOs: 132, 133, and 134, respectively; and a VL comprising CDR1, CDR2, and CDR3 comprising the amino acid sequences of SEQ ID NOs: 65, 66, and 46, respectively.

In certain embodiments, the second antigen-binding site binds human FLT3 with a dissociation constant (K_{D}) smaller than or equal to 20 nM as measured by surface plasmon resonance (SPR). In certain embodiments, the second antigen-binding site binds human FLT3 with a K_{D} smaller than or equal to 10 nM as measured by SPR. In certain embodiments, the second antigen-binding site binds cynomolgus FLT3. In certain embodiments, the second antigen-binding site does not compete with FLT3L for binding FLT3.

In certain embodiments, the protein comprises an antibody Fc domain or a portion thereof sufficient to bind CD16.

In certain embodiments, the first antigen-binding site that binds NKG2D is an Fab fragment, and the second antigen-binding site that binds FLT3 is an scFv. In certain embodiments, the first antigen-binding site that binds NKG2D is an scFv, and the second antigen-binding site that binds FLT3 is an Fab fragment.

In certain embodiments, the protein further comprises an additional antigen-binding site that binds FLT3. In certain embodiments, the first antigen-binding site that binds NKG2D is an scFv, and the second and the additional antigen-binding sites that bind FLT3 are each an Fab fragment. In certain embodiments, the first antigen-binding site that binds NKG2D is an scFv, and the second and the additional antigen-binding sites that bind FLT3 are each an scFv.

In certain embodiments, the scFv that binds FLT3 and/or the scFv that binds NKG2D comprise a heavy chain variable domain and a light chain variable domain. In certain embodiments, the scFv is linked to an antibody constant domain or a portion thereof sufficient to bind CD16, via a hinge comprising Ala-Ser or Gly-Ser. In certain embodiments, the hinge further comprises amino acid sequence Thr-Lys-Gly. In certain embodiments, the heavy chain variable domain of the scFv forms a disulfide bridge with the light chain variable domain of the scFv. In certain embodiments, the disulfide bridge is formed between C44 of the heavy chain variable domain and C100 of the light chain variable domain, numbered under the Kabat numbering scheme. In certain embodiments, the heavy chain variable domain of the scFv is linked to the light chain variable domain of the scFv via a flexible linker. In certain embodiments, the flexible linker comprises (G₄S)₄. In certain embodiments, within the scFv the heavy chain variable domain is positioned at the C-terminus of the light chain variable domain. In certain embodiments, within the scFv the heavy chain variable domain is positioned at the N-terminus of the light chain variable domain.

In certain embodiments, the Fab is not positioned between an antigen-binding site and the Fc or the portion thereof.

In certain embodiments, the first antigen-binding site that binds NKG2D comprises a VH comprising CDR1, CDR2, and CDR3 comprising the amino acid sequences of SEQ ID NOs: 240 or 241, 242, and 270 or 271, respectively; and a VL comprising CDR1, CDR2, and CDR3 comprising the amino acid sequences of SEQ ID NOs: 276, 236, and 245, respectively.

In certain embodiments, the first antigen-binding site that binds NKG2D comprises:
(i) a VH comprising CDR1, CDR2, and CDR3 comprising the amino acid sequences of SEQ ID NOs: 240 or 241, 242, and 255 or 256, respectively; and a VL comprising CDR1, CDR2, and CDR3 comprising the amino acid sequences of SEQ ID NOs:276, 236, and 245, respectively; or
(ii) a VH comprising CDR1, CDR2, and CDR3 comprising the amino acid sequences of SEQ ID NOs: 240 or 241, 242, and 243 or 244, respectively; and a VL comprising CDR1, CDR2, and CDR3 comprising the amino acid sequences of SEQ ID NOs: 276, 236, and 245, respectively.

In certain embodiments, the VH of the first antigen-binding site comprises an amino acid sequence at least 90% identical to SEQ ID NO:254, and the VL of the first antigen-binding site comprises an amino acid sequence at least 90% identical to SEQ ID NO:239. In certain embodiments, the VH of the first antigen-binding site comprises the amino acid sequence of SEQ ID NO:254, and the VL of the first antigen-binding site comprises the amino acid sequence of SEQ ID NO:239.

In certain embodiments, the antibody Fc domain is a human IgG1 antibody Fc domain. In certain embodiments, the antibody Fc domain or the portion thereof comprises an amino acid sequence at least 90% identical to SEQ ID NO:136.

In certain embodiments, at least one polypeptide chain of the antibody Fc domain comprises one or more mutations, relative to SEQ ID NO:136, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system. In certain embodiments, at least one polypeptide chain of the antibody Fc domain comprises one or more mutations, relative to SEQ ID NO:136, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system. In certain embodiments, one polypeptide chain of the antibody heavy chain constant region comprises one or more mutations, relative to SEQ ID NO:136, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:136, at one or more positions selected from Q347, Y349, L351, S354, E356, E357, S364, T366, L368, K370, N390, K392, T394, D399, D401, F405, Y407, K409, T411, and K439, numbered according to the EU numbering system. In certain embodiments, one polypeptide chain of the antibody heavy chain constant region comprises K360E and K409W substitutions relative to SEQ ID NO:136; and the other polypeptide chain of the antibody heavy chain constant region comprises Q347R, D399V and F405T substitutions relative to SEQ ID NO:136, numbered according to the EU numbering system. In certain embodiments, one polypeptide chain of the antibody heavy chain constant region comprises a Y349C substitution relative to SEQ ID NO:136; and the other polypeptide chain of the antibody heavy chain constant region comprises an S354C substitution relative to SEQ ID NO:136, numbered according to the EU numbering system.

In another aspect, the present invention provides a pharmaceutical composition comprising a protein disclosed herein and a pharmaceutically acceptable carrier.

In another aspect, the present invention provides a cell comprising one or more nucleic acids encoding a protein disclosed herein.

In another aspect, the present invention provides a method of enhancing tumor cell death, the method comprising exposing the tumor cell and a natural killer cell to an effective amount of the protein or pharmaceutical composition disclosed herein.

In another aspect, the present invention provides a method of treating cancer, the method comprising administering an effective amount of the protein or pharmaceutical composition disclosed herein to a patient in need thereof.

In certain embodiments, the cancer is a hematologic malignancy. In certain embodiments, the hematologic malignancy is leukemia. In certain embodiments, selected from the group consisting of acute myeloid leukemia (AML), acute lymphoblastic leukemia (ALL), myelodysplasia, acute T-lymphoblastic leukemia, and acute promyelocytic leukemia. In certain embodiments, the cancer expresses FLT3.

Various aspects and embodiments of the invention are described in further detail below.

### BRIEF DESCRIPTION OF THE DRAWINGS

FIG. 1 is a representation of a heterodimeric, multi-specific antibody, e.g., a trispecific binding protein (TriNKET). Each arm can represent either the NKG2D-binding domain, or the binding domain corresponding to FLT3. In some embodiments, the NKG2D binding domain and the FLT3 binding domains can share a common light chain.

FIGS. 2A-2E illustrate five exemplary formats of a multi-specific binding protein, e.g., a trispecific binding protein (TriNKET). As shown in FIG. 2A, either the NKG2D-binding domain or the FLT3 binding domain can take the scFv format (left arm). An antibody that contains a NKG2D-targeting scFv, a FLT3-targeting Fab fragment, and a heterodimerized antibody constant region is referred herein as the F3-TriNKET. An antibody that contains a FLT3-targeting scFv, a NKG2D-targeting Fab fragment, and a heterodimerized antibody constant region/domain that binds CD16 is referred herein as the F3′-TriNKET (FIG. 2E). As shown in FIG. 2B, both the NKG2D-binding domain and FLT3-binding domain can take the scFv format. FIGS. 2C to 2D are illustrations of an antibody with three antigen-binding sites, including two antigen-binding sites that bind FLT3, and the NKG2D-binding site fused to the heterodimerized antibody constant region. These antibody formats are referred herein as F4-TriNKET. FIG. 2C illustrates that the two FLT3-binding sites are in the Fab fragment format, and the NKG2D binding site in the scFv format. FIG. 2D illustrates that the FLT3 binding sites are in the scFv format, and the NKG2D binding site is in the scFv format. FIG. 2E represents a trispecific antibody (TriNKET) that contains a tumor-targeting scFv, a NKG2D-targeting Fab fragment, and a heterodimerized antibody constant region/domain (“CD domain”) that binds CD16. The antibody format is referred herein as F3′-TriNKET. In certain exemplary multispecific binding proteins, heterodimerization mutations on the antibody constant region include K360E and K409W on one constant domain; and Q347R, D399V and F405T on the opposite constant domain (shown as a triangular lock-and-key shape in the CD domains). The bold bar between the heavy and the light chain variable domains of the Fab fragments represents a disulfide bond.

FIG. 3 is a representation of a TriNKET in the Triomab form, which is a trifunctional, bispecific antibody that maintains an IgG-like shape. This chimera consists of two half antibodies, each with one light and one heavy chain, that originate from two parental antibodies. Triomab form may be a heterodimeric construct containing ½ of rat antibody and ½ of mouse antibody.

FIG. 4 is a representation of a TriNKET in the KiH Common Light Chain form, which involves the knobs-into-holes (KIHs) technology. KiH is a heterodimer containing 2 Fab fragments binding to target 1 and 2, and an Fc stabilized by heterodimerization mutations. TriNKET in the KiH format may be a heterodimeric construct with 2 Fab fragments binding to target 1 and target 2, containing two different heavy chains and a common light chain that pairs with both heavy chains.

FIG. 5 is a representation of a TriNKET in the dual-variable domain immunoglobulin (DVD-Ig™) form, which combines the target-binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of a variable domain of Fab fragment targeting antigen 1. DVD-Ig™ form contains normal Fc.

FIG. 6 is a representation of a TriNKET in the Orthogonal Fab fragment interface (Ortho-Fab) form, which is a heterodimeric construct that contains 2 Fab fragments binding to target 1 and target 2 fused to Fc. Light chain (LC)-heavy chain (HC) pairing is ensured by orthogonal interface. Heterodimerization is ensured by mutations in the Fc.

FIG. 7 is a representation of a TriNKET in the 2-in-1 Ig format.

FIG. 8 is a representation of a TriNKET in the ES form, which is a heterodimeric construct containing two different Fab fragments binding to target 1 and target 2 fused to the Fc. Heterodimerization is ensured by electrostatic steering mutations in the Fc.

FIG. 9 is a representation of a TriNKET in the Fab Arm Exchange form: antibodies that exchange Fab fragment arms by swapping a heavy chain and attached light chain (half-molecule) with a heavy-light chain pair from another molecule, resulting in bispecific antibodies. Fab Arm Exchange form (cFae) is a heterodimer containing 2 Fab fragments binding to target 1 and 2, and an Fc stabilized by heterodimerization mutations.

FIG. 10 is a representation of a TriNKET in the SEED Body form, which is a heterodimer containing 2 Fab fragments binding to target 1 and 2, and an Fc stabilized by heterodimerization mutations.

FIG. 11 is a representation of a TriNKET in the LuZ-Y form, in which a leucine zipper is used to induce heterodimerization of two different HCs. The LuZ-Y form is a heterodimer containing two different scFabs binding to target 1 and 2, fused to Fc. Heterodimerization is ensured through leucine zipper motifs fused to C-terminus of Fc.

FIG. 12 is a representation of a TriNKET in the Cov-X-Body form.

FIGS. 13A-13B are representations of TriNKETs in the κλ-Body forms, which are heterodimeric constructs with two different Fab fragments fused to Fc stabilized by heterodimerization mutations: one Fab fragment targeting antigen 1 contains kappa LC, and the second Fab fragment targeting antigen 2 contains lambda LC. FIG. 13A is an exemplary representation of one form of a κλ-Body; FIG. 13B is an exemplary representation of another κλ-Body.

FIG. 14 is an Oasc-Fab heterodimeric construct that includes Fab fragment binding to target 1 and scFab binding to target 2, both of which are fused to the Fc domain. Heterodimerization is ensured by mutations in the Fc domain.

FIG. 15 is a DuetMab, which is a heterodimeric construct containing two different Fab fragments binding to antigens 1 and 2, and an Fc that is stabilized by heterodimerization mutations. Fab fragments 1 and 2 contain differential S-S bridges that ensure correct light chain and heavy chain pairing.

FIG. 16 is a CrossmAb, which is a heterodimeric construct with two different Fab fragments binding to targets 1 and 2, and an Fc stabilized by heterodimerization mutations. CL and CH1 domains, and VH and VL domains are switched, e.g., CH1 is fused in-line with VL, while CL is fused in-line with VH.

FIG. 17 is a Fit-Ig, which is a homodimeric construct where Fab fragment binding to antigen 2 is fused to the N-terminus of HC of Fab fragment that binds to antigen 1. The construct contains wild-type Fc.

FIG. 18 is a set of sensograms showing SPR profiles of antibodies collected from the murine hybridomas supernatants binding to hFLT3.

FIG. 19 is a set of sensograms showing SPR profiles of antibodies collected from the murine mAb subclones binding to hFLT3.

FIG. 20 is a bar graph depicting the the reduction of the ability of the candidate antibodies to bind FLT3-expressing EOL-1 cancer cells by saturating concentrations of soluble FLT3-ligand.

FIGS. 21A-21C are line graphs showing binding of FLT3-targeting TriNKET F3′-1158 and its parental monoclonal antibody to FLT3-expressing cell lines RMA-hFLT3 (FIG. 21A), RMA-cFLT3 (FIG. 21B), and REH (FIG. 21C).

FIGS. 22A-22B are line graphs showing binding of FLT3-targeted TriNKET F3′-1158 and its parental monoclonal antibody to MOLM-13 cells, which expressed FLT3 with T227M mutation (FIG. 22A), and MV4-11 cells, which expressed FLT2 with the ITD mutation.

FIGS. 23A-23B are line graphs showing internalization of FLT3-targeted TriNKET F3′-1158 and its parental monoclonal antibody into FLT3-expressing cell lines REH (FIG. 23A) and EOL-1 (FIG. 23B).

FIGS. 24A-24D are bar graphs showing NK cell-mediated lysis of FLT3-expressing cancer cell lines EOL-1 (FIG. 24A), REH (FIG. 24B), RS4-11 (FIG. 24C), and MV4-11 (FIG. 24D) in the presence of TriNKET F3′-1158 and its parental monoclonal antibody.

FIG. 25 is a line graph depicting NK cell-mediated lysis of FLT3-expressing cancer cell line REH in the presence of TriNKET F3′-1158, a NKG2D-dead variant thereof (“F3′-1158 dead-2D”), an Fc-silent variant thereof (“F3′-1158si”), or its parental monoclonal antibody 1158 mAb.

FIG. 26 is a line graph showing CD8 T-cell mediated lysis of FLT3-expressing acute lymphoblastic leukemia cell line RS4-11 in the presence of TriNKET F3′-1158 and its parental monoclonal antibody.

FIG. 27 is a set of histograms showing binding of TriNKET F3′-1158 and its parental monoclonal antibody to blood cells.

FIGS. 28A-28B are bar graphs showing FLT3 phosphorylation by TriNKET F3′-1158 and its parental monoclonal antibody in the absence (FIG. 28A) or presence (FIG. 28B) of FLT3-ligand. The FLT3-ligand sample in FIG. 7A serves as a positive control.

### DETAILED DESCRIPTION

The invention provides multi-specific binding proteins that bind the NKG2D receptor and CD16 receptor on natural killer cells, and tumor-associated antigen FLT3. In some embodiments, the multi-specific proteins further include an additional antigen-binding site that binds FLT3. The invention also provides pharmaceutical compositions comprising such multi-specific binding proteins, and therapeutic methods using such multi-specific proteins and pharmaceutical compositions, for purposes such as treating autoimmune diseases and cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The term “tumor-associated antigen” as used herein means any antigen including but not limited to a protein, glycoprotein, ganglioside, carbohydrate, lipid that is associated with cancer. Such antigen can be expressed on malignant cells or in the tumor microenvironment such as on tumor-associated blood vessels, extracellular matrix, mesenchymal stroma, or immune infiltrates.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecyl sulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “FLT3” (also known as FLK2, STK1, or CD135) refers to the protein of Uniprot Accession No. P36888 and related isoforms.

As used herein, “FLT3L” (also known as FLT3-ligand) refers to the protein of Uniprot Accession No. P49771 and related isoforms.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Proteins

The invention provides multi-specific binding proteins that bind to the NKG2D receptor and CD16 receptor on natural killer cells, and tumor-associated antigen FLT3. The multi-specific binding proteins are useful in the pharmaceutical compositions and therapeutic methods described herein. Binding of the multi-specific binding proteins to the NKG2D receptor and CD16 receptor on a natural killer cell enhances the activity of the natural killer cell toward destruction of tumor cells expressing FLT3. Binding of the multi-specific binding proteins to FLT3-expressing tumor cells brings these cells into proximity with the natural killer cell, which facilitates direct and indirect destruction of the tumor cells by the natural killer cell. Multi-specific binding proteins that bind NKG2D, CD16, and another target are disclosed in International Application Publication Nos. WO2018148445 and WO2019157366, which are not incorporated herein by reference. Further description of some exemplary multi-specific binding proteins is provided below.

The first component of the multi-specific binding protein is an antigen-binding site that binds to NKG2D receptor-expressing cells, which can include but are not limited to NK cells, γδ T cells and CD8⁺αβ T cells. Upon NKG2D binding, the multi-specific binding proteins may block natural ligands, such as ULBP6 and MICA, from binding to NKG2D and activating NK cells.

The second component of the multi-specific binding proteins is an antigen-binding site that binds FLT3. FLT3-expressing cells may be found, for example, in acute myeloid leukemia (AML) and acute lymphoblastic leukemia (ALL). FLT3-expressing cells may be found in association with other cancers and tumor types, for example, hematologic malignancies, leukemia, acute myeloid leukemia (AML), acute lymphoblastic leukemia (ALL), myelodysplasia, acute T-lymphoblastic leukemia, and acute promyelocytic leukemia.

The third component of the multi-specific binding proteins is an antibody Fc domain or a portion thereof or an antigen-binding site that binds to cells expressing CD16, an Fc receptor on the surface of leukocytes including natural killer cells, macrophages, neutrophils, eosinophils, mast cells, and follicular dendritic cells.

An additional antigen-binding site of the multi-specific binding proteins may bind FLT3. In certain embodiments, the first antigen-binding site that binds NKG2D is an scFv, and the second and the additional antigen-binding sites bind FLT3, which are each an Fab fragment. In certain embodiments, the first antigen-binding site that binds NKG2D is an scFv, and the second and the additional antigen-binding sites binds FLT3, which are each an scFv.

The antigen-binding sites may each incorporate an antibody heavy chain variable domain and an antibody light chain variable domain (e.g., arranged as in an antibody, or fused together to from an scFv), or one or more of the antigen-binding sites may be a single domain antibody, such as a V_{H}H antibody like a camelid antibody or a V_{NAR} antibody like those found in cartilaginous fish.

In some embodiments, the second antigen-binding site incorporates a light chain variable domain having an amino acid sequence identical to the amino acid sequence of the light chain variable domain present in the first antigen-binding site.

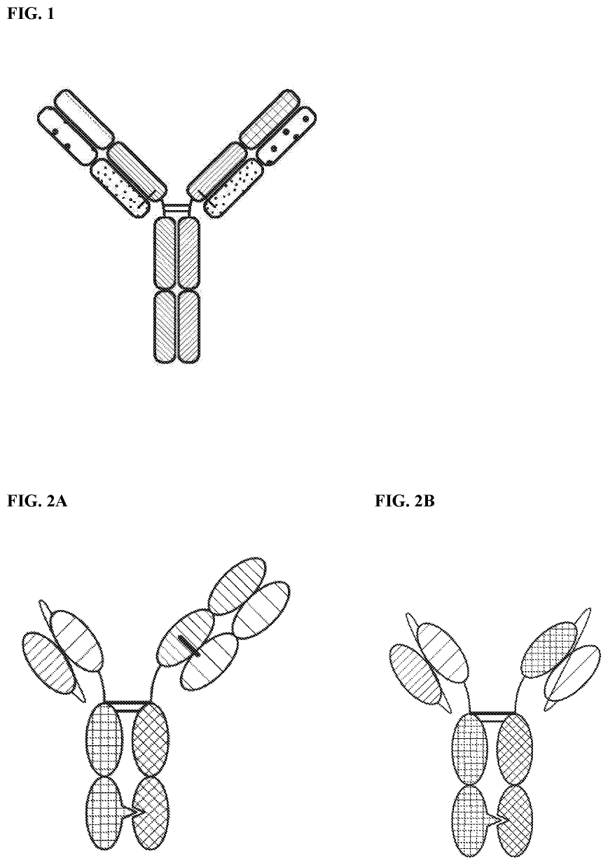

The multi-specific binding proteins described herein can take various formats. For example, one format is a heterodimeric, multi-specific antibody including a first immunoglobulin heavy chain, a first immunoglobulin light chain, a second immunoglobulin heavy chain and a second immunoglobulin light chain (FIG. 1). The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and optionally a first light chain constant domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and optionally a second light chain constant domain. The second immunoglobulin light chain, together with the second immunoglobulin heavy chain, forms an antigen-binding site that binds FLT3. The first Fc domain and second Fc domain together are able to bind to CD16 (FIG. 1). In some embodiments, the first immunoglobulin light chain is identical to the second immunoglobulin light chain.

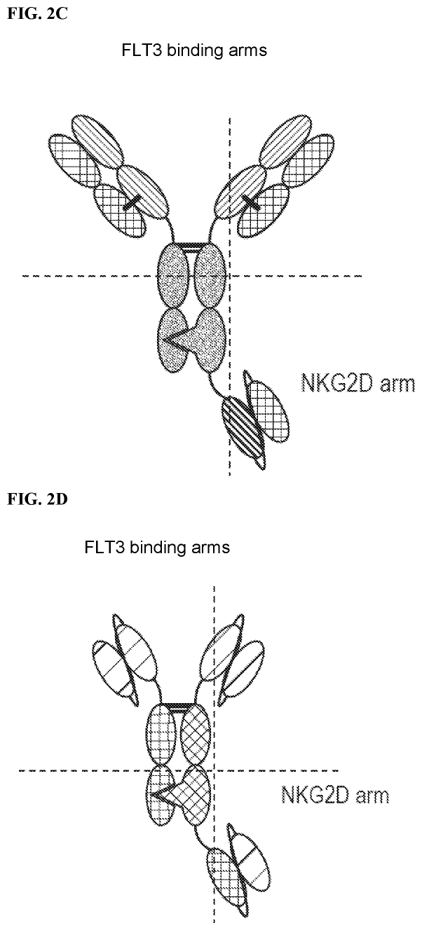

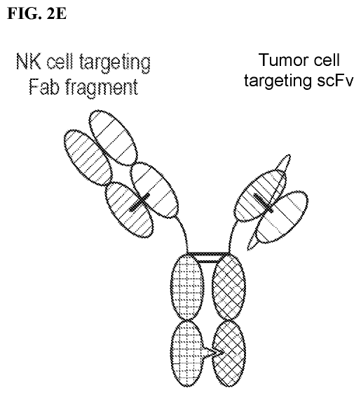

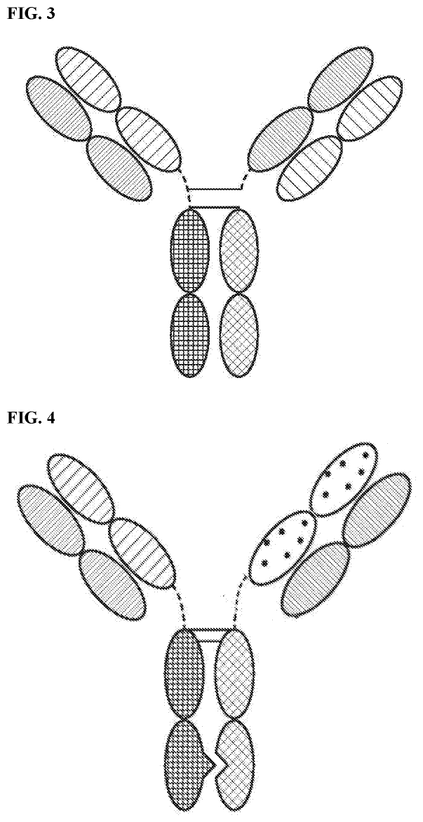

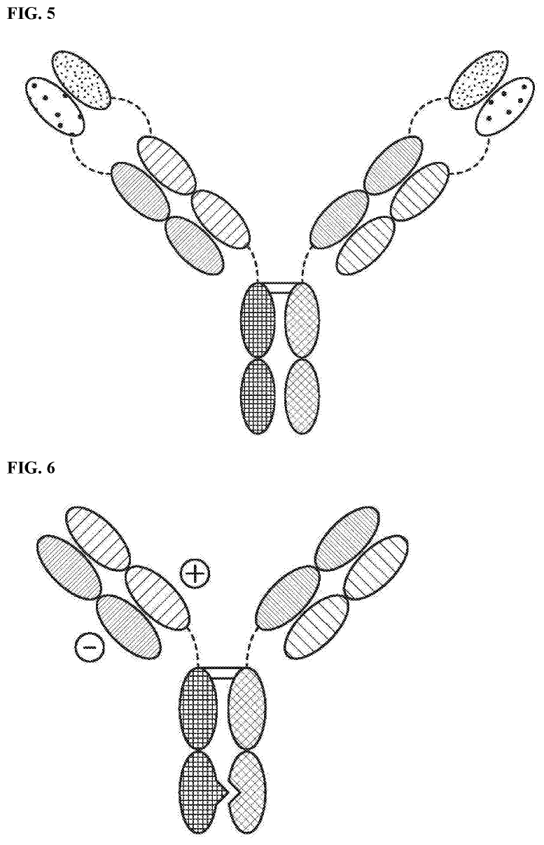

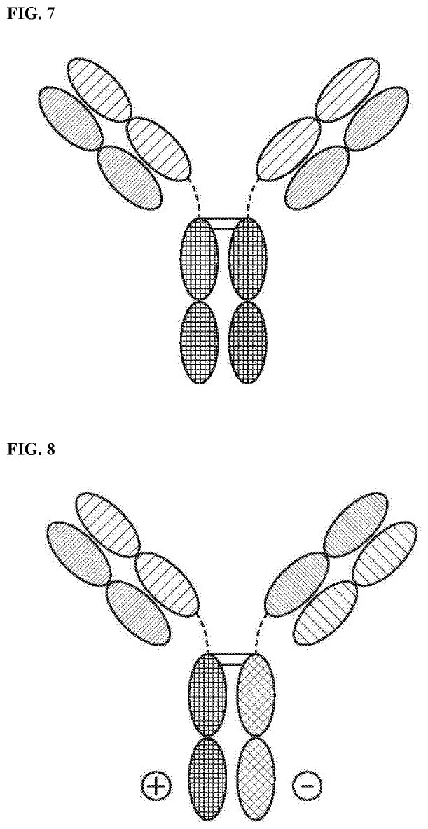

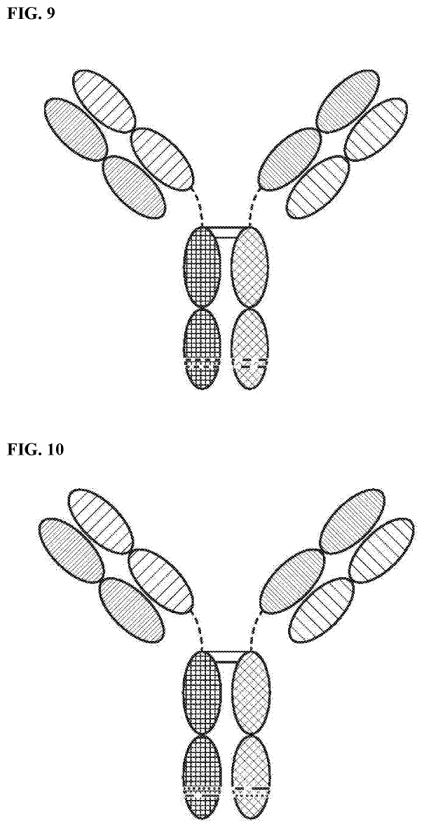

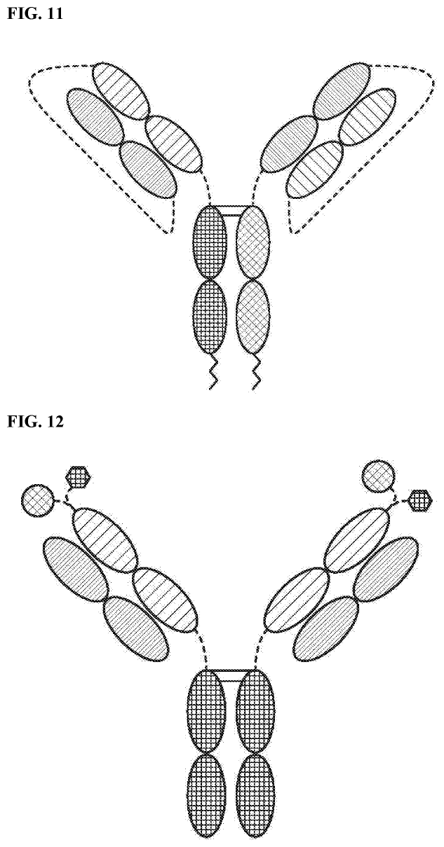

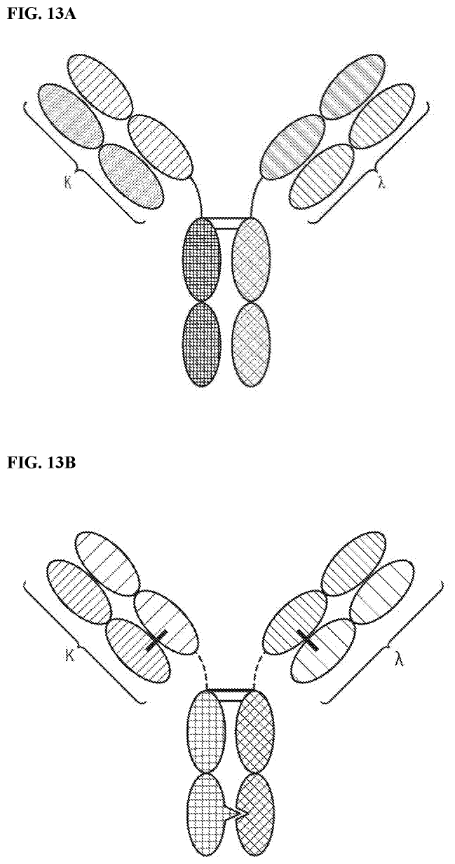

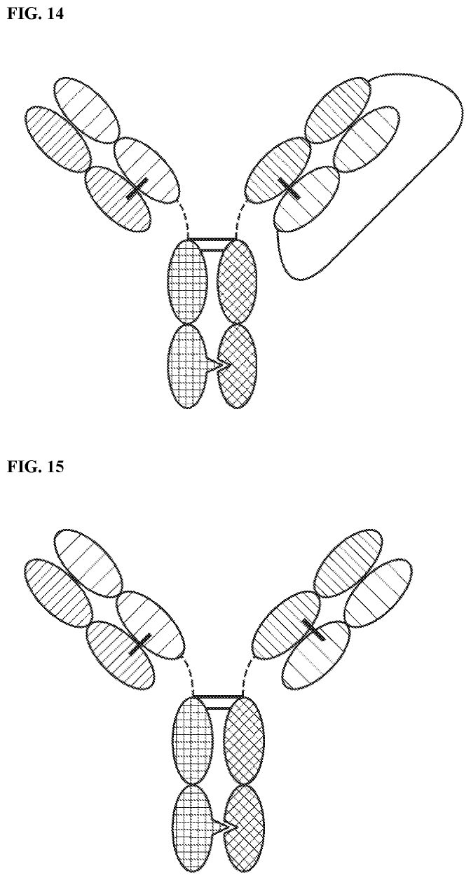

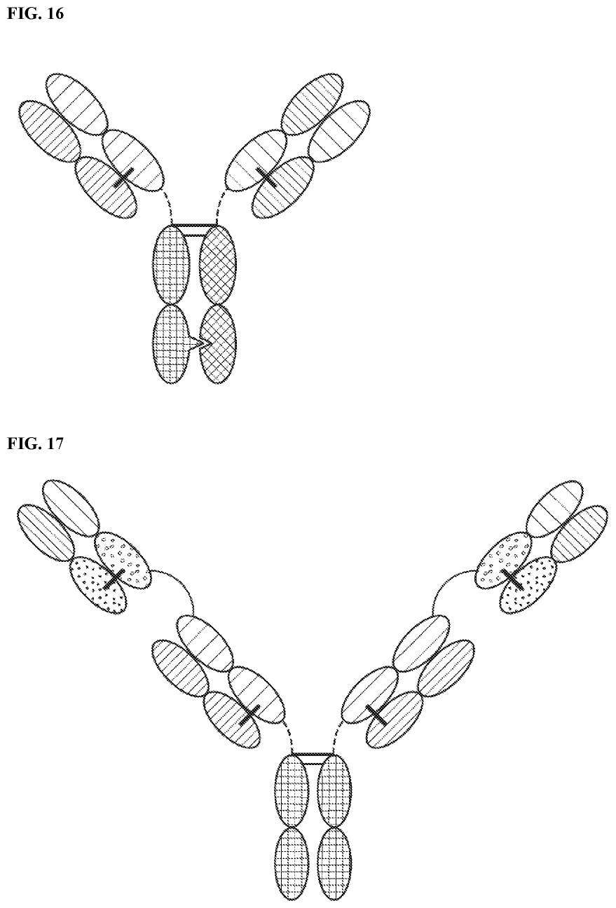

Another exemplary format involves a heterodimeric, multi-specific antibody including a first immunoglobulin heavy chain, a second immunoglobulin heavy chain and an immunoglobulin light chain (FIG. 2A). The first immunoglobulin heavy chain includes a first Fc (hinge-CH2-CH3) domain fused via either a linker or an antibody hinge to a single-chain variable fragment (scFv) composed of a heavy chain variable domain and light chain variable domain which pair and bind NKG2D, or bind FLT3. The second immunoglobulin heavy chain includes a second Fc (hinge-CH2-CH3) domain, a second heavy chain variable domain and a CH1 heavy chain domain. The immunoglobulin light chain includes a light chain variable domain and a light chain constant domain. The second immunoglobulin heavy chain pairs with the immunoglobulin light chain and binds to NKG2D or binds FLT3. The first Fc domain and the second Fc domain together are able to bind to CD16 (FIG. 2A).

Another exemplary format involves a heterodimeric, multi-specific antibody including a first immunoglobulin heavy chain, and a second immunoglobulin heavy chain (FIG. 2B). The first immunoglobulin heavy chain includes a first Fc (hinge-CH2-CH3) domain fused via either a linker or an antibody hinge to a single-chain variable fragment (scFv) composed of a heavy chain variable domain and light chain variable domain which pair and bind NKG2D, or bind FLT3. The second immunoglobulin heavy chain includes a second Fc (hinge-CH2-CH3) domain fused via either a linker or an antibody hinge to a single-chain variable fragment (scFv) composed of a heavy chain variable domain and light chain variable domain which pair and bind NKG2D, or bind FLT3. The first Fc domain and the second Fc domain together are able to bind to CD16 (FIG. 2B).

In some embodiments, the single-chain variable fragment (scFv) described above is linked to the antibody constant domain via a hinge sequence. In some embodiments, the hinge comprises amino acids Ala-Ser or Gly-Ser. In some embodiments, the hinge connecting an scFv that binds NKG2D and the antibody heavy chain constant domain comprises amino acids Ala-Ser. In some embodiments, the hinge connecting an scFv that binds FLT3 and the antibody heavy chain constant domain comprises amino acids Gly-Ser. In some other embodiments, the hinge comprises amino acids Ala-Ser and Thr-Lys-Gly. The hinge sequence can provide flexibility of binding to the target antigen, and balance between flexibility and optimal geometry.

In some embodiments, the single-chain variable fragment (scFv) described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat. In some embodiments, the heavy chain variable domain is linked to the light chain variable domain via a flexible linker. Any suitable linker can be used, for example, the (G₄S)₄ linker. In some embodiments of the scFv, the heavy chain variable domain is positioned at the N-terminus of the light chain variable domain. In some embodiments of the scFv, the heavy chain variable domain is positioned at the C terminus of the light chain variable domain.

The multi-specific binding proteins described herein can further include one or more additional antigen-binding sites. The additional antigen-binding site(s) may be fused to the N-terminus of the constant region CH2 domain or to the C-terminus of the constant region CH3 domain, optionally via a linker sequence. In certain embodiments, the additional antigen-binding site(s) takes the form of a single-chain variable region (scFv) that is optionally disulfide-stabilized, resulting in a tetravalent or trivalent multispecific binding protein. For example, a multi-specific binding protein includes a first antigen-binding site that binds NKG2D, a second antigen-binding site that binds FLT3, an additional antigen-binding site that binds FLT3, and an antibody constant region or a portion thereof sufficient to bind CD16 or a fourth antigen-binding site that binds CD16. Any one of these antigen binding sites can either take the form of an Fab fragment or an scFv, such as the scFv described above.

In some embodiments, the additional antigen-binding site binds a different epitope of FLT3 from the second antigen-binding site. In some embodiments, the additional antigen-binding site binds the same epitope as the second antigen-binding site. In some embodiments, the additional antigen-binding site comprises the same heavy chain and light chain CDR sequences as the second antigen-binding site. In some embodiments, the additional antigen-binding site comprises the same heavy chain and light chain variable domain sequences as the second antigen-binding site. In some embodiments, the additional antigen-binding site has the same amino acid sequence(s) as the second antigen-binding site. Exemplary formats are shown in FIGS. 2C and 2D. Accordingly, the multi-specific binding proteins can provide bivalent engagement of FLT3. Bivalent engagement of FLT3 by the multi-specific proteins can stabilize FLT3 on the tumor cell surface and enhance cytotoxicity of NK cells towards the tumor cells. Bivalent engagement of FLT3 by the multi-specific proteins can confer stronger binding of the multi-specific proteins to the tumor cells, thereby facilitating stronger cytotoxic response of NK cells towards the tumor cells, especially towards tumor cells expressing a low level of FLT3.

The multi-specific binding proteins can take additional formats. In some embodiments, the multi-specific binding protein is in the Triomab form, which is a trifunctional, bispecific antibody that maintains an IgG-like shape. This chimera consists of two half antibodies, each with one light and one heavy chain, that originate from two parental antibodies.

In some embodiments, the multi-specific binding protein is the KiHform, which involves the knobs-into-holes (KiHs) technology. The KiH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcγRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in the dual-variable domain immunoglobulin (DVD-Ig™) form, which combines the target binding domains of two monoclonal antibodies via flexible naturally occurring linkers, and yields a tetravalent IgG-like molecule.

In some embodiments, the multi-specific binding protein is in the Orthogonal Fab interface (Ortho-Fab) form. In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab fragment, without any changes being made to the other Fab fragment.

In some embodiments, the multi-specific binding protein is in the 2-in-1 Ig format. In some embodiments, the multi-specific binding protein is in the ES form, which is a heterodimeric construct containing two different Fab fragments binding to targets 1 and target 2 fused to the Fc. Heterodimerization is ensured by electrostatic steering mutations in the Fc.

In some embodiments, the multi-specific binding protein is in the κλ-Body form, which is a heterodimeric construct with two different Fab fragments fused to Fc stabilized by heterodimerization mutations: Fab fragment1 targeting antigen 1 contains kappa LC, while second Fab fragment targeting antigen 2 contains lambda LC. FIG. 13A is an exemplary representation of one form of a κλ-Body; FIG. 13B is an exemplary representation of another κλ-Body.

In some embodiments, the multi-specific binding protein is in Fab Arm Exchange form (antibodies that exchange Fab fragment arms by swapping a heavy chain and attached light chain (half-molecule) with a heavy-light chain pair from another molecule, which results in bispecific antibodies).

In some embodiments, the multi-specific binding protein is in the SEED Body form. The strand-exchange engineered domain (SEED) platform was designed to generate asymmetric and bispecific antibody-like molecules, a capability that expands therapeutic applications of natural antibodies. This protein engineering platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)).

In some embodiments, the multi-specific binding protein is in the LuZ-Y form, in which a leucine zipper is used to induce heterodimerization of two different HCs. (Wranik, B J. et al., J. Biol. Chem. (2012), 287:43331-9).

In some embodiments, the multi-specific binding protein is in the Cov-X-Body form. In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); 22611-22616).

In some embodiments, the multi-specific binding protein is in an Oasc-Fab heterodimeric form that includes Fab fragment binding to target 1, and scFab binding to target 2 fused to Fc. Heterodimerization is ensured by mutations in the Fc.

In some embodiments, the multi-specific binding protein is in a DuetMab form, which is a heterodimeric construct containing two different Fab fragments binding to antigens 1 and 2, and Fc stabilized by heterodimerization mutations. Fab fragments 1 and 2 contain differential S-S bridges that ensure correct LC and HC pairing.

In some embodiments, the multi-specific binding protein is in a CrossmAb form, which is a heterodimeric construct with two different Fab fragments binding to targets 1 and 2, fused to Fc stabilized by heterodimerization. CL and CH1 domains and VH and VL domains are switched, e.g., CH1 is fused in-frame with VL, while CL is fused in-frame with VH.

In some embodiments, the multi-specific binding protein is in a Fit-Ig form, which is a homodimeric construct where Fab fragment binding to antigen 2 is fused to the N terminus of HC of Fab fragment that binds to antigen 1. The construct contains wild-type Fc.

Individual components of the multi-specific binding proteins are described in more detail below.

### NKG2D-Binding Site

Upon binding to the NKG2D receptor and CD16 receptor on natural killer cells, and a tumor-associated antigen on cancer cells, the multi-specific binding proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys.

Table 1 lists peptide sequences of heavy chain variable domains and light chain variable domains that, in combination, can bind to NKG2D. In some embodiments, the heavy chain variable domain and the light chain variable domain are arranged in Fab format. In some embodiments, the heavy chain variable domain and the light chain variable domain are fused together to from an scFv.

The NKG2D binding sites listed in Table 1 can vary in their binding affinity to NKG2D, nevertheless, they all activate human NK cells.

Unless indicated otherwise, the CDR sequences provided in Table 1 are determined under Kabat.

TABLE 1

| | Heavy chain variable | Light chain variable |
|---|---|---|
| | region amino acid | region amino acid |
| Clones | sequence | sequence |
| ADI- | QVQLQQWGAGLLKPSETLSLTCA | DIQMTQSPSTLSASVGDRVTIT |
| 27705 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYNSYPITFGGGTKVEIK |
| | SS (SEQ ID NO: 138) | (SEQ ID NO: 139) |
| | CDR1 (SEQ ID NO: 140) - | |
| | GSFSGYYWS | |
| | CDR2 (SEQ ID NO: 141) - | |
| | EIDHSGSTNYNPSLKS | |
| | CDR3 (SEQ ID NO: 142) - | |
| | ARARGPWSFDP | |
| ADI- | QVQLQQWGAGLLKPSETLSLTCA | EIVLTQSPGTLSLSPGERATLS |
| 27724 | VYGGSFSGYYWSWIRQPPGKGLE | CRASQSVSSSYLAWYQQKPGQA |
| | WIGEIDHSGSTNYNPSLKSRVTI | PRLLIYGASSRATGIPDRFSGS |
| | SVDTSKNQFSLKLSSVTAADTAV | GSGTDFTLTISRLEPEDFAVYY |
| | YYCARARGPWSFDPWGQGTLVTV | CQQYGSSPITFGGGTKVEIK |
| | SS (SEQ ID NO: 143) | (SEQ ID NO: 144) |
| ADI- | QVQLQQWGAGLLKPSETLSLTCA | DIQMTQSPSTLSASVGDRVTIT |
| 27740 | VYGGSFSGYYWSWIRQPPGKGLE | CRASQSIGSWLAWYQQKPGKAP |
| (A40) | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYHSFYTFGGGTKVEIK |
| | SS (SEQ ID NO: 145) | (SEQ ID NO: 146) |
| ADI- | QVQLQQWGAGLLKPSETLSLTCA | DIQMTQSPSTLSASVGDRVTIT |
| 27741 | VYGGSFSGYYWSWIRQPPGKGLE | CRASQSIGSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQSNSYYTFGGGTKVEIK |
| | SS (SEQ ID NO: 147) | (SEQ ID NO: 148) |
| ADI- | QVQLQQWGAGLLKPSETLSLTCA | DIQMTQSPSTLSASVGDRVTIT |
| 27743 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYNSYPTFGGGTKVEIK |
| | SS (SEQ ID NO: 149) | (SEQ ID NO: 150) |
| ADI- | QVQLQQWGAGLLKPSETLSLTCA | ELQMTQSPSSLSASVGDRVTIT |
| 28153 | VYGGSFSGYYWSWIRQPPGKGLE | CRTSQSISSYLNWYQQKPGQPP |
| | WIGEIDHSGSTNYNPSLKSRVTI | KLLIYWASTRESGVPDRFSGSG |
| | SVDTSKNQFSLKLSSVTAADTAV | SGTDFTLTISSLQPEDSATYYC |
| | YYCARARGPWGFDPWGQGTLVTV | QQSYDIPYTFGQGTKLEIK |
| | SS (SEQ ID NO: 151) | (SEQ ID NO: 152) |
| ADI- | QVQLQQWGAGLLKPSETLSLTCA | DIQMTQSPSTLSASVGDRVTIT |
| 28226 | VYGGSFSGYYWSWIRQPPGKGLE | CRASQSISSWLAWYQQKPGKAP |
| (C26) | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYGSFPITFGGGTKVEIK |
| | SS (SEQ ID NO: 153) | (SEQ ID NO: 154) |
| ADI- | QVQLQQWGAGLLKPSETLSLTCA | DIQMTQSPSTLSASVGDRVTIT |
| 28154 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTDFTLTISSLQPDDFATYYC |
| | YYCARARGPWSFDPWGQGTLVTV | QQSKEVPWTFGQGTKVEIK |
| | SS (SEQ ID NO: 155) | (SEQ ID NO: 156) |
| ADI- | QVQLQQWGAGLLKPSETLSLTCA | DIQMTQSPSTLSASVGDRVTIT |
| 29399 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYNSFPTFGGGTKVEIK |
| | SS (SEQ ID NO: 157) | (SEQ ID NO: 158) |
| ADI- | QVQLQQWGAGLLKPSETLSLTCA | DIQMTQSPSTLSASVGDRVTIT |
| 29401 | VYGGSFSGYYWSWIRQPPGKGLE | CRASQSIGSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYDIYPTFGGGTKVEIK |
| | SS (SEQ ID NO: 159) | (SEQ ID NO: 160) |
| ADI- | QVQLQQWGAGLLKPSETLSLTCA | DIQMTQSPSTLSASVGDRVTIT |
| 29403 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYDSYPTFGGGTKVEIK |
| | SS (SEQ ID NO: 162) | (SEQ ID NO: 163) |
| ADI- | QVQLQQWGAGLLKPSETLSLTCA | DIQMTQSPSTLSASVGDRVTIT |
| 29405 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYGSFPTFGGGTKVEIK |
| | SS (SEQ ID NO: 164) | (SEQ ID NO: 165) |
| ADI- | QVQLQQWGAGLLKPSETLSLTCA | DIQMTQSPSTLSASVGDRVTIT |
| 29407 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYQSFPTFGGGTKVEIK |
| | SS (SEQ ID NO: 166) | (SEQ ID NO: 167) |
| ADI- | QVQLQQWGAGLLKPSETLSLTCA | DIQMTQSPSTLSASVGDRVTIT |
| 29419 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYSSFSTFGGGTKVEIK |
| | SS (SEQ ID NO: 168) | (SEQ ID NO: 169) |
| ADI- | QVQLQQWGAGLLKPSETLSLTCA | DIQMTQSPSTLSASVGDRVTIT |
| 29421 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYESYSTFGGGTKVEIK |
| | SS (SEQ ID NO: 170) | (SEQ ID NO: 171) |
| ADI- | QVQLQQWGAGLLKPSETLSLTCA | DIQMTQSPSTLSASVGDRVTIT |
| 29424 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYDSFITFGGGTKVEIK |
| | SS (SEQ ID NO: 172) | (SEQ ID NO: 173) |
| ADI- | QVQLQQWGAGLLKPSETLSLTCA | DIQMTQSPSTLSASVGDRVTIT |
| 29425 | VYGGSFSGYYWSWIRQPPGKGLE | CRASQSISSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYQSYPTFGGGTKVEIK |
| | SS (SEQ ID NO: 174) | (SEQ ID NO: 175) |
| ADI- | QVQLQQWGAGLLKPSETLSLTCA | DIQMTQSPSTLSASVGDRVTIT |
| 29426 | VYGGSFSGYYWSWIRQPPGKGLE | CRASQSIGSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYHSFPTFGGGTKVEIK |
| | SS (SEQ ID NO: 176) | (SEQ ID NO: 177) |
| ADI- | QVQLQQWGAGLLKPSETLSLTCA | DIQMTQSPSTLSASVGDRVTIT |
| 29429 | VYGGSFSGYYWSWIRQPPGKGLE | CRASQSIGSWLAWYQQKPGKAP |
| | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYELYSYTFGGGTKVEIK |
| | SS (SEQ ID NO: 178) | (SEQ ID NO: 179) |
| ADI- | QVQLQQWGAGLLKPSETLSLTCA | DIQMTQSPSTLSASVGDRVTIT |
| 29447 | VYGGSFSGYYWSWIRQPPGKGLE | CRASQSISSWLAWYQQKPGKAP |
| (F47) | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | QQYDTFITFGGGTKVEIK |
| | SS (SEQ ID NO: 180) | (SEQ ID NO: 181) |
| ADI- | QVQLVQSGAEVKKPGSSVKVSCK | DIVMTQSPDSLAVSLGERATIN |
| 27727 | ASGGTFSSYAISWVRQAPGQGLE | CKSSQSVLYSSNNKNYLAWYQQ |
| | WMGGIIPIFGTANYAQKFQGRVT | KPGQPPKLLIYWASTRESGVPD |
| | ITADESTSTAYMELSSLRSEDTA | RFSGSGSGTDFTLTISSLQAED |
| | VYYCARGDSSIRHAYYYYGMDVW | VAVYYCQQYYSTPITFGGGTKV |
| | GQGTTVTVSS | EIK (SEQ ID NO: 183) |
| | (SEQ ID NO: 182) | |
| | CDR1 (SEQ ID NO: 184) - | CDR1 (SEQ ID NO: 187) - |
| | GTFSSYAIS (non-Kabat) | KSSQSVLYSSNNKNYLA |
| | or SYAIS | CDR2 (SEQ ID NO: 188) - |
| | (SEQ ID NO: 185) | WASTRES |
| | CDR2 (SEQ ID NO: 186) - | CDR3 (SEQ ID NO: 191) - |
| | GIIPIFGTANYAQKFQG | QQYYSTPIT |
| | CDR3 (SEQ ID NO: 189) - | |
| | ARGDSSIRHAYYYYGMDV | |
| | (non-Kabat) or | |
| | GDSSIRHAYYYYGMDV (SEQ | |
| | ID NO: 190) | |
| ADI- | QLQLQESGPGLVKPSETLSLTCT | EIVLTQSPATLSLSPGERATLS |
| 29443 | VSGGSISSSSYYWGWIRQPPGKG | CRASQSVSRYLAWYQQKPGQAP |
| (F43) | LEWIGSIYYSGSTYYNPSLKSRV | RLLIYDASNRATGIPARFSGSG |
| | TISVDTSKNQFSLKLSSVTAADT | SGTDFTLTISSLEPEDFAVYYC |
| | AVYYCARGSDRFHPYFDYWGQGT | QQFDTWPPTFGGGTKVEIK |
| | LVTVSS (SEQ ID NO: 192) | (SEQ ID NO: 161) |
| | CDR1 (SEQ ID NO: 193)- | CDR1 (SEQ ID NO: 198) - |
| | GSISSSSYYWG (non-Kabat) | RASQSVSRYLA |
| | or SSSYYWG (SEQ ID NO: | CDR2 (SEQ ID NO: 199) - |
| | 194) | DASNRAT |
| | CDR2 (SEQ ID NO: 195) - | CDR3 (SEQ ID NO: 200) - |
| | SIYYSGSTYYNPSLKS | QQFDTWPPT |
| | CDR3 (SEQ ID NO: 196) - | |
| | ARGSDRFHPYFDY (non- | |
| | Kabat) or GSDRFHPYFDY | |
| | (SEQ ID NO: 197) | |
| ADI- | QVQLQQWGAGLLKPSETLSLTCA | DIQMTQSPSTLSASVGDRVTIT |
| 29404 | VYGGSFSGYYWSWIRQPPGKGLE | CRASQSISSWLAWYQQKPGKAP |
| (F04) | WIGEIDHSGSTNYNPSLKSRVTI | KLLIYKASSLESGVPSRFSGSG |
| | SVDTSKNQFSLKLSSVTAADTAV | SGTEFTLTISSLQPDDFATYYC |
| | YYCARARGPWSFDPWGQGTLVTV | EQYDSYPTFGGGTKVEIK |
| | SS (SEQ ID NO: 201) | (SEQ ID NO: 202) |
| ADI- | QVQLVQSGAEVKKPGSSVKVSCK | DIVMTQSPDSLAVSLGERATIN |
| 28200 | ASGGTFSSYAISWVRQAPGQGLE | CESSQSLLNSGNQKNYLTWYQQ |
| | WMGGIIPIFGTANYAQKFQGRVT | KPGQPPKPLIYWASTRESGVPD |
| | ITADESTSTAYMELSSLRSEDTA | RFSGSGSGTDFTLTISSLQAED |
| | VYYCARRGRKASGSFYYYYGMDV | VAVYYCQNDYSYPYTFGQGTKL |
| | WGQGTTVTVSS | EIK (SEQ ID NO: 204) |
| | (SEQ ID NO: 203) | CDR1 (SEQ ID NO: 207) - |
| | CDR1 (SEQ ID NO: 184) - | ESSQSLLNSGNQKNYLT |
| | GTFSSYAIS | CDR2 (SEQ ID NO: 188) - |
| | CDR2 (SEQ ID NO: 205) - | WASTRES |
| | GIIPIFGTANYAQKFQG | CDR3 (SEQ ID NO: 208) - |
| | CDR3 (SEQ ID NO: 206) - | QNDYSYPYT |
| | ARRGRKASGSFYYYYGMDV | |
| ADI- | QVQLVQSGAEVKKPGASVKVSCK | EIVMTQSPATLSVSPGERATLS |
| 29379 | ASGYTFTSYYMHWVRQAPGQGLE | CRASQSVSSNLAWYQQKPGQAP |
| (E79) | WMGIINPSGGSTSYAQKFQGRVT | RLLIYGASTRATGIPARFSGSG |
| | MTRDTSTSTVYMELSSLRSEDTA | SGTEFTLTISSLQSEDFAVYYC |
| | VYYCARGAPNYGDTTHDYYYMDV | QQYDDWPFTFGGGTKVEIK |
| | WGKGTTVTVSS | (SEQ ID NO: 210) |
| | (SEQ ID NO: 209) | CDR1 (SEQ ID NO: 216) - |
| | CDR1 (SEQ ID NO: 211) - | RASQSVSSNLA |
| | YTFTSYYMH (non-Kabat) | CDR2 (SEQ ID NO: 217) - |
| | or SYYMH (SEQ ID NO: | GASTRAT |
| | 212) | CDR3 (SEQ ID NO: 218) - |
| | CDR2 (SEQ ID NO: 213) - | QQYDDWPFT |
| | IINPSGGSTSYAQKFQG | |
| | CDR3 (SEQ ID NO: 214) - | |
| | ARGAPNYGDTTHDYYYMDV | |
| | (non-Kabat) or | |
| | GAPNYGDTTHDYYYMDV (SEQ | |
| | ID NO: 215) | |
| ADI- | QVQLVQSGAEVKKPGASVKVSCK | EIVLTQSPGTLSLSPGERATLS |
| 29463 | ASGYTFTGYYMHWVRQAPGQGLE | CRASQSVSSNLAWYQQKPGQAP |
| (F63) | WMGWINPNSGGTNYAQKFQGRVT | RLLIYGASTRATGIPARFSGSG |
| | MTRDTSISTAYMELSRLRSDDTA | SGTEFTLTISSLQSEDFAVYYC |
| | VYYCARDTGEYYDTDDHGMDVWG | QQDDYWPPTFGGGTKVEIK |
| | QGTTVTVSS (SEQ ID NO: | (SEQ ID NO: 220) |
| | 219) | CDR1 (SEQ ID NO: 226) - |
| | CDR1 (SEQ ID NO: 221) - | RASQSVSSNLA |
| | YTFTGYYMH (non-Kabat) | CDR2 (SEQ ID NO: 217) - |
| | or GYYMH (SEQ ID NO: | GASTRAT |
| | 222) | CDR3 (SEQ ID NO: 227) - |
| | CDR2 (SEQ ID NO: 223) - | QQDDYWPPT |
| | WINPNSGGTNYAQKFQG | |
| | CDR3 (SEQ ID NO: 224) - | |
| | ARDTGEYYDTDDHGMDV (non- | |
| | Kabat) or DTGEYYDTDDHGM | |
| | DV (SEQ ID NO: 225) | |
| ADI- | EVQLLESGGGLVQPGGSLRLSCA | DIQMTQSPSSVSASVGDRVTIT |
| 27744 | ASGFTFSSYAMSWVRQAPGKGLE | CRASQGIDSWLAWYQQKPGKAP |
| (A44) | WVSAISGSGGSTYYADSVKGRFT | KLLIYAASSLQSGVPSRFSGSG |
| | ISRDNSKNTLYLQMNSLRAEDTA | SGTDFTLTISSLQPEDFATYYC |
| | VYYCAKDGGYYDSGAGDYWGQGT | QQGVSYPRTFGGGTKVEIK |
| | LVTVSS (SEQ ID NO: 228) | (SEQ ID NO: 229) |
| | CDR1 (SEQ ID NO: 230) - | CDR1 (SEQ ID NO: 235) - |
| | FTFSSYAMS (non-Kabat) | RASQGIDSWLA |
| | or SYAMS (SEQ ID NO: | CDR2 (SEQ ID NO: 236) - |
| | 231) | AASSLOS |
| | CDR2 (SEQ ID NO: 232) - | CDR3 (SEQ ID NO: 237) - |
| | AISGSGGSTYYADSVKG | QQGVSYPRT |
| | CDR3 (SEQ ID NO: 233) - | |
| | AKDGGYYDSGAGDY (non- | |
| | Kabat) or DGGYYDSGAGDY | |
| | (SEQ ID NO: 234) | |
| ADI- | EVQLVESGGGLVKPGGSLRLSCA | DIQMTQSPSSVSASVGDRVTIT |
| 27749 | ASGFTFSSYSMNWVRQAPGKGLE | CRASQGISSWLAWYQQKPGKAP |
| (A49) | WVSSISSSSSYIYYADSVKGRFT | KLLIYAASSLQSGVPSRFSGSG |
| | ISRDNAKNSLYLQMNSLRAEDTA | SGTDFTLTISSLQPEDFATYYC |
| | VYYCARGAPMGAAAGWFDPWGQG | QQGVSFPRTFGGGTKVEIK |
| | TLVTVSS | (SEQ ID NO: 239) |
| | (SEQ ID NO: 238) | CDR1 (SEQ ID NO: 276) - |
| | CDR1 (SEQ ID NO: 240) - | RASQGISSWLA |
| | FTFSSYSMN (non-Kabat) | CDR2 (SEQ ID NO: 236) - |
| | or SYSMN (SEQ ID NO: | AASSLQS |
| | 241) | CDR3 (SEQ ID NO: 245) - |
| | CDR2 (SEQ ID NO: 242) - | QQGVSFPRT |
| | SISSSSSYIYYADSVKG | |
| | CDR3 (SEQ ID NO: 243) - | |
| | ARGAPMGAAAGWFDP (non- | |
| | Kabat) or GAPMGAAAGWFDP | |
| | (SEQ ID NO: 244) | |
| | scFv (VL-VH) with Q44C in VH and G100C | |
| | in VL, linker underlined: | |
| | DIQMTQSPSSVSASVGDRVTITCRASQGISSWLAWYQQKPGKAPKLL | |
| | IYAASSLQSGVPSRFSGSGSGTDFTLTISSLQPEDFATYYCQQGVSF | |
| | PRTFGCGTKVEIKGGGGSGGGGSGGGGSGGGGSEVQLVESGGGLVKP | |
| | GGSLRLSCAASGFTFSSYSMNWVRQAPGKCLEWVSSISSSSSYIYYA | |
| | DSVKGRFTISRDNAKNSLYLQMNSLRAEDTAVYYCARGAPMGAAAGW | |
| | FDPWGQGTLVTVSS (SEQ ID NO: 246) | |
| ADI- | QVQLVQSGAEVKKPGASVKVSCK | EIVLTQSPATLSLSPGERATLS |
| 29378 | ASGYTFTSYYMHWVRQAPGQGLE | CRASQSVSSYLAWYQQKPGQAP |
| (E78) | WMGIINPSGGSTSYAQKFQGRVT | RLLIYDASNRATGIPARFSGSG |
| | MTRDTSTSTVYMELSSLRSEDTA | SGTDFTLTISSLEPEDFAVYYC |
| | VYYCAREGAGFAYGMDYYYMDVW | QQSDNWPFTFGGGTKVEIK |
| | GKGTTVTVSS | (SEQ ID NO: 248) |
| | (SEQ ID NO: 247) | CDR1 (SEQ ID NO: 252) - |
| | CDR1 (SEQ ID NO: 211) - | RASQSVSSYLA |
| | YTFTSYYMH (non-Kabat) | CDR2 (SEQ ID NO: 199) - |
| | or SYYMH (SEQ ID NO: | DASNRAT |
| | 212) | CDR3 (SEQ ID NO: 253) - |
| | CDR2 (SEQ ID NO: 249) - | QQSDNWPFT |
| | IINPSGGSTSYAQKFQG | |
| | CDR3 (SEQ ID NO: 250) - | |
| | AREGAGFAYGMDYYYMDV | |
| | (non-Kabat) or | |
| | EGAGFAYGMDYYYMDV (SEQ | |
| | ID NO: 251) | |
| A49MI | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGKAP |
| | WVSSISSSSSYIYYADSVKGRFT | KLLIYAASSLQSGVPSRFSGSG |
| | ISRDNAKNSLYLQMNSLRAEDTA | SGTDFTLTISSLQPEDFATYYC |
| | VYYCARGAPIGAAAGWFDPWGQG | QQGVSFPRTFGGGTKVEIK |
| | TLVTVSS (SEQ ID NO: | (SEQ ID NO: 239) |
| | 254) | CDR1 (SEQ ID NO: 276) - |
| | CDR1: FTFSSYSMN (SEQ ID | RASQGISSWLA |
| | NO: 240) (non-Kabat) or | CDR2 (SEQ ID NO: 236) - |
| | SYSMN (SEQ ID NO: 241) | AASSLOS |
| | CDR2: SISSSSSYIYYADSVKG | CDR3 (SEQ ID NO: 245) - |
| | (SEQ ID NO: 242) | QQGVSFPRT |
| | CDR3: (non-Kabat) | |
| | ARGAPIGAAAGWFDP (SEQ ID | |
| | NO: 255) or GAPIGAAAGWF | |
| | DP (SEQ ID NO: 256) | |
| A49MQ | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGKAP |
| | WVSSISSSSSYIYYADSVKGRFT | KLLIYAASSLQSGVPSRFSGSG |
| | ISRDNAKNSLYLQMNSLRAEDTA | SGTDFTLTISSLQPEDFATYYC |
| | VYYCARGAPQGAAAGWFDPWGQG | QQGVSFPRTFGGGTKVEIK |
| | TLVTVSS (SEQ ID NO: | (SEQ ID NO: 239) |
| | 257) | CDR1 (SEQ ID NO: 276) - |
| | CDR1: FTFSSYSMN (SEQ ID | RASQGISSWLA |
| | NO: 240) (non-Kabat) or | CDR2 (SEQ ID NO: 236) - |
| | SYSMN (SEQ ID NO: 241) | AASSLQS |
| | CDR2: SISSSSSYIYYADSVKG | CDR3 (SEQ ID NO: 245) - |
| | (SEQ ID NO: 242) | QQGVSFPRT |
| | CDR3 (non-Kabat) (SEQ | |
| | ID NO: 258) - ARGAPQGAA | |
| | AGWFDP or CDR3 (SEQ ID | |
| | NO: 259) - GAPQGAAAGWFD | |
| | P | |
| A49ML | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGKAP |
| | WVSSISSSSSYIYYADSVKGRFT | KLLIYAASSLQSGVPSRFSGSG |
| | ISRDNAKNSLYLQMNSLRAEDTA | SGTDFTLTISSLQPEDFATYYC |
| | VYYCARGAPLGAAAGWFDPWGQG | QQGVSFPRTFGGGTKVEIK |
| | TLVTVSS (SEQ ID NO: | (SEQ ID NO: 239) |
| | 260) | CDR1 (SEQ ID NO: 276) - |
| | CDR1: FTFSSYSMN (SEQ ID | RASQGISSWLA |
| | NO: 240) (non-Kabat) or | CDR2 (SEQ ID NO: 236) - |
| | SYSMN (SEQ ID NO: 241) | AASSLQS |
| | CDR2: SISSSSSYIYYADSVKG | CDR3 (SEQ ID NO: 245) - |
| | (SEQ ID NO: 242) | QQGVSFPRT |
| | CDR3 (non-Kabat) (SEQ | |
| | ID NO: 261) - ARGAPLGAA | |
| | AGWFDP or CDR3 (SEQ ID | |
| | NO: 262) - GAPLGAAAGWFD | |
| | P | |
| A49MF | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGKAP |
| | WVSSISSSSSYIYYADSVKGRFT | KLLIYAASSLQSGVPSRFSGSG |
| | ISRDNAKNSLYLQMNSLRAEDTA | SGTDFTLTISSLQPEDFATYYC |
| | VYYCARGAPFGAAAGWFDPWGQG | QQGVSFPRTFGGGTKVEIK |
| | TLVTVSS (SEQ ID NO: | (SEQ ID NO: 239) |
| | 263) | CDR1 (SEQ ID NO: 276) - |
| | CDR1: FTFSSYSMN (SEQ ID | RASQGISSWLA |
| | NO: 240) (non-Kabat) or | CDR2 (SEQ ID NO: 236) - |
| | SYSMN (SEQ ID NO: 241) | AASSLQS |
| | CDR2: SISSSSSYIYYADSVKG | CDR3 (SEQ ID NO: 245) - |
| | (SEQ ID NO: 242) | QQGVSFPRT |
| | CDR3 (non-Kabat) (SEQ | |
| | ID NO: 264) - ARGAPFGAA | |
| | AGWFDP or CDR3 (SEQ ID | |
| | NO: 265) - GAPFGAAAGWFD | |
| | P | |
| A49MV | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGKAP |
| | WVSSISSSSSYIYYADSVKGRFT | KLLIYAASSLQSGVPSRFSGSG |
| | ISRDNAKNSLYLQMNSLRAEDTA | SGTDFTLTISSLQPEDFATYYC |
| | VYYCARGAPVGAAAGWFDPWGQG | QQGVSFPRTFGGGTKVEIK |
| | TLVTVSS (SEQ ID NO: | (SEQ ID NO: 239) |
| | 266) | CDR1 (SEQ ID NO: 276) - |
| | CDR1: FTFSSYSMN (SEQ ID | RASQGISSWLA |
| | NO: 240) (non-Kabat) or | CDR2 (SEQ ID NO: 236) - |
| | SYSMN (SEQ ID NO: 241) | AASSLQS |
| | CDR2: SISSSSSYIYYADSVKG | CDR3 (SEQ ID NO: 245) - |
| | (SEQ ID NO: 242) | QQGVSFPRT |
| | CDR3 (non-Kabat) (SEQ | |
| | ID NO: 267) - ARGAPVGAA | |
| | AGWFDP or CDR3 (SEQ ID | |
| | NO: 268) - GAPVGAAAGWFD | |
| | P | |
| A49- | EVQLVESGGGLVKPGGSLRLSCA | DIQMTQSPSSVSASVGDRVTIT |
| consensus | ASGFTFSSYSMNWVRQAPGKGLE | CRASQGISSWLAWYQQKPGKAP |
| | WVSSISSSSSYIYYADSVKGRFT | KLLIYAASSLQSGVPSRFSGSG |
| | ISRDNAKNSLYLQMNSLRAEDTA | SGTDFTLTISSLQPEDFATYYC |
| | VYYCARGAPXGAAAGWFDPWGQG | QQGVSFPRTFGGGTKVEIK |
| | TLVTVSS, wherein X is | (SEQ ID NO: 239) |
| | M, L, I, V, Q, or F | CDR1 (SEQ ID NO: 276) - |
| | (SEQ ID NO: 269) | RASQGISSWLA |
| | CDR1: FTFSSYSMN (SEQ ID | CDR2 (SEQ ID NO: 236) - |
| | NO: 240) (non-Kabat) or | AASSLOS |
| | SYSMN (SEQ ID NO: 241 ) | CDR3 (SEQ ID NO: 245) - |
| | CDR2: SISSSSSYIYYADSVKG | QQGVSFPRT |
| | (SEQ ID NO: 242) | |
| | CDR3 (non-Kabat) (SEQ | |
| | ID NO: 270) - ARGAPXGAA | |
| | AGWFDP or CDR3 (SEQ ID | |
| | NO: 271) - GAPXGAAAGWFD | |
| | P, wherein X is M, L, | |
| | I, V, Q, or F | |
| NKG2D | QVQLVESGGGLVKPGGSLRLSCA | QSALTQPASVSGSPGQSITISC |
| binder in | ASGFTFSSYGMHWVRQAPGKGLE | SGSSSNIGNNAVNWYQQLPGKA |
| U.S. Pat. | WVAFIRYDGSNKYYADSVKGRFT | PKLLIYYDDLLPSGVSDRFSGS |
| No. | ISRDNSKNTLYLQMNSLRAEDTA | KSGTSAFLAISGLQSEDEADYY |
| 9,273,136 | VYYCAKDRGLGDGTYFDYWGQGT | CAAWDDSLNGPVFGGGTKLTVL |
| | TVTVSS (SEQ ID NO: 272) | (SEQ ID NO: 273) |
| NKG2D | QVHLQESGPGLVKPSETLSLTCT | EIVLTQSPGTLSLSPGERATLS |
| binder in | VSDDSISSYYWSWIRQPPGKGLE | CRASQSVSSSYLAWYQQKPGQA |
| U.S. Pat. | WIGHISYSGSANYNPSLKSRVTI | PRLLIYGASSRATGIPDRFSGS |
| No. | SVDTSKNQFSLKLSSVTAADTAV | GSGTDFTLTISRLEPEDFAVYY |
| 7,879,985 | YYCANWDDAFNIWGQGTMVTVSS | CQQYGSSPWTFGQGTKVEIK |
| | (SEQ ID NO: 274) | (SEQ ID NO: 275) |

In certain embodiments, the first antigen-binding site that binds NKG2D (e.g., human NKG2D) comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VH of the same antibody disclosed in Table 1. In certain embodiments, the first antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antibody discloses in Table 1. In certain embodiments, the first antigen-binding site comprises the heavy chain CDR1, CDR2, and CDR3 and the light chain CDR1, CDR2, and CDR3 of an antibody disclosed in Table 1.

In certain embodiments, the first antigen-binding site that binds to NKG2D comprises a heavy chain variable domain related to SEQ ID NO:138, such as by having an amino acid sequence at least 90% (e.g., at least 90%, at least 91%, at least 92%, at least 93%, at least 94%, at least 95%, at least 96%, at least 97%, at least 98%, at least 99%, or 100%) identical to SEQ ID NO:138, and/or incorporating amino acid sequences identical to the CDR1 (SEQ ID NO:140), CDR2 (SEQ ID NO:141), and CDR3 (SEQ ID NO:142) sequences of SEQ ID NO:138. The heavy chain variable domain related to SEQ ID NO:138 can be coupled with a variety of light chain variable domains to form an NKG2D binding site. For example, the first antigen-binding site that incorporates a heavy chain variable domain related to SEQ ID NO:138 can further incorporate a light chain variable domain selected from any one of the sequences related to SEQ ID NOs: 139, 144, 146, 148, 150, 154, 156, 158, 160, 163, 165, 167, 169, 171, 173, 175, 177, 179, and 181. For example, the first antigen-binding site incorporates a heavy chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to SEQ ID NO:138 and a light chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to any one of the sequences selected from SEQ ID NOs: 139, 144, 146, 148, 150, 154, 156, 158, 160, 163, 165, 167, 169, 171, 173, 175, 177, 179, and 181.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:182, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:183. In certain embodiments, the VH comprises CDR1, CDR2, and CDR3 comprising the amino acid sequences of SEQ ID NOs: 184 or 185, 186, and 189 or 190, respectively. In certain embodiments, the VL comprises CDR1, CDR2, and CDR3 comprising the amino acid sequences of SEQ ID NOs: 187, 188, and 191, respectively. In certain embodiments, the first antigen-binding site comprises (a) a VH that comprises CDR1, CDR2, and CDR3 comprising the amino acid sequences of SEQ ID NOs: 184 or 185, 186, and 189 or 190, respectively; and (b) a VL that comprises CDR1, CDR2, and CDR3 comprising the amino acid sequences of SEQ ID NOs: 187, 188, and 191, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:192, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:161. In certain embodiments, the VH comprises CDR1, CDR2, and CDR3 comprising the amino acid sequences of SEQ ID NOs: 193 or 194, 195, and 196 or 197, respectively. In certain embodiments, the VL comprises CDR1, CDR2, and CDR3 comprising the amino acid sequences of SEQ ID NOs: 198, 199, and 200, respectively. In certain embodiments, the first antigen-binding site comprises (a) a VH that comprises CDR1, CDR2, and CDR3 comprising the amino acid sequences of SEQ ID NOs: 193 or 194, 195, and 196 or 197, respectively; and (b) a VL that comprises CDR1, CDR2, and CDR3 comprising the amino acid sequences of SEQ ID NOs: 198, 199, and 200, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:201, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:202.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:203, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:204. In certain embodiments, the VH comprises CDR1, CDR2, and CDR3 comprising the amino acid sequences of SEQ ID NOs: 184, 205, and 206, respectively. In certain embodiments, the VL comprises CDR1, CDR2, and CDR3 comprising the amino acid sequences of SEQ ID NOs: 207, 188, and 208, respectively. In certain embodiments, the first antigen-binding site comprises (a) a VH that comprises CDR1, CDR2, and CDR3 comprising the amino acid sequences of SEQ ID NOs: 184, 205, and 206, respectively; and (b) a VL that comprises CDR1, CDR2, and CDR3 comprising the amino acid sequences of SEQ ID NOs: 207, 188, and 208, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:209, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:210. In certain embodiments, the VH comprises CDR1, CDR2, and CDR3 comprising the amino acid sequences of SEQ ID NOs: 211 or 212, 213, and 214 or 215, respectively. In certain embodiments, the VL comprises CDR1, CDR2, and CDR3 comprising the amino acid sequences of SEQ ID NOs: 216, 217, and 218, respectively. In certain embodiments, the first antigen-binding site comprises (a) a VH that comprises CDR1, CDR2, and CDR3 comprising the amino acid sequences of SEQ ID NOs: 211 or 212, 213, and 214 or 215, respectively; and (b) a VL that comprises CDR1, CDR2, and CDR3 comprising the amino acid sequences of SEQ ID NOs: 216, 217, and 218, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:219, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:220. In certain embodiments, the VH comprises CDR1, CDR2, and CDR3 comprising the amino acid sequences of SEQ ID NOs: 221 or 222, 223, and 224 or 225, respectively. In certain embodiments, the VL comprises CDR1, CDR2, and CDR3 comprising the amino acid sequences of SEQ ID NOs: 226, 217, and 227, respectively. In certain embodiments, the first antigen-binding site comprises (a) a VH that comprises CDR1, CDR2, and CDR3 comprising the amino acid sequences of SEQ ID NOs: 221 or 222, 223, and 224 or 225, respectively; and (b) a VL that comprises CDR1, CDR2, and CDR3 comprising the amino acid sequences of SEQ ID NOs: 226, 217, and 227, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:247, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:248. In certain embodiments, the VH comprises CDR1, CDR2, and CDR3 comprising the amino acid sequences of SEQ ID NOs: 211 or 212, 249, and 250 or 251, respectively. In certain embodiments, the VL comprises CDR1, CDR2, and CDR3 comprising the amino acid sequences of SEQ ID NOs: 252, 199, and 253, respectively. In certain embodiments, the first antigen-binding site comprises (a) a VH that comprises CDR1, CDR2, and CDR3 comprising the amino acid sequences of SEQ ID NOs: 211 or 212, 249, and 250 or 251, respectively; and (b) a VL that comprises CDR1, CDR2, and CDR3 comprising the amino acid sequences of SEQ ID NOs: 252, 199, and 253, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:228, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:229. In certain embodiments, the VH comprises CDR1, CDR2, and CDR3 comprising the amino acid sequences of SEQ ID NOs: 230 or 231, 232, and 233 or 234, respectively. In certain embodiments, the VL comprises CDR1, CDR2, and CDR3 comprising the amino acid sequences of SEQ ID NOs: 235, 236, and 237, respectively. In certain embodiments, the first antigen-binding site comprises (a) a VH that comprises CDR1, CDR2, and CDR3 comprising the amino acid sequences of SEQ ID NOs: 230 or 231, 232, and 233 or 234, respectively; and (b) a VL that comprises CDR1, CDR2, and CDR3 comprising the amino acid sequences of SEQ ID NOs: 235, 236, and 237, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:238, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:239. In certain embodiments, the VH comprises CDR1, CDR2, and CDR3 comprising the amino acid sequences of SEQ ID NOs: 240 or 241, 242, and 243 or 244, respectively. In certain embodiments, the VL comprises CDR1, CDR2, and CDR3 comprising the amino acid sequences of SEQ ID NOs: 276, 236, and 245, respectively. In certain embodiments, the first antigen-binding site comprises (a) a VH that comprises CDR1, CDR2, and CDR3 comprising the amino acid sequences of SEQ ID NOs: 240 or 241, 242, and 243 or 244 respectively; and (b) a VL that comprises CDR1, CDR2, and CDR3 comprising the amino acid sequences of SEQ ID NOs: 276, 236, and 245, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:254, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:239. In certain embodiments, the VH comprises CDR1, CDR2, and CDR3 comprising the amino acid sequences of SEQ ID NOs: 240 or 241, 242, and 255 or 256, respectively. In certain embodiments, the VL comprises CDR1, CDR2, and CDR3 comprising the amino acid sequences of SEQ ID NOs: 276, 236, and 245, respectively. In certain embodiments, the first antigen-binding site comprises (a) a VH that comprises CDR1, CDR2, and CDR3 comprising the amino acid sequences of SEQ ID NOs: 240 or 241, 242, and 255 or 256, respectively; and (b) a VL that comprises CDR1, CDR2, and CDR3 comprising the amino acid sequences of SEQ ID NOs: 276, 236, and 245, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:257, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:239. In certain embodiments, the VH comprises CDR1, CDR2, and CDR3 comprising the amino acid sequences of SEQ ID NOs: 240 or 241, 242, and 258 or 259, respectively. In certain embodiments, the VL comprises CDR1, CDR2, and CDR3 comprising the amino acid sequences of SEQ ID NOs: 276, 236, and 245, respectively. In certain embodiments, the first antigen-binding site comprises (a) a VH that comprises CDR1, CDR2, and CDR3 comprising the amino acid sequences of SEQ ID NOs: 240 or 241, 242, and 258 or 259, respectively; and (b) a VL that comprises CDR1, CDR2, and CDR3 comprising the amino acid sequences of SEQ ID NOs: 276, 236, and 245, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:260, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:239. In certain embodiments, the VH comprises CDR1, CDR2, and CDR3 comprising the amino acid sequences of SEQ ID NOs: 240 or 241, 242, and 261 or 262, respectively. In certain embodiments, the VL comprises CDR1, CDR2, and CDR3 comprising the amino acid sequences of SEQ ID NOs: 276, 236, and 245, respectively. In certain embodiments, the first antigen-binding site comprises (a) a VH that comprises CDR1, CDR2, and CDR3 comprising the amino acid sequences of SEQ ID NOs: 240 or 241, 242, and 261 or 262, respectively; and (b) a VL that comprises CDR1, CDR2, and CDR3 comprising the amino acid sequences of SEQ ID NOs: 276, 236, and 245, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:263, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:239. In certain embodiments, the VH comprises CDR1, CDR2, and CDR3 comprising the amino acid sequences of SEQ ID NOs: 240 or 241, 242, and 264 or 265, respectively. In certain embodiments, the VL comprises CDR1, CDR2, and CDR3 comprising the amino acid sequences of SEQ ID NOs: 276, 236, and 245, respectively. In certain embodiments, the first antigen-binding site comprises (a) a VH that comprises CDR1, CDR2, and CDR3 comprising the amino acid sequences of SEQ ID NOs: 240 or 241, 242, and 264 or 265, respectively; and (b) a VL that comprises CDR1, CDR2, and CDR3 comprising the amino acid sequences of SEQ ID NOs: 276, 236, and 245, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:266, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:239. In certain embodiments, the VH comprises CDR1, CDR2, and CDR3 comprising the amino acid sequences of SEQ ID NOs: 240 or 241, 242, and 267 or 268, respectively. In certain embodiments, the VL comprises CDR1, CDR2, and CDR3 comprising the amino acid sequences of SEQ ID NOs: 276, 236, and 245, respectively. In certain embodiments, the first antigen-binding site comprises (a) a VH that comprises CDR1, CDR2, and CDR3 comprising the amino acid sequences of SEQ ID NOs: 240 or 241, 242, and 267 or 268, respectively; and (b) a VL that comprises CDR1, CDR2, and CDR3 comprising the amino acid sequences of SEQ ID NOs: 276, 236, and 245, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:269, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:239. In certain embodiments, the VH comprises CDR1, CDR2, and CDR3 comprising the amino acid sequences of SEQ ID NOs: 240 or 241, 242, and 270 or 271, respectively. In certain embodiments, the VL comprises CDR1, CDR2, and CDR3 comprising the amino acid sequences of SEQ ID NOs: 276, 236, and 245, respectively. In certain embodiments, the first antigen-binding site comprises (a) a VH that comprises CDR1, CDR2, and CDR3 comprising the amino acid sequences of SEQ ID NOs: 240 or 241, 242, and 270 or 271, respectively; and (b) a VL that comprises CDR1, CDR2, and CDR3 comprising the amino acid sequences of SEQ ID NOs: 276, 236, and 245, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:272, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:273.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:274, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:275.

The multi-specific binding proteins can bind to NKG2D-expressing cells, which include but are not limited to NK cells, γδ T cells and CD8⁺αβ T cells. Upon NKG2D binding, the multi-specific binding proteins may block natural ligands, such as ULBP6 and MICA, from binding to NKG2D and activating NK cells.

The multi-specific binding proteins binds to cells expressing CD16, an Fc receptor on the surface of leukocytes including natural killer cells, macrophages, neutrophils, eosinophils, mast cells, and follicular dendritic cells. A protein of the present disclosure binds to NKG2D with an affinity of K_{D} of 2 nM to 120 nM, e.g., 2 nM to 110 nM, 2 nM to 100 nM, 2 nM to 90 nM, 2 nM to 80 nM, 2 nM to 70 nM, 2 nM to 60 nM, 2 nM to 50 nM, 2 nM to 40 nM, 2 nM to 30 nM, 2 nM to 20 nM, 2 nM to 10 nM, about 15 nM, about 14 nM, about 13 nM, about 12 nM, about 11 nM, about 10 nM, about 9 nM, about 8 nM, about 7 nM, about 6 nM, about 5 nM, about 4.5 nM, about 4 nM, about 3.5 nM, about 3 nM, about 2.5 nM, about 2 nM, about 1.5 nM, about 1 nM, between about 0.5 nM to about 1 nM, about 1 nM to about 2 nM, about 2 nM to 3 nM, about 3 nM to 4 nM, about 4 nM to about 5 nM, about 5 nM to about 6 nM, about 6 nM to about 7 nM, about 7 nM to about 8 nM, about 8 nM to about 9 nM, about 9 nM to about 10 nM, about 1 nM to about 10 nM, about 2 nM to about 10 nM, about 3 nM to about 10 nM, about 4 nM to about 10 nM, about 5 nM to about 10 nM, about 6 nM to about 10 nM, about 7 nM to about 10 nM, or about 8 nM to about 10 nM. In some embodiments, NKG2D-binding sites bind to NKG2D with a K_{D} of 10 to 62 nM.

### FLT3-Binding Site

The FLT3-binding site of the multi-specific binding protein disclosed herein comprises a heavy chain variable domain and a light chain variable domain. Table 2 lists some exemplary sequences of heavy chain variable domains and light chain variable domains that, in combination, can bind to FLT3. CDR sequences are identified under Chothia numbering.

TABLE 2
Sequences of Exemplary Antigen-Binding
Sites that Bind FLT3

| Clone | VH | VL |
|---|---|---|
| 12H10.G7 | EVQLQESGPELVKPGASVKMS | NIVLTQSPASLAVSLGQRATISC |
| | CKASGYTFTRYVMHWVKQRPG | RASESVDTYGSSFVHWYQQKPGQ |
| | QGLEWIGFINPYNDDTKYNEK | PPKLLIYLASNLESGVPARFSGS |
| | FKGKATLTSDKSSSTAYMELS | GSRSDFTLTIDPVEADDAATYYC |
| | SLTSEDSAVYHCARWRQLGSL | QQNNEEPWTFGGGTKLEIK |
| | DSWGQGTTLTVSS | [SEQ ID NO: 2] |
| | [SEQ ID NO: 1] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| Humanized | QVQLVQSGAEVKKPGASVKVS | DIVMTQSPASLAVSLGERATINC |
| 12H10.G7 | CKASGYTFTRYVMHWVRQAPG | RASESVDTYGSSFVHWYQQKPGQ |
| GB87/GB95 | QRLEWMGFINPYNDDTKYNEK | PPKLLIYLASNLESGVPDRFSGS |
| (back | FKGRVTITSDTSASTAYMELS | GSRTDFTLTISSLQAEDAATYYC |
| mutations in | SLRSEDTAVYHCARWRQLGSL | QQNNEEPWTFGGGTKVEIK |
| VH and VL | DSWGQGTTVTVSS [SEQ ID | [SEQ ID NO: 10] |
| underlined) | NO: 9] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | GB87 (VH-VL): | |
| humanized | QVQLVQSGAEVKKPGASVKVSCKASGYTFTRYVMHWVRQAPGQ | |
| 12H10.G7 | CLEWMGFINPYNDDTKYNEKFKGRVTITSDTSASTAYMELSSL | |
| GB87/GB95 | RSEDTAVYHCARWRQLGSLDSWGQGTTVTVSSGGGGSGGGGSG | |
| | GGGSGGGGSDIVMTQSPASLAVSLGERATINCRASESVDTYGS | |
| | SFVHWYQQKPGQPPKLLIYLASNLESGVPDRFSGSGSRTDFTL | |
| | TISSLQAEDAATYYCQQNNEEPWTFGCGTKVEIK | |
| | [SEQ ID NO: 3] | |
| | GB95 (VL-VH): | |
| | DIVMTQSPASLAVSLGERATINCRASESVDTYGSSFVHWYQQK | |
| | PGQPPKLLIYLASNLESGVPDRFSGSGSRTDFTLTISSLQAED | |
| | AATYYCQQNNEEPWTFGCGTKVEIKGGGGGGGGSGGGGSGGGG | |
| | SQVQLVQSGAEVKKPGASVKVSCKASGYTFTRYVMHWVRQAPG | |
| | QCLEWMGFINPYNDDTKYNEKFKGRVTITSDTSASTAYMELSS | |
| | LRSEDTAVYHCARWRQLGSLDSWGQGTTVTVSS | |
| | [SEQ ID NO: 12] | |
| Humanized | QVQLVQSGAEVKKPGASVKVS | DIVMTQSPASLAVSLGERATINC |
| 12H10.G7 | CKASGYTFTRYVMHWVRQAPG | RASESVDTYGSSFVHWYQQKPGQ |
| GB88/GB96 | QRLEWMGFINPYNDDTKYNEK | PPKLLIYLASNLESGVPDRFSGS |
| (back | FKGRVTITRDTSASTAYMELS | GSRTDFTLTISSLQAEDAATYYC |
| mutations in | SLRSEDTAVYHCARWRQLGSL | QQNNEEPWTFGGGTKVEIK |
| VH and VL | DSWGQGTTVTVSS | [SEQ ID NO: 10] |
| underlined) | [SEQ ID NO: 13] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | GB88 (VH-VL): | |
| humanized | QVQLVQSGAEVKKPGASVKVSCKASGYTFTRYVMHWVRQAPGQ | |
| 12H10.G7 | CLEWMGFINPYNDDTKYNEKFKGRVTITRDTSASTAYMELSSL | |
| GB88/GB96 | RSEDTAVYHCARWRQLGSLDSWGQGTTVTVSSGGGGSGGGGSG | |
| | GGGSGGGGSDIVMTQSPASLAVSLGERATINCRASESVDTYGS | |
| | SFVHWYQQKPGQPPKLLIYLASNLESGVPDRFSGSGSRTDFTL | |
| | TISSLQAEDAATYYCQQNNEEPWTFGCGTKVEIK [SEQ ID | |
| | NO: 15] | |
| | GB96 (VL-VH): | |
| | DIVMTQSPASLAVSLGERATINCRASESVDTYGSSFVHWYQQK | |
| | PGQPPKLLIYLASNLESGVPDRFSGSGSRTDFTLTISSLQAED | |
| | AATYYCQQNNEEPWTFGCGTKVEIKGGGGSGGGGSGGGGSGGG | |
| | GSQVQLVQSGAEVKKPGASVKVSCKASGYTFTRYVMHWVRQAP | |
| | GQCLEWMGFINPYNDDTKYNEKFKGRVTITRDTSASTAYMELS | |
| | SLRSEDTAVYHCARWRQLGSLDSWGQGTTVTVSS [SEQ ID | |
| | NO: 16] | |
| Humanized | QVQLVQSGAEVKKPGASVKVS | DIVMTQSPASLAVSLGERATINC |
| 12H10.G7 | CKASGYTFTRYVMHWVRQAPG | RASESVDTYGSSFVHWYQQKPGQ |
| GB89/GB97 | QRLEWMGFINPYNDDTKYNEK | PPKLLIYLASNLESGVPDRFSGS |
| (back | FKGRVTITSDTSASTAYMELS | GSRTDFTLTISSLQAEDAATYYC |
| mutations in | SLRSEDTAVYYCARWRQLGSL | QQNNEEPWTFGGGTKVEIK |
| VH and VL | DSWGQGTTVTVSS | [SEQ ID NO: 10] |
| underlined) | [SEQ ID NO: 17] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | GB89 (VH-VL): | |
| humanized | QVQLVQSGAEVKKPGASVKVSCKASGYTFTRYVMHWVRQAPGQ | |
| 12H10.G7 | CLEWMGFINPYNDDTKYNEKFKGRVTITSDTSASTAYMELSSL | |
| GB89/GB97 | RSEDTAVYYCARWRQLGSLDSWGQGTTVTVSSGGGGSGGGGSG | |
| | GGGSGGGGSDIVMTQSPASLAVSLGERATINCRASESVDTYGS | |
| | SFVHWYQQKPGQPPKLLIYLASNLESGVPDRFSGSGSRTDFTL | |
| | TISSLQAEDAATYYCQQNNEEPWTFGCGTKVEIK [SEQ ID | |
| | NO: 19] | |
| | GB97 (VL-VH): | |
| | DIVMTQSPASLAVSLGERATINCRASESVDTYGSSFVHWYQQK | |
| | PGQPPKLLIYLASNLESGVPDRFSGSGSRTDFTLTISSLQAED | |
| | AATYYCQQNNEEPWTFGCGTKVEIKGGGGSGGGGSGGGGSGGG | |
| | GSQVQLVQSGAEVKKPGASVKVSCKASGYTFTRYVMHWVRQAP | |
| | GQCLEWMGFINPYNDDTKYNEKFKGRVTITSDTSASTAYMELS | |
| | SLRSEDTAVYYCARWRQLGSLDSWGQGTTVTVSS [SEQ ID | |
| | NO: 20] | |
| Humanized | QVQLVQSGAEVKKPGASVKVS | DIVMTQSPDSLAVSLGERATINC |
| 12H10.G7 | CKASGYTFTRYVMHWVRQAPG | RASESVDTYGSSFVHWYQQKPGQ |
| GB90/GB98 | QRLEWMGFINPYNDDTKYNEK | PPKLLIYLASNLESGVPDRFSGS |
| (back | FKGRVTITSDTSASTAYMELS | GSRTDFTLTISSLQAEDAATYYC |
| mutations in | SLRSEDTAVYHCARWRQLGSL | QQNNEEPWTFGGGTKVEIK |
| VH and VL | DSWGQGTTVTVSS [SEQ ID | [SEQ ID NO: 22] |
| underlined) | NO: 9] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | GB90 (VH-VL): | |
| humanized | QVQLVQSGAEVKKPGASVKVSCKASGYTFTRYVMHWVRQAPGQ | |
| 12H10.G7 | CLEWMGFINPYNDDTKYNEKFKGRVTITSDTSASTAYMELSSL | |
| GB90/GB98 | RSEDTAVYHCARWRQLGSLDSWGQGTTVTVSSGGGGSGGGGSG | |
| | GGGSGGGGSDIVMTQSPDSLAVSLGERATINCRASESVDTYGS | |
| | SFVHWYQQKPGQPPKLLIYLASNLESGVPDRFSGSGSRTDFTL | |
| | TISSLQAEDAATYYCQQNNEEPWTFGCGTKVEIK [SEQ ID | |
| | NO: 23] | |
| | GB98 (VL-VH): | |
| | DIVMTQSPDSLAVSLGERATINCRASESVDTYGSSFVHWYQQK | |
| | PGQPPKLLIYLASNLESGVPDRFSGSGSRTDFTLTISSLQAED | |
| | AATYYCQQNNEEPWTFGCGTKVEIKGGGGSGGGGSGGGGSGGG | |
| | GSQVQLVQSGAEVKKPGASVKVSCKASGYTFTRYVMHWVRQAP | |
| | GQCLEWMGFINPYNDDTKYNEKFKGRVTITSDTSASTAYMELS | |
| | SLRSEDTAVYHCARWRQLGSLDSWGQGTTVTVSS [SEQ ID | |
| | NO: 24] | |
| Humanized | QVQLVQSGAEVKKPGASVKVS | DIVMTQSPASLAVSLGERATINC |
| 12H10.G7 | CKASGYTFTRYVMHWVRQAPG | RASESVDTYGSSFVHWYQQKPGQ |
| GB91/GB99 | QRLEWMGFINPYNDDTKYNEK | PPKLLIYLASNLESGVPDRFSGS |
| (back | FKGRVTITSDTSASTAYMELS | GSGTDFTLTISSLQAEDAATYYC |
| mutations in | SLRSEDTAVYHCARWRQLGSL | QQNNEEPWTFGGGTKVEIK |
| VH and VL | DSWGQGTTVTVSS | [SEQ ID NO: 26] |
| underlined) | [SEQ ID NO: 9] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | GB91 (VH-VL): | |
| humanized | QVQLVQSGAEVKKPGASVKVSCKASGYTFTRYVMHWVRQAPGQ | |
| 12H10.G7 | CLEWMGFINPYNDDTKYNEKFKGRVTITSDTSASTAYMELSSL | |
| GB91/GB99 | RSEDTAVYHCARWRQLGSLDSWGQGTTVTVSSGGGGSGGGGSG | |
| | GGGSGGGGSDIVMTQSPASLAVSLGERATINCRASESVDTYGS | |
| | SFVHWYQQKPGQPPKLLIYLASNLESGVPDRFSGSGSGTDFTL | |
| | TISSLQAEDAATYYCQQNNEEPWTFGCGTKVEIK [SEQ ID | |
| | NO: 27] | |
| | GB99 (VL-VH): | |
| | DIVMTQSPASLAVSLGERATINCRASESVDTYGSSFVHWYQQK | |
| | PGQPPKLLIYLASNLESGVPDRFSGSGSGTDFTLTISSLQAED | |
| | AATYYCQQNNEEPWTFGCGTKVEIKGGGGSGGGGSGGGGSGGG | |
| | GSQVQLVQSGAEVKKPGASVKVSCKASGYTFTRYVMHWVRQAP | |
| | GQCLEWMGFINPYNDDTKYNEKFKGRVTITSDTSASTAYMELS | |
| | SLRSEDTAVYHCARWRQLGSLDSWGQGTTVTVSS [SEQ ID | |
| | NO: 28] | |
| Humanized | QVQLVQSGAEVKKPGASVKVS | DIVMTQSPASLAVSLGERATINC |
| 12H10.G7 | CKASGYTFTRYVMHWVRQAPG | RASESVDTYGSSFVHWYQQKPGQ |
| GB92/GB100 | QRLEWMGFINPYNDDTKYNEK | PPKLLIYLASNLESGVPDRFSGS |
| (back | FKGRVTITSDTSASTAYMELS | GSRTDFTLTISSLQAEDVATYYC |
| mutations in | SLRSEDTAVYHCARWRQLGSL | QQNNEEPWTFGGGTKVEIK |
| VH and VL | DSWGQGTTVTVSS | [SEQ ID NO: 30] |
| underlined) | [SEQ ID NO: 9] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | GB92 (VH-VL): | |
| humanized | QVQLVQSGAEVKKPGASVKVSCKASGYTFTRYVMHWVRQAPGQ | |
| 12H10.G7 | CLEWMGFINPYNDDTKYNEKFKGRVTITSDTSASTAYMELSSL | |
| GB92/GB100 | RSEDTAVYHCARWRQLGSLDSWGQGTTVTVSSGGGGGGGGSGG | |
| | GGSGGGGSDIVMTQSPASLAVSLGERATINCRASESVDTYGSS | |
| | FVHWYQQKPGQPPKLLIYLASNLESGVPDRFSGSGSRTDFTLT | |
| | ISSLQAEDVATYYCQQNNEEPWTFGCGTKVEIK [SEQ ID | |
| | NO: 31] | |
| | GB100 (VL-VH): | |
| | DIVMTQSPASLAVSLGERATINCRASESVDTYGSSFVHWYQQK | |
| | PGQPPKLLIYLASNLESGVPDRFSGSGSRTDFTLTISSLQAED | |
| | VATYYCQQNNEEPWTFGCGTKVEIKGGGGSGGGGSGGGGSGGG | |
| | GSQVQLVQSGAEVKKPGASVKVSCKASGYTFTRYVMHWVRQAP | |
| | GQCLEWMGFINPYNDDTKYNEKFKGRVTITSDTSASTAYMELS | |
| | SLRSEDTAVYHCARWRQLGSLDSWGQGTTVTV [SEQ ID | |
| | NO: 32] | |
| Humanized | QVQLVQSGAEVKKPGASVKVS | DIVMTQSPASLAVSLGERATINC |
| 12H10.G7 | CKASGYTFTRYVMHWVRQAPG | RASESVDTYGSSFVHWYQQKPGQ |
| GB93/GB101 | QRLEWMGFINPYNDDTKYNEK | PPKLLIYLASNLESGVPDRFSGS |
| (back | FKGRVTITSDTSASTAYMELS | GSRTDFTLTISSLQAEDAAVYYC |
| mutations in | SLRSEDTAVYHCARWRQLGSL | QQNNEEPWTFGGGTKVEIK |
| VH and VL | DSWGQGTTVTVSS | [SEQ ID NO: 34] |
| underlined) | [SEQ ID NO: 9] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | GB93 (VH-VL): | |
| humanized | QVQLVQSGAEVKKPGASVKVSCKASGYTFTRYVMHWVRQAPGQ | |
| 12H10.G7 | CLEWMGFINPYNDDTKYNEKFKGRVTITSDTSASTAYMELSSL | |
| GB93/GB101 | RSEDTAVYHCARWRQLGSLDSWGQGTTVTVSSGGGGSGGGGSG | |
| | GGGSGGGGSDIVMTQSPASLAVSLGERATINCRASESVDTYGS | |
| | SFVHWYQQKPGQPPKLLIYLASNLESGVPDRFSGSGSRTDFTL | |
| | TISSLQAEDAAVYYCQQNNEEPWTFGCGTKVEIK [SEQ ID | |
| | NO: 35] | |
| | GB101 (VL-VH): | |
| | DIVMTQSPASLAVSLGERATINCRASESVDTYGSSFVHWYQQK | |
| | PGQPPKLLIYLASNLESGVPDRFSGSGSRTDFTLTISSLQAED | |
| | AAVYYCQQNNEEPWTFGCGTKVEIKGGGGSGGGGSGGGGSGGG | |
| | GSQVQLVQSGAEVKKPGASVKVSCKASGYTFTRYVMHWVRQAP | |
| | GQCLEWMGFINPYNDDTKYNEKFKGRVTITSDTSASTAYMELS | |
| | SLRSEDTAVYHCARWRQLGSLDSWGQGTTVTVSS [SEQ ID | |
| | NO: 36] | |
| Humanized | QVQLVQSGAEVKKPGASVKVS | DIVMTQSPDSLAVSLGERATINC |
| 12H10.G7 | CKASGYTFTRYVMHWVRQAPG | RASESVDTYGSSFVHWYQQKPGQ |
| GB94/GB102 | QRLEWMGFINPYNDDTKYNEK | PPKLLIYLASNLESGVPDRFSGS |
| | FKGRVTITRDTSASTAYMELS | GSGTDFTLTISSLQAEDVAVYYC |
| | SLRSEDTAVYYCARWRQLGSL | QQNNEEPWTFGGGTKVEIK |
| | DSWGQGTTVTVSS [SEQ ID | [SEQ ID NO: 38] |
| | NO: 37] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | GB94 (VH-VL): | |
| humanized | QVQLVQSGAEVKKPGASVKVSCKASGYTFTRYVMHWVRQAPGQ | |
| 12H10.G7 | CLEWMGFINPYNDDTKYNEKFKGRVTITRDTSASTAYMELSSL | |
| GB94/GB102 | RSEDTAVYYCARWRQLGSLDSWGQGTTVTVSSGGGGSGGGGSG | |
| | GGGSGGGGSDIVMTQSPDSLAVSLGERATINCRASESVDTYGS | |
| | SFVHWYQQKPGQPPKLLIYLASNLESGVPDRFSGSGSGTDFTL | |
| | TISSLQAEDVAVYYCQQNNEEPWTFGCGTKVEIK [SEQ ID | |
| | NO: 39] | |
| | GB102 (VL-VH): | |
| | DIVMTQSPDSLAVSLGERATINCRASESVDTYGSSFVHWYQQK | |
| | PGQPPKLLIYLASNLESGVPDRFSGSGSGTDFTLTISSLQAED | |
| | VAVYYCQQNNEEPWTFGCGTKVEIKGGGGSGGGGSGGGGSGGG | |
| | GSQVQLVQSGAEVKKPGASVKVSCKASGYTFTRYVMHWVRQAP | |
| | GQCLEWMGFINPYNDDTKYNEKFKGRVTITRDTSASTAYMELS | |
| | SLRSEDTAVYYCARWRQLGSLDSWGQGTTVTVSS [SEQ ID | |
| | NO: 40] | |
| Humanized | QVQLVQSGAEVKKPGASVKVS | DIVMTQSPDSLAVSLGERATINC |
| 12H10.G7 | CKASGYTFTRYVMHWVRQAPG | RASESVDTYGSSFVHWYQQKPGQ |
| GB102 | QRLEWMGFINPYNDDTKYNEK | PPKLLIYLASNLESGVPDRFSGS |
| D101E | FKGRVTITRDTSASTAYMELS | GSGTDFTLTISSLQAEDVAVYYC |
| | SLRSEDTAVYYCARWRQLGSL | QQNNEEPWTFGCGTKVEIK |
| | ESWGQGTTVTVSS [SEQ ID | [SEQ ID NO: 42] |
| | NO: 41] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | VH-VL: | |
| humanized | QVQLVQSGAEVKKPGASVKVSCKASGYTFTRYVMHWVRQAPGQ | |
| 12H10.G7 | CLEWMGFINPYNDDTKYNEKFKGRVTITRDTSASTAYMELSSL | |
| GB102 | RSEDTAVYYCARWRQLGSLESWGQGTTVTVSSGGGGSGGGGSG | |
| D101E | GGGSGGGGSDIVMTQSPDSLAVSLGERATINCRASESVDTYGS | |
| | SFVHWYQQKPGQPPKLLIYLASNLESGVPDRFSGSGSGTDFTL | |
| | TISSLQAEDVAVYYCQQNNEEPWTFGCGTKVEIK [SEQ ID | |
| | NO: 43] | |
| | VL-VH: | |
| | DIVMTQSPDSLAVSLGERATINCRASESVDTYGSSFVHWYQQK | |
| | PGQPPKLLIYLASNLESGVPDRFSGSGSGTDFTLTISSLQAED | |
| | VAVYYCQQNNEEPWTFGCGTKVEIKGGGGSGGGGSGGGGSGGG | |
| | GSQVQLVQSGAEVKKPGASVKVSCKASGYTFTRYVMHWVRQAP | |
| | GQCLEWMGFINPYNDDTKYNEKFKGRVTITRDTSASTAYMELS | |
| | SLRSEDTAVYYCARWRQLGSLESWGQGTTVTVSS [SEQ ID | |
| | NO: 44] | |
| Humanized | QVQLVQSGAEVKKPGASVKVS | DIVMTQSPDSLAVSLGERATINC |
| 12H10.G7 | CKASGYTFTRYVIHWVRQAPG | RASESVDTYGSSFVHWYQQKPGQ |
| GB102 M34I | QRLEWMGFINPYNDDTKYNEK | PPKLLIYLASNLESGVPDRFSGS |
| | FKGRVTITRDTSASTAYMELS | GSGTDFTLTISSLQAEDVAVYYC |
| | SLRSEDTAVYYCARWRQLGSL | QQNNEEPWTFGCGTKVEIK |
| | DSWGQGTTVTVSS [SEQ ID | [SEQ ID NO: 42] |
| | NO: 45] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLDS [SEQ | NO: 8] |
| | ID NO: 5] | |
| scFv of | VH-VL: | |
| humanized | QVQLVQSGAEVKKPGASVKVSCKASGYTFTRYVIHWVRQAPGQ | |
| 12H10.G7 | CLEWMGFINPYNDDTKYNEKFKGRVTITRDTSASTAYMELSSL | |
| GB102 M34I | RSEDTAVYYCARWRQLGSLDSWGQGTTVTVSSGGGGSGGGGSG | |
| | GGGSGGGGSDIVMTQSPDSLAVSLGERATINCRASESVDTYGS | |
| | SFVHWYQQKPGQPPKLLIYLASNLESGVPDRFSGSGSGTDFTL | |
| | TISSLQAEDVDIVMTQSPDSLAVSLGERATINCRASESVDTYG | |
| | SSFVHWYQQKPGAVYYCQQNNEEPWTFGCGTKVEIK [SEQ | |
| | ID NO: 47] | |
| | VL-VH: | |
| | QPPKLLIYLASNLESGVPDRFSGSGSGTDFTLTISSLQAEDVA | |
| | VYYCQQNNEEPWTFGCGTKVEIKGGGGSGGGGSGGGGSGGGGS | |
| | QVQLVQSGAEVKKPGASVKVSCKASGYTFTRYVIHWVRQAPGQ | |
| | CLEWMGFINPYNDDTKYNEKFKGRVTITRDTSASTAYMELSSL | |
| | RSEDTAVYYCARWRQLGSLDSWGQGTTVTVSS [SEQ ID | |
| | NO: 48] | |
| Humanized | QVQLVQSGAEVKKPGASVKVS | DIVMTQSPDSLAVSLGERATINC |
| 12H10.G7 | CKASGYTFTRYVIHWVRQAPG | RASESVDTYGSSFVHWYQQKPGQ |
| GB102 | QRLEWMGFINPYNDDTKYNEK | PPKLLIYLASNLESGVPDRFSGS |
| M34I/D101E | FKGRVTITRDTSASTAYMELS | GSGTDFTLTISSLQAEDVAVYYC |
| | SLRSEDTAVYYCARWRQLGSL | QQNNEEPWTFGCGTKVEIK |
| | ESWGQGTTVTVSS | [SEQ ID NO: 42] |
| | [SEQ ID NO: 49] | CDR1: RASESVDTYGSSFVH |
| | CDR1: GYTFTRY [SEQ ID | [SEQ ID NO: 6] |
| | NO: 11] | CDR2: LASNLES [SEQ ID |
| | CDR2: NPYNDD [SEQ ID | NO: 7] |
| | NO: 4] | CDR3: QQNNEEPWT [SEQ ID |
| | CDR3: WRQLGSLES [SEQ | NO: 8] |
| | ID NO: 50] | |
| scFv of | VH-VL: | |
| humanized | QVQLVQSGAEVKKPGASVKVSCKASGYTFTRYVIHWVRQAPGQC | |
| 12H10.G7 | LEWMGFINPYNDDTKYNEKFKGRVTITRDTSASTAYMELSSLRS | |
| GB102 | EDTAVYYCARWRQLGSLESWGQGTTVTVSSGGGGSGGGGSGGGG | |
| M34I/D101E | SGGGGSDIVMTQSPDSLAVSLGERATINCRASESVDTYGSSFVH | |
| | WYQQKPGQPPKLLIYLASNLESGVPDRFSGSGSGTDFTLTISSL | |
| | QAEDVAVYYCQQNNEEPWTFGCGTKVEIK [SEQ ID NO: | |
| | 51] | |
| | VL-VH: | |
| | DIVMTQSPDSLAVSLGERATINCRASESVDTYGSSFVHWYQQKP | |
| | GQPPKLLIYLASNLESGVPDRFSGSGSGTDFTLTISSLQAEDVA | |
| | VYYCQQNNEEPWTFGCGTKVEIKGGGGSGGGGSGGGGSGGGGSQ | |
| | VQLVQSGAEVKKPGASVKVSCKASGYTFTRYVIHWVRQAPGQCL | |
| | EWMGFINPYNDDTKYNEKFKGRVTITRDTSASTAYMELSSLRSE | |
| | DTAVYYCARWRQLGSLESWGQGTTVTVSS [SEQ ID NO: | |
| | 52] | |
| Humanized | QVQLVQSGAEVKKPGASVKVS | DIVMTQSPDSLAVSLGERATINC |
| 12H10.G7 | CKASGYTFTRYVX₁HWVRQAP | RASESVDTYGSSFVHWYQQKPGQ |
| consensus 1 | GQRLEWMGFINPYNDDTKYNE | PPKLLIYLASNLESGVPDRFSGS |
| | KFKGRVTITRDTSASTAYMEL | GSGTDFTLTISSLQAEDVAVYYC |
| | SSLRSEDTAVYYCARWRQLGS | QQNNEEPWTFGCGTKVEIK |
| | LX₂SWGQGTTVTVSS | [SEQ ID NO: 42] |
| | where X₁ is M or I, | CDR1: RASESVDTYGSSFVH |
| | and X₂ is E or D | [SEQ ID NO: 6] |
| | [SEQ ID NO: 53] | CDR2: LASNLES [SEQ ID |
| | CDR1: GYTFTRY [SEQ ID | NO: 7] |
| | NO: 11] | CDR3: QQNNEEPWT [SEQ ID |
| | CDR2: NPYNDD [SEQ ID | NO: 8] |
| | NO: 4] | |
| | CDR3: WRQLGSLXS, | |
| | where X is E or D | |
| | [SEQ ID NO: 55] | |
| Humanized | QVQLVQSGAEVKKPGASVKVS | DIVMTQSPX₁SLAVSLGERATIN |
| 12H10.G7 | CKASGYTFTRYVMHWVRQAPG | CRASESVDTYGSSFVHWYQQKPG |
| consensus 2 | QRLEWMGFINPYNDDTKYNEK | QPPKLLIYLASNLESGVPDRFSG |
| | FKGRVTITX₁DTSASTAYMEL | SGSX₂TDFTLTISSLQAEDX₃A |
| | SSLRSEDTAVYX₂CARWRQLG | X₄YYCQQNNEEPWTFGGGTKVEI |
| | SLDSWGQGTTVTVSS | K where X₁ is A or D, |
| | where X₁ is S or R, | X₂ is R or G, and X₃ is |
| | and X₂ is Y or H | A or V, and X₄ is T or |
| | [SEQ ID NO: 56] | V [SEQ ID NO: 57] |
| | CDR1: GYTFTRY [SEQ ID | CDR1: RASESVDTYGSSFVH |
| | NO: 11] | [SEQ ID NO: 6] |
| | CDR2: NPYNDD [SEQ ID | CDR2: LASNLES [SEQ ID |
| | NO: 4] | NO: 7] |
| | CDR3: WRQLGSLDS [SEQ | CDR3: QQNNEEPWT [SEQ ID |
| | ID NO: 5] | NO: 8] |
| Humanized | QVQLVQSGAEVKKPGASVKVS | DIVMTQSPDSLAVSLGERATINC |
| 12H10.G7 | CKASGYTFTRYVX₁HWVRQAP | RASESVDTYGSSFVHWYQQKPGQ |
| consensus 3 | GQCLEWMGFINPYNDDTKYNE | PPKLLIYLASNLESGVPDRFSGS |
| | KFKGRVTITRDTSASTAYMEL | GSGTDFTLTISSLQAEDVAVYYC |
| | SSLRSEDTAVYYCARWRQLGS | QQNNEEPWTFGCGTKVEIK |
| | LX₂SWGQGTTVTVSS | [SEQ ID NO: 42] |
| | where X₁ is M or I, | CDR1: RASESVDTYGSSFVH |
| | and X₂ is E or D | [SEQ ID NO: 6] |
| | [SEQ ID NO: 58] | CDR2: LASNLES [SEQ ID |
| | CDR1: GYTFTRY [SEQ ID | NO: 7] |
| | NO: 11] | CDR3: QQNNEEPWT [SEQ ID |
| | CDR2: NPYNDD [SEQ ID | NO: 8] |
| | NO: 4] | |
| | CDR3: WRQLGSLXS, | |
| | where X is E or D | |
| | [SEQ ID NO: 55] | |
| 14A5.E8 | EVQLQESGAELVQPGASVRLS | QIVLTQSPAIMSASPGEKVTMTC |
| | CKASGYTFTSYWINWVKQRPG | SASSSVSYMHWYQQKSGTSPKRW |
| | QGLEWIGNIYPGSSIINYNEN | IYDTSKLASGVPARFSGSGSGTS |
| | FKNRATLTVDTSSSTAYMQLS | YSLTISSMEAEDAATYYCQQWTS |
| | SLTSDDSAVYYCARRVVYLYF | KSPTFGGGTKLEIK [SEQ ID |
| | DYWGQGTTLTVSS [SEQ ID | NO: 61] |
| | NO: 60] | CDR1: SASSSVSYMH [SEQ |
| | CDR1: GYTFTSY [SEQ ID | ID NO: 65] |
| | NO: 62] | CDR2: DTSKLAS [SEQ ID |
| | CDR2: YPGSSI [SEQ ID | NO: 66] |
| | NO: 63] | CDR3: QQWTSKSPT [SEQ ID |
| | CDR3: RVVYLYFDY [SEQ | NO: 67] |
| | ID NO: 64] | |
| Humanized | QVQLVQSGAEVKKPGASVKVS | EIVLTQSPATLSLSPGEKATLSC |
| 14A5.E8 | CKVSGYTFTSYWINWVRQRPG | SASSSVSYMHWYQQKPGQAPRLL |
| 1551/1552 | KGLEWMGNIYPGSSIINYNEN | IYDTSKLASGIPARFSGSGSGTS |
| (back | FKNRVTMTVDTSSDTAYMELS | FTLTISSLEPEDAAVYYCQQWTS |
| mutations in | SLRSEDTAVYYCARRVVYLYF | KSPTFGGGTKVEIK [SEQ ID |
| VH and VL | DYWGQGTLVTVSS [SEQ ID | NO: 69] |
| underlined) | NO: 68] | CDR1: SASSSVSYMH [SEQ |
| | CDR1: GYTFTSY [SEQ ID | ID NO: 65] |
| | NO: 62] | CDR2: DTSKLAS [SEQ ID |
| | CDR2: YPGSSI [SEQ ID | NO: 66] |
| | NO: 63] | CDR3: QQWTSKSPT [SEQ |
| | CDR3: RVVYLYFDY [SEQ | ID NO: 67] |
| | ID NO: 64] | |
| scFv of | 1551 (VH-VL): | |
| humanized | QVQLVQSGAEVKKPGASVKVSCKVSGYTFTSYWINWVRQRPGKC | |
| 14A5.E8 | LEWMGNIYPGSSIINYNENFKNRVTMTVDTSSDTAYMELSSLRS | |
| 1551/1552 | EDTAVYYCARRVVYLYFDYWGQGTLVTVSSGGGGSGGGGSGGGG | |
| | SGGGGSEIVLTQSPATLSLSPGEKATLSCSASSSVSYMHWYQQK | |
| | PGQAPRLLIYDTSKLASGIPARFSGSGSGTSFTLTISSLEPEDA | |
| | AVYYCQQWTSKSPTFGCGTKVEIK [SEQ ID NO: 70] | |
| | 1552 (VL-VH): | |
| | EIVLTQSPATLSLSPGEKATLSCSASSSVSYMHWYQQKPGQAPR | |
| | LLIYDTSKLASGIPARFSGSGSGTSFTLTISSLEPEDAAVYYCQ | |
| | QWTSKSPTFGCGTKVEIKGGGGSGGGGSGGGGSGGGGSQVQLVQ | |
| | SGAEVKKPGASVKVSCKVSGYTFTSYWINWVRQRPGKCLEWMGN | |
| | IYPGSSIINYNENFKNRVTMTVDTSSDTAYMELSSLRSEDTAVY | |
| | YCARRVVYLYFDYWGQGTLVTVSS [SEQ ID NO: 71] | |
| Humanized | QVQLVQSGAEVKKPGASVKVS | EIVLTQSPATLSLSPGERATLSC |
| 14A5.E8 | CKVSGYTFTSYWINWVRQAPG | SASSSVSYMHWYQQKPGQAPRLL |
| 1553/1554 | KGLEWMGNIYPGSSIINYNEN | IYDTSKLASGIPARFSGSGSGTD |
| | FKNRVTMTEDTSTDTAYMELS | FTLTISSLEPEDFAVYYCQQWTS |
| | SLRSEDTAVYYCARRVVYLYF | KSPTFGGGTKVEIK [SEQ ID |
| | DYWGQGTLVTVSS [SEQ ID | NO: 73] |
| | NO: 72] | CDR1: SASSSVSYMH [SEQ |
| | CDR1: GYTFTSY [SEQ ID | ID NO: 65] |
| | NO: 62] | CDR2: DTSKLAS [SEQ ID |
| | CDR2: YPGSSI [SEQ ID | NO: 66] |
| | NO: 63] | CDR3: QQWTSKSPT [SEQ ID |
| | CDR3: RVVYLYFDY [SEQ | NO: 67] |
| | ID NO: 64] | |
| scFv of | 1553 (VH-VL): | |
| humanized | QVQLVQSGAEVKKPGASVKVSCKVSGYTFTSYWINWVRQAPGKC | |
| 14A5.E8 | LEWMGNIYPGSSIINYNENFKNRVTMTEDTSTDTAYMELSSLRS | |
| 1553/1554 | EDTAVYYCARRVVYLYFDYWGQGTLVTVSSGGGGSGGGGSGGGG | |
| | SGGGGSEIVLTQSPATLSLSPGERATLSCSASSSVSYMHWYQQK | |
| | PGQAPRLLIYDTSKLASGIPARFSGSGSGTDFTLTISSLEPEDF | |
| | AVYYCQQWTSKSPTFGCGTKVEIK [SEQ ID NO: 74] | |
| | 1554 (VL-VH): | |
| | EIVLTQSPATLSLSPGERATLSCSASSSVSYMHWYQQKPGQAPR | |
| | LLIYDTSKLASGIPARFSGSGSGTDFTLTISSLEPEDFAVYYCQ | |
| | QWTSKSPTFGCGTKVEIKGGGGSGGGGSGGGGSGGGGSQVQLVQ | |
| | SGAEVKKPGASVKVSCKVSGYTFTSYWINWVRQAPGKCLEWMGN | |
| | IYPGSSIINYNENFKNRVTMTEDTSTDTAYMELSSLRSEDTAVY | |
| | YCARRVVYLYFDYWGQGTLVTVSS [SEQ ID NO: 75] | |
| Humanized | QVQLVQSGAEVKKPGASVKVS | EIVLTQSPATLSLSPGERATLSC |
| 14A5.E8 | CKVSGYTFPYYWINWVRQAPG | SASSSVSYIHWYQQKPGQAPRLL |
| 1689 | KGLEWMGNIYPGSSIINYNEN | IYDTSKLASGIPARFSGSGSGTD |
| (affinity | FKNRVTMTEDTSTDTAYMELS | FTLTISSLEPEDFAVYYCQQWTS |
| matured from | SLRSEDTAVYYCARRNVYLTF | KSPTFGGGTKVEIK [SEQ ID |
| 1553) | DYWGQGTLVTVSS | NO: 77] |
| | [SEQ ID NO: 76] | CDR1: SASSSVSYIH [SEQ |
| | CDR1: GYTFPYY [SEQ ID | ID NO: 80] |
| | NO: 78] | CDR2: DTSKLAS [SEQ ID |
| | CDR2: YPGSSI [SEQ ID | NO: 66] |
| | NO: 63] | CDR3: QQWTSKSPT [SEQ ID |
| | CDR3: RNVYLTFDY [SEQ | NO: 67] |
| | ID NO: 79] | |
| scFv of | VH-VL: | |
| humanized | QVQLVQSGAEVKKPGASVKVSCKVSGYTFPYYWINWVRQAPGKC | |
| 14A5.E8 | LEWMGNIYPGSSIINYNENFKNRVTMTEDTSTDTAYMELSSLRS | |
| 1689 | EDTAVYYCARRNVYLTFDYWGQGTLVTVSSGGGGSGGGGSGGGG | |
| (affinity | SGGGGSEIVLTQSPATLSLSPGERATLSCSASSSVSYIHWYQQK | |
| matured from | PGQAPRLLIYDTSKLASGIPARFSGSGSGTDFTLTISSLEPEDF | |
| 1553) | AVYYCQQWTSKSPTFGCGTKVEIK [SEQ ID NO: 81] | |
| | VL-VH: | |
| | EIVLTQSPATLSLSPGERATLSCSASSSVSYIHWYQQKPGQAPR | |
| | LLIYDTSKLASGIPARFSGSGSGTDFTLTISSLEPEDFAVYYCQ | |
| | QWTSKSPTFGCGTKVEIKGGGGSGGGGSGGGGSGGGGSQVQLVQ | |
| | SGAEVKKPGASVKVSCKVSGYTFPYYWINWVRQAPGKCLEWMGN | |
| | IYPGSSIINYNENFKNRVTMTEDTSTDTAYMELSSLRSEDTAVY | |
| | YCARRNVYLTFDYWGQGTLVTVSS [SEQ ID NO: 82] | |
| Humanized | QVQLVQSGAEVKKPGASVKVS | EIVLTQSPATLSLSPGERATLSC |
| 14A5.E8 | CKVSGYTFX₁X₂YWINWVRQ | SASSSVSYXHWYQQKPGQAPRLL |
| consensus | X₃PGKX₄LEWMGNIYPGSSII | IYDTSKLASGIPARFSGSGSGTD |
| | NYNENFKNRVTMTX₅DTSX₆D | FTLTISSLEPEDFAVYYCQQWTS |
| | TAYMELSSLRSEDTAVYYCAR | KSPTFGGGTKVEIK |
| | RX₇VYLX₈FDYWGQGTLVTVS | where X is M or I |
| | S where X₁ is P or T, | [SEQ ID NO: 84] |
| | X₂ is S or Y, X₃ is | CDR1: SASSSVSYXH, |
| | A or R, X₄ is C or G, | wherein X is M or I |
| | X₅ is V or E, X₆ is S | [SEQ ID NO: 86] |
| | or T, X₇ is N or V, | CDR2: DTSKLAS [SEQ ID |
| | and X₈ is T or Y [SEQ | NO: 66] |
| | ID NO: 29] | CDR3: QQWTSKSPT [SEQ ID |
| | CDR1: GYTFX₁X₂Y, | NO: 67] |
| | where X₁ is P or T, | |
| | and X₂ is S or Y [SEQ | |
| | ID NO: 59] | |
| | CDR2: YPGSSI [SEQ ID | |
| | NO: 63] | |
| | CDR3: RX₁VYLX₂FDY, | |
| | where X₁ is N or V, | |
| | and X₂ is T or Y [SEQ | |
| | ID NO: 54] | |
| 11F4.B9 | EVQLQESGPELVKPGASVKIS | DIVLTQSPASLAVSLGQRATISC |
| | CKASGYSFTGYYIHWVKQGPE | RASESVDIYGNSFMHWYQQKPGQ |
| | KSLEWIGEIIPSTGSTIYNQK | PPKLLIYRASNLESGIPARFSGS |
| | FKAKATLTVDKSSSTAYLQLK | GSRTDFTLTINPVEADDVATYYC |
| | SLTSEDSAVYYCERWGDYYGR | QQSNEDPRTFGGGTKLEIK |
| | DYWGQGTSVTVSS | [SEQ ID NO: 90] |
| | [SEQ ID NO: 85] | CDR1: RASESVDIYGNSFMH |
| | CDR1: GYSFTGY [SEQ ID | [SEQ ID NO: 91] |
| | NO: 87] | CDR2: RASNLES [SEQ ID |
| | CDR2: IPSTGS [SEQ ID | NO: 92] |
| | NO: 88] | CDR3: QQSNEDPRT [SEQ ID |
| | CDR3: WGDYYGRDY [SEQ | NO: 93] |
| | ID NO: 89] | |
| Humanized | QVQLVQSGAEVKKPGASVKVS | DIVMTQSPASLAVSLGERATINC |
| 11F4.B9 | CKASGYSFTGYYIHWVRQGPG | RASESVDIYGNSFMHWYQQKPGQ |
| (back | QGLEWMGEIIPSTGSTIYAQK | PPKLLIYRASNLESGVPDRFSGS |
| mutations | FQGRVTMTRDTSTSTVYMELS | GSRTDFTLTINSLQAEDVATYYC |
| underlined) | SLRSEDTAVYYCERWGDYYGR | QQSNEDPRTFGGGTKVEIK |
| | DYWGQGTLVTVSS | [SEQ ID NO: 94] |
| | [SEQ ID NO: 14] | CDR1: RASESVDIYGNSFMH |
| | CDR1: GYSFTGY [SEQ ID | [SEQ ID NO: 91] |
| | NO: 87] | CDR2: RASNLES [SEQ ID |
| | CDR2: IPSTGS [SEQ ID | NO: 92] |
| | NO: 88] | CDR3: QQSNEDPRT [SEQ ID |
| | CDR3: WGDYYGRDY [SEQ | NO: 93] |
| | ID NO: 89] | |
| 4A4.A3 | QVTLKESGPGILQPSQTLSLT | RIVMTQSPTTMAASPGEKITITC |
| | CSFSGFSLTTYGMGVGWIRQP | SASSSISSIYLHWYQQKPGFSPK |
| | SGKGLEWLANIWFNDNKYYNS | LLIFRTSDLASGVPPRFGGSGSG |
| | TLKSRLTISKDTSNNQVFLKI | TSYSLTIGTMEAEDVATYYCQQG |
| | SSVDTTDTATYYCAQITTVVG | SSFPRTFGGGTKLEIK [SEQ |
| | TFDYWGQGSPLTVSP [SEQ | ID NO: 96] |
| | ID NO: 95] | CDR1: SASSSISSIYLH [SEQ |
| | CDR1: GFSLTTYGM [SEQ | ID NO: 101] |
| | ID NO: 97] | CDR2: RTSDLAS [SEQ ID |
| | CDR2: WFNDN [SEQ ID | NO: 102] |
| | NO: 99] | CDR3: QQGSSFPRT [SEQ ID |
| | CDR3: ITTVVGTFDY [SEQ | NO: 103] |
| | ID NO: 100] | |
| 4A4.H7 | EVQLQESGPELVKPGASVKIS | DIVLTQSPASLAVSLGQRATISC |
| | CKASGYSFTGYYIHWVKQSPE | RASETVDTHGNSFMHWYQQKPGQ |
| | ESLEWIGEIYPNTGITTYNQK | PPKLLIYRASNLESGIPARFSGS |
| | FTAKATLTVDKSSNTAYMQLK | GSRTDFTLTINPVEADDVATYYC |
| | SLTSEDSAVYYCTRWGDYYGR | QQSNEDPRTFGGGTKLEIK |
| | DYWGQGTSVTVSS | [SEQ ID NO: 105] |
| | [SEQ ID NO: 104] | CDR1: RASETVDTHGNSFMH |
| | CDR1: GYSFTGY [SEQ ID | [SEQ ID NO: 106] |
| | NO: 87] | CDR2: RASNLES [SEQ ID |
| | CDR2: YPNTGI [SEQ ID | NO: 92] |
| | NO: 98] | CDR3: QQSNEDPRT [SEQ |
| | CDR3: WGDYYGRDY [SEQ | ID NO: 93] |
| | ID NO: 89] | |
| 15A11.C8 | EVQLQESGGGLVKTGGSRKLS | DIQMTQTTSSLSASLGDRVTIRC |
| | CAASGFTFSDYGMHWVRHTPE | RASQDITNYLNWYQQKPDGAVKL |
| | KGLEWVVYISSGGNTIFYTDT | LISYTSILQSGVPSRFSGSGSGT |
| | VKGRFTISRDNAKNTLFLQMT | DYSLTISNLEQGDVATYFCQQGS |
| | SLRSEDTAVYFCVRQGYYYAM | SLPWTFGGGTKLEIK [SEQ ID |
| | DYWGQGASVTVSS [SEQ ID | NO: 108] |
| | NO: 107] | CDR1: RASQDITNYLN [SEQ |
| | CDR1: GFTFSDY [SEQ ID | ID NO: 112] |
| | NO: 109] | CDR2: YTSILQS [SEQ ID |
| | CDR2: SSGGNT [SEQ ID | NO: 113] |
| | NO: 110] | CDR3: QQGSSLPWT [SEQ ID |
| | CDR3: QGYYYAMDY [SEQ | NO: 114] |
| | ID NO: 111] | |
| 12C9.E5 | EVQLQESGAELVRPGASVKLS | DVVMTQTPLSLSVTIGQPASISC |
| | CKASGYIFTDYEIHWVKQTPV | KSSQSLLYSDGETYLNWLQQRPG |
| | HGLEWIGAIDPETGITAYSQK | QSPKRLMYQVSKLDPGIPDRFSG |
| | FKGKATLTTDTSSSTAYMEFR | SGSETDFTLKISRVEAEDLGIYY |
| | SLTSEDSAVYYCTRGGLLYWG | CLQGTFYPHTFGGGTKLEIK |
| | QGTSVTVSS | [SEQ ID NO: 116] |
| | [SEQ ID NO: 115] | CDR1: KSSQSLLYSDGETYLN |
| | CDR1: GYIFTDY [SEQ ID | [SEQ ID NO: 120] |
| | NO: 117] | CDR2: QVSKLDP [SEQ ID |
| | CDR2: DPETGI [SEQ ID | NO: 121] |
| | NO: 118] | CDR3: LQGTFYPHT [SEQ ID |
| | CDR3: GGLLY [SEQ ID | NO: 122] |
| | NO: 119] | |
| 1A2.A3 | EVQLQESGPELVKPGASVKIS | DIVLTQSPASLAVSLGQRATISC |
| | CKASGYSFTGYYIHWVKQSPE | RASETVDTHGNSFMHWYQQKPGQ |
| | ESLEWIGEIYPNTGITTYNQK | PPKLLIYRASNLESGIPARFSGS |
| | FTAKATLTVDKSSNTAYMQLK | GSRTDFTLTINPVEADDVATYYC |
| | SLTSEDSAVYYCTRWGDYYGR | QQSNEDPRTFGGGTKLEIK |
| | DYWGQGTSVTVSS | [SEQ ID NO: 124] |
| | [SEQ ID NO: 123] | CDR1: RASETVDTHGNSFMH |
| | CDR1: GYSFTGY [SEQ ID | [SEQ ID NO: 106] |
| | NO: 87] | CDR2: RASNLES [SEQ ID |
| | CDR2: YPNTGI [SEQ ID | NO: 92] |
| | NO: 98] | CDR3: QQSNEDPRT [SEQ ID |
| | CDR3: WGDYYGRDY [SEQ | NO: 93] |
| | ID NO: 89] | |
| 4H2.E3 | EVQLQESGPELVKPGASVKMS | GIVMTQTTPSVPVTPGESVSISC |
| | CKASGYTFTSYLMHWMKQKPG | RSSKSLLHSNGNTYLYWFLQRPG |
| | QGLEWIGYINPYSDGIKYNEK | QSPQLLIYRMSNLASGVPDRFSG |
| | FRDKATLTSDKSSNTAYMELS | SGSGTTFTLRISRVEAEDVGVYY |
| | SLTSEDSAVYYCAHSSGYVGY | CMQHLEYPFTFGSGTKLEIK |
| | AMDYWGQGTSVTVSS | [SEQ ID NO: 126] |
| | [SEQ ID NO: 125] | |
| | CDR1: GYTFTSY [SEQ ID | CDR1: RSSKSLLHSNGNTYLY |
| | NO: 62] | [SEQ ID NO: 128] |
| | CDR2: NPYSDG [SEQ ID | CDR2: RMSNLAS [SEQ ID |
| | NO: 33] | NO: 129] |
| | CDR3: SSGYVGYAMDY | CDR3: MQHLEYPFT [SEQ ID |
| | [SEQ ID NO: 127] | NO: 130] |
| 14H8.E7 | EVQLQESGAELVKPGASVKLS | QIVLTQSPAIMSASPGEKVTMTC |
| | CKASGYTFTNYWINWLKQRPG | SASSSVSYMHWYQQKSGTSPKRW |
| | QGLEWIGNIYPGSTIINYNEK | IFDTSKLASGVPVRFSGSGSGTS |
| | FKNKATLTVDTSSSTAYMQLS | YSLTITNMETEDAATYYCQQWSS |
| | SLTSDDSAVYYCARRVVYLYF | KSPTFGGGTKLEIK [SEQ ID |
| | DSWGQGTTLTVSS | NO: 83] |
| | [SEQ ID NO: 131] | CDR1: SASSSVSYMH [SEQ |
| | CDR1: GYTFTNY [SEQ ID | ID NO: 65] |
| | NO: 132] | CDR2: DTSKLAS [SEQ ID |
| | CDR2: YPGSTI [SEQ ID | NO: 66] |
| | NO: 133] | CDR3: QQWSSKSPT [SEQ ID |
| | CDR3: RVVYLYFDS [SEQ | NO: 46] |
| | ID NO: 134] | |

Alternatively, novel antigen-binding sites that can bind to FLT3 can be identified by screening for binding to the amino acid sequence defined by SEQ ID NO:135, a mature extracellular fragment thereof, or a fragment containing a domain of FLT3 (see, e.g., International Application Publication No. WO 2018/220584).

(mature human FLT3 extracellular domain)
SEQ ID NO: 135
NQDLPVIKCVLINHKNNDSSVGKSSSYPMVSESPEDLGCALRPQSSGTVY
EAAAVEVDVSASITLQVLVDAPGNISCLWVFKHSSLNCQPHFDLQNRGVV
SMVILKMTETQAGEYLLFIQSEATNYTILFTVSIRNTLLYTLRRPYFRKM
ENQDALVCISESVPEPIVEWVLCDSQGESCKEESPAVVKKEEKVLHELFG
TDIRCCARNELGRECTRLFTIDLNQTPQTTLPQLFLKVGEPLWIRCKAVH
VNHGFGLTWELENKALEEGNYFEMSTYSTNRTMIRILFAFVSSVARNDTG
YYTCSSSKHPSQSALVTIVEKGFINATNSSEDYEIDQYEEFCFSVRFKAY
PQIRCTWTFSRKSFPCEQKGLDNGYSISKFCNHKHQPGEYIFHAENDDAQ
FTKMFTLNIRRKPQVLAEASASQASCFSDGYPLPSWTWKKCSDKSPNCTE
EITEGVWNRKANRKVFGQWVSSSTLNMSEAIKGFLVKCCAYNSLGTSCET
ILLNSPGPFPFIQDNIS

In certain embodiments, the second antigen-binding site that binds FLT3 (e.g., human FLT3) comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 2, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VH of the same antibody disclosed in Table 2. In certain embodiments, the second antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antibody discloses in Table 2. In certain embodiments, the second antigen-binding site comprises the heavy chain CDR1, CDR2, and CDR3 and the light chain CDR1, CDR2, and CDR3 of an antibody disclosed in Table 2.

In certain embodiments, the second antigen-binding site is related to 12H10.G7. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:1, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:2. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively.

In certain embodiments, the second antigen-binding site is related to GB87 or GB95. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 3 or 12.

In certain embodiments, the second antigen-binding site is related to GB88 or GB96. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:13, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 15 or 16.

In certain embodiments, the second antigen-binding site is related to GB89 or GB97. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:17, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 19 or 20.

In certain embodiments, the second antigen-binding site is related to GB90 and GB98. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:22. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 23 or 24.

In certain embodiments, the second antigen-binding site is related to GB91 and GB99. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:26. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 27 or 28.

In certain embodiments, the second antigen-binding site is related to GB92 or GB100. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:30. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 31 or 32.

In certain embodiments, the second antigen-binding site is related to GB93 or GB101. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:34. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 35 or 36.

In certain embodiments, the second antigen-binding site is related to GB94 or GB102. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:37, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:38. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 39 or 40.

In certain embodiments, the second antigen-binding site is related to GB102 D101E. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:41, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 50, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 43 or 44.

In certain embodiments, the second antigen-binding site is related to GB102 M34I. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:45, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 47 or 48.

In certain embodiments, the second antigen-binding site is related to GB102 M34I/D101E. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:49, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 50, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 50, respectively; and (b) a VL that comprises CDR1, CDR2, and CDR3 comprising the amino acid sequences of SEQ ID NOs: 6, 7, and 8, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 51 or 52.

In certain embodiments, the second antigen-binding site is related to humanized 12H10.G7. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:53, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 55, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 55, respectively; and (b) a VL that comprises CDR1, CDR2, and CDR3 comprising the amino acid sequences of SEQ ID NOs: 6, 7, and 8, respectively.

In certain embodiments, the second antigen-binding site is related to humanized 12H10.G7. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:56, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:57. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively.

In certain embodiments, the second antigen-binding site is related to humanized 12H10.G7. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:58, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 55, respectively. In certain embodiments, the VL comprises CDR1, CDR2, and CDR3 comprising the amino acid sequences of SEQ ID NOs: 6, 7, and 8, respectively. In certain embodiments, the second antigen-binding site comprises (a) a VH that comprises CDR1, CDR2, and CDR3 comprising the amino acid sequences of SEQ ID NOs: 11, 4, and 55, respectively; and (b) a VL that comprises CDR1, CDR2, and CDR3 comprising the amino acid sequences of SEQ ID NOs: 6, 7, and 8, respectively.

In certain embodiments, the second antigen-binding site is related to 14A5.E8. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:60, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:61. In certain embodiments, the VH comprises CDR1, CDR2, and CDR3 comprising the amino acid sequences of SEQ ID NOs: 62, 63, and 64, respectively. In certain embodiments, the VL comprises CDR1, CDR2, and CDR3 comprising the amino acid sequences of SEQ ID NOs: 65, 66, and 67, respectively. In certain embodiments, the second antigen-binding site comprises (a) a VH that comprises CDR1, CDR2, and CDR3 comprising the amino acid sequences of SEQ ID NOs: 62, 63, and 64, respectively; and (b) a VL that comprises CDR1, CDR2, and CDR3 comprising the amino acid sequences of SEQ ID NOs: 65, 66, and 67, respectively.

In certain embodiments, the second antigen-binding site is related to mAb 1551 or 1552. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:68, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:69. In certain embodiments, the VH comprises CDR1, CDR2, and CDR3 comprising the amino acid sequences of SEQ ID NOs: 62, 63, and 64, respectively. In certain embodiments, the VL comprises CDR1, CDR2, and CDR3 comprising the amino acid sequences of SEQ ID NOs: 65, 66, and 67, respectively. In certain embodiments, the second antigen-binding site comprises (a) a VH that comprises CDR1, CDR2, and CDR3 comprising the amino acid sequences of SEQ ID NOs: 62, 63, and 64, respectively; and (b) a VL that comprises CDR1, CDR2, and CDR3 comprising the amino acid sequences of SEQ ID NOs: 65, 66, and 67, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 70 or 71.

In certain embodiments, the second antigen-binding site is related to mAb 1553 or 1554. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:72, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:73. In certain embodiments, the VH comprises CDR1, CDR2, and CDR3 comprising the amino acid sequences of SEQ ID NOs: 62, 63, and 64, respectively. In certain embodiments, the VL comprises CDR1, CDR2, and CDR3 comprising the amino acid sequences of SEQ ID NOs: 65, 66, and 67, respectively. In certain embodiments, the second antigen-binding site comprises (a) a VH that comprises CDR1, CDR2, and CDR3 comprising the amino acid sequences of SEQ ID NOs: 62, 63, and 64, respectively; and (b) a VL that comprises CDR1, CDR2, and CDR3 comprising the amino acid sequences of SEQ ID NOs: 65, 66, and 67, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 74 or 75.

In certain embodiments, the second antigen-binding site is related to mAb 1689. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:76, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:77. In certain embodiments, the VH comprises CDR1, CDR2, and CDR3 comprising the amino acid sequences of SEQ ID NOs: 78, 63, and 79, respectively. In certain embodiments, the VL comprises CDR1, CDR2, and CDR3 comprising the amino acid sequences of SEQ ID NOs: 80, 66, and 67, respectively. In certain embodiments, the second antigen-binding site comprises (a) a VH that comprises CDR1, CDR2, and CDR3 comprising the amino acid sequences of SEQ ID NOs: 78, 63, and 79, respectively; and (b) a VL that comprises CDR1, CDR2, and CDR3 comprising the amino acid sequences of SEQ ID NOs: 80, 66, and 67, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 81 or 82.

In certain embodiments, the second antigen-binding site is related to humanized 14A5.E8. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:29, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:84. In certain embodiments, the VH comprises CDR1, CDR2, and CDR3 comprising the amino acid sequences of SEQ ID NOs: 59, 63, and 54, respectively. In certain embodiments, the VL comprises CDR1, CDR2, and CDR3 comprising the amino acid sequences of SEQ ID NOs: 86, 66, and 67, respectively. In certain embodiments, the second antigen-binding site comprises (a) a VH that comprises CDR1, CDR2, and CDR3 comprising the amino acid sequences of SEQ ID NOs: 59, 63, and 54, respectively; and (b) a VL that comprises CDR1, CDR2, and CDR3 comprising the amino acid sequences of SEQ ID NOs: 86, 66, and 67, respectively.

In certain embodiments, the second antigen-binding site is related to 11F4.B9. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:85, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:90. In certain embodiments, the VH comprises CDR1, CDR2, and CDR3 comprising the amino acid sequences of SEQ ID NOs: 87, 88, and 89, respectively. In certain embodiments, the VL comprises CDR1, CDR2, and CDR3 comprising the amino acid sequences of SEQ ID NOs: 91, 92, and 93, respectively. In certain embodiments, the second antigen-binding site comprises (a) a VH that comprises CDR1, CDR2, and CDR3 comprising the amino acid sequences of SEQ ID NOs: 87, 88, and 89, respectively; and (b) a VL that comprises CDR1, CDR2, and CDR3 comprising the amino acid sequences of SEQ ID NOs: 91, 92, and 93, respectively.

In certain embodiments, the second antigen-binding site is related to humanized 11F4.B9. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:14, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:94. In certain embodiments, the VH comprises CDR1, CDR2, and CDR3 comprising the amino acid sequences of SEQ ID NOs: 87, 88, and 89, respectively. In certain embodiments, the VL comprises CDR1, CDR2, and CDR3 comprising the amino acid sequences of SEQ ID NOs: 91, 92, and 93, respectively. In certain embodiments, the second antigen-binding site comprises (a) a VH that comprises CDR1, CDR2, and CDR3 comprising the amino acid sequences of SEQ ID NOs: 87, 88, and 89, respectively; and (b) a VL that comprises CDR1, CDR2, and CDR3 comprising the amino acid sequences of SEQ ID NOs: 91, 92, and 93, respectively.

In certain embodiments, the second antigen-binding site is related to 4A4.A3. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:95, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:96. In certain embodiments, the VH comprises CDR1, CDR2, and CDR3 comprising the amino acid sequences of SEQ ID NOs: 97, 99, and 100, respectively. In certain embodiments, the VL comprises CDR1, CDR2, and CDR3 comprising the amino acid sequences of SEQ ID NOs: 101, 102, and 103, respectively. In certain embodiments, the second antigen-binding site comprises (a) a VH that comprises CDR1, CDR2, and CDR3 comprising the amino acid sequences of SEQ ID NOs: 97, 99, and 100, respectively; and (b) a VL that comprises CDR1, CDR2, and CDR3 comprising the amino acid sequences of SEQ ID NOs: 101, 102, and 103, respectively.

In certain embodiments, the second antigen-binding site is related to 4A4.H7. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:104, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:105. In certain embodiments, the VH comprises CDR1, CDR2, and CDR3 comprising the amino acid sequences of SEQ ID NOs: 87, 98, and 89, respectively. In certain embodiments, the VL comprises CDR1, CDR2, and CDR3 comprising the amino acid sequences of SEQ ID NOs: 106, 92, and 93, respectively. In certain embodiments, the second antigen-binding site comprises (a) a VH that comprises CDR1, CDR2, and CDR3 comprising the amino acid sequences of SEQ ID NOs: 87, 98, and 89, respectively; and (b) a VL that comprises CDR1, CDR2, and CDR3 comprising the amino acid sequences of SEQ ID NOs: 106, 92, and 93, respectively.

In certain embodiments, the second antigen-binding site is related to 15A11.C8. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:107, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:108. In certain embodiments, the VH comprises CDR1, CDR2, and CDR3 comprising the amino acid sequences of SEQ ID NOs: 109, 110, and 111, respectively. In certain embodiments, the VL comprises CDR1, CDR2, and CDR3 comprising the amino acid sequences of SEQ ID NOs: 112, 113, and 114, respectively. In certain embodiments, the second antigen-binding site comprises (a) a VH that comprises CDR1, CDR2, and CDR3 comprising the amino acid sequences of SEQ ID NOs: 109, 110, and 111, respectively; and (b) a VL that comprises CDR1, CDR2, and CDR3 comprising the amino acid sequences of SEQ ID NOs: 112, 113, and 114, respectively.

In certain embodiments, the second antigen-binding site is related to 12C9.E5. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:115, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:116. In certain embodiments, the VH comprises CDR1, CDR2, and CDR3 comprising the amino acid sequences of SEQ ID NOs: 117, 118, and 119, respectively. In certain embodiments, the VL comprises CDR1, CDR2, and CDR3 comprising the amino acid sequences of SEQ ID NOs: 120, 121, and 122, respectively. In certain embodiments, the second antigen-binding site comprises (a) a VH that comprises CDR1, CDR2, and CDR3 comprising the amino acid sequences of SEQ ID NOs: 117, 118, and 119, respectively; and (b) a VL that comprises CDR1, CDR2, and CDR3 comprising the amino acid sequences of SEQ ID NOs: 120, 121, and 122, respectively.

In certain embodiments, the second antigen-binding site is related to 1A2.A3. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:123, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:124. In certain embodiments, the VH comprises CDR1, CDR2, and CDR3 comprising the amino acid sequences of SEQ ID NOs: 87, 98, 89, respectively. In certain embodiments, the VL comprises CDR1, CDR2, and CDR3 comprising the amino acid sequences of SEQ ID NOs: 106, 92, 93, respectively. In certain embodiments, the second antigen-binding site comprises (a) a VH that comprises CDR1, CDR2, and CDR3 comprising the amino acid sequences of SEQ ID NOs: 87, 98, 89, respectively; and (b) a VL that comprises CDR1, CDR2, and CDR3 comprising the amino acid sequences of SEQ ID NOs: 106, 92, 93, respectively.

In certain embodiments, the second antigen-binding site is related to 4H2.E3. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:125, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:126. In certain embodiments, the VH comprises CDR1, CDR2, and CDR3 comprising the amino acid sequences of SEQ ID NOs: 62, 33, and 127, respectively. In certain embodiments, the VL comprises CDR1, CDR2, and CDR3 comprising the amino acid sequences of SEQ ID NOs: 128, 129, and 130, respectively. In certain embodiments, the second antigen-binding site comprises (a) a VH that comprises CDR1, CDR2, and CDR3 comprising the amino acid sequences of SEQ ID NOs: 62, 33, and 127, respectively; and (b) a VL that comprises CDR1, CDR2, and CDR3 comprising the amino acid sequences of SEQ ID NOs: 128, 129, and 130, respectively.

In certain embodiments, the second antigen-binding site is related to 14H8.E7. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:131, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:83. In certain embodiments, the VH comprises CDR1, CDR2, and CDR3 comprising the amino acid sequences of SEQ ID NOs: 132, 133, and 134, respectively. In certain embodiments, the VL comprises CDR1, CDR2, and CDR3 comprising the amino acid sequences of SEQ ID NOs: 65, 66, and 46, respectively. In certain embodiments, the second antigen-binding site comprises (a) a VH that comprises CDR1, CDR2, and CDR3 comprising the amino acid sequences of SEQ ID NOs: 132, 133, and 134, respectively; and (b) a VL that comprises CDR1, CDR2, and CDR3 comprising the amino acid sequences of SEQ ID NOs: 65, 66, and 46, respectively.

In each of the foregoing embodiments, it is contemplated herein that the VH and/or VL sequences that together bind FLT3 may contain amino acid alterations (e.g., at least 1, 2, 3, 4, 5, or 10 amino acid substitutions, deletions, or additions) in the framework regions of the VH and/or VL without affecting their ability to bind to FLT3 significantly.

In certain embodiments, a second antigen-binding site disclosed herein binds FLT3 (e.g., human FLT3) with a K_{D} (i.e., dissociation constant) of 1 nM or lower, 5 nM or lower, or 10 nM or lower, 15 nM or lower, or 20 nM or lower, as measured by surface plasmon resonance (SPR) (e.g., using the method described in Example 1 infra) or by bio-layer interferometry (BLI), and/or binds FLT3 from a body fluid, tissue, and/or cell of a subject. In certain embodiments, any of the foregoing isolated antibodies has a K_{d} (i.e., off-rate, also called K_{off}) equal to or lower than 1×10⁻⁵, 1×10⁻⁴, 1×10⁻³, 5×10⁻³, 0.01, 0.02, or 0.05 1/s, as measured by SPR (e.g., using the method described in Example 1 infra) or by BLI.

In certain embodiments, a second antigen-binding site disclosed herein, e.g., an antigen-binding site related to 12H10.G7, GB87, GB88, GB89, GB90, GB91, GB92, GB93, GB94, GB95, GB96, GB97, GB98, GB99, GB100, GB101, GB102, GB102 M34I, GB102 D101E, GB102 M34I/D101E, or a humanized 12H10.G7 disclosed above, binds a human FLT3 variant having a T227M mutation or the extracellular region thereof. The amino acid sequence of the extracellular region of hFLT3-T227M is

(SEQ ID NO: 318)
NQDLPVIKCVLINHKNNDSSVGKSSSYPMVSESPEDLGCALRPQSSGTVY
EAAAVEVDVSASITLQVLVDAPGNISCLWVFKHSSLNCQPHFDLQNRGVV
SMVILKMTETQAGEYLLFIQSEATNYTILFTVSIRNTLLYTLRRPYFRKM
ENQDALVCISESVPEPIVEWVLCDSQGESCKEESPAVVKKEEKVLHELFG
MDIRCCARNELGRECTRLFTIDLNQTPQTTLPQLFLKVGEPLWIRCKAVH
VNHGFGLTWELENKALEEGNYFEMSTYSTNRTMIRILFAFVSSVARNDTG
YYTCSSSKHPSQSALVTIVEKGFINATNSSEDYEIDQYEEFCFSVRFKAY
PQIRCTWTFSRKSFPCEQKGLDNGYSISKFCNHKHQPGEYIFHAENDDAQ
FTKMFTLNIRRKPQVLAEASASQASCFSDGYPLPSWTWKKCSDKSPNCTE
EITEGVWNRKANRKVFGQWVSSSTLNMSEAIKGFLVKCCAYNSLGTSCET
ILLNSPGPFPFIQDNIS.

In certain embodiments, a second antigen-binding site disclosed herein, e.g., an antigen-binding site related to 12H10.G7, GB87, GB88, GB89, GB90, GB91, GB92, GB93, GB94, GB95, GB96, GB97, GB98, GB99, GB100, GB101, GB102, GB102 M34I, GB102 D101E, GB102 M34I/D101E, or a humanized 12H10.G7 disclosed above, binds a human FLT3 variant having an ITD mutation or the extracellular region thereof. The amino acid sequence of the extracellular region of hFLT3-ITD 15

(SEQ ID NO: 319)
NQDLPVIKCVLINHKNNDSSVGKSSSYPMVSESPEDLGCALRPQSSGTVY
EAAAVEVDVSASITLQVLVDAPGNISCLWVFKHSSLNCQPHFDLQNRGVV
SMVILKMTETQAGEYLLFIQSEATNYTILFTVSIRNTLLYTLRRPYFRKM
ENQDALVCISESVPEPIVEWVLCDSQGESCKEESPAVVKKEEKVLHELFG
TDIRCCARNELGRECTRLFTIDLNQTPQTTLPQLFLKVGEPLWIRCKAVH
VNHGFGLTWELENKALEEGNYFEMSTYSTNRTMIRILFAFVSSVARNDTG
YYTCSSSKHPSQSALVTIVEKGFINATNSSEDYEIDQYEEFCFSVRFKAY
PQIRCTWTFSRKSFPCEQKGLDNGYSISKFCNHKHQPGEYIFHAENDDAQ
FTKMFTLNIRRKPQVLAEASASQASCFSDGYPLPSWTWKKCSDKSPNCTE
EITEGVWNRKANRKVFGQWVSSSTLNMSEAIKGFLVKCCAYNSLGTSCET
ILLNSPGPFPFIQDNIS.

In certain embodiments, a second antigen-binding site disclosed herein, e.g., an antigen-binding site related to 12H10.G7, GB87, GB88, GB89, GB90, GB91, GB92, GB93, GB94, GB95, GB96, GB97, GB98, GB99, GB100, GB101, GB102, GB102 M34I, GB102 D101E, GB102 M34I/D101E, a humanized 12H10.G7, 14A5.E8, 1551, 1552, 1553, 1554, 1689, a humanized 14A5.E8, 11F4.B9, 4A4.A3, 4A4.H7, 15A11.C8, 1A2.A3, 4H2.E3, or 14H8.E7 disclosed above, binds cynomolgus FLT3.

In certain embodiments, a second antigen-binding site disclosed herein, e.g., an antigen-binding site related to 12H10.G7, GB87, GB88, GB89, GB90, GB91, GB92, GB93, GB94, GB95, GB96, GB97, GB98, GB99, GB100, GB101, GB102, GB102 M34I, GB102 D101E, GB102 M34I/D101E, a humanized 12H10.G7, 14A5.E8, 1551, 1552, 1553, 1554, 1689, a humanized 14A5.E8, 11F4.B9, 4A4.A3, 4A4.H7, 12C9.E5, 1A2.A3, 4H2.E3, or 14H8.E7 disclosed above, does not compete with FLT3L for binding FLT3.

In certain embodiments, the second antigen-binding site competes for binding to FLT3 (e.g., human FLT3, cynomolgus FLT3) with an antigen-binding site described above. In certain embodiments, the second antigen-binding site competes with an antigen-binding site related to 1A2.A3 disclosed above for binding to FLT3. In one embodiment, the second antigen-binding site competes with 1A2.A3 for binding to FLT3. In certain embodiments, the second antigen-binding site of the present invention competes with an antigen-binding site related to 4A4.A3 disclosed above for binding to FLT3. In one embodiment, the second antigen-binding site competes with 4A4.A3 for binding to FLT3. In certain embodiments, the second antigen-binding site of the present invention competes with an antigen-binding site related to 4H2.E3 disclosed above for binding to FLT3. In one embodiment, the second antigen-binding site competes with 4H2.E3 for binding to FLT3. In certain embodiments, the second antigen-binding site of the present invention competes with an antigen-binding site related to 11F4.B9 disclosed above for binding to FLT3. In one embodiment, the second antigen-binding site competes with 11F4.B9 for binding to FLT3.

### Fc Domain

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction. Accordingly, in certain embodiment, the antibody Fc domain or the portion thereof comprises a hinge and a CH2 domain.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as a human IgG1 constant region, an IgG2 constant region, IgG3 constant region, or IgG4 constant region. In one embodiment, the antibody Fc domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence DKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIE KTISKAKGQPREPQVYTLPPSRDELTKNQVSLTCLVKGFYPSDIAVEWESNGQPENN YKTTPPVLDSDGSFFLYSKLTVDKSRWQQGNVFSC SVMHEALHNHYTQKSLSLSPG (SEQ ID NO:136). In some other embodiments, the amino acid sequence of the constant region is at least 90% identical to an antibody constant region from another mammal, such as rabbit, dog, cat, mouse, or horse.

In some embodiments, the antibody constant domain linked to the scFv or the Fab fragment is able to bind to CD16. In some embodiments, the protein incorporates a portion of an antibody Fc domain (for example, a portion of an antibody Fc domain sufficient to bind CD16), wherein the antibody Fc domain comprises a hinge and a CH2 domain (for example, a hinge and a CH2 domain of a human IgG1 antibody), and/or amino acid sequences at least 90% identical to amino acid sequence 234-332 of a human IgG antibody.

One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, K409R, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Alternatively, amino acid substitutions could be selected from the following sets of substitutions shown in Table 3.

TABLE 3

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 4.

TABLE 4

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following sets of substitutions shown in Table 5.

TABLE 5

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 6.

TABLE 6

| First Polypeptide | Second Polypeptide |
|---|---|
| L351Y, D399R, D399K, | T366V, T366I, T366L, |
| S400K, S400R, Y407A, | T366M, N390D, N390E, |
| Y407I, Y407V | K392L, K392M, K392V, |
| | K392F K392D, K392E, |
| | K409F, K409W, T411D and |
| | T411E |

Alternatively, at least one amino acid substitution could be selected from the following sets of substitutions in Table 7, where the position(s) indicated in the First Polypeptide column is replaced by any known negatively-charged amino acid, and the position(s) indicated in the Second Polypeptide Column is replaced by any known positively-charged amino acid.

TABLE 7

| First Polypeptide | Second Polypeptide |
|---|---|
| K392, K370, K409, or K439 | D399, E356, or E357 |

Alternatively, at least one amino acid substitution could be selected from the following set in Table 8, where the position(s) indicated in the First Polypeptide column is replaced by any known positively-charged amino acid, and the position(s) indicated in the Second Polypeptide Column is replaced by any known negatively-charged amino acid.

TABLE 8

| First Polypeptide | Second Polypeptide |
|---|---|
| D399, E356, or E357 | K409, K439, K370, or K392 |

Alternatively, amino acid substitutions could be selected from the following sets in Table 9.

TABLE 9

| First Polypeptide | Second Polypeptide |
|---|---|
| T350V, L351Y, F405A, and | T350V, T366L, K392L, and |
| Y407V | T394W |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of E357, K360, Q362, 5364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, 5364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of L351, D399, 5400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, 5400 and Y407.

In some embodiments, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

In some embodiments, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

In some embodiments, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In some embodiments, the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V and F405T substitutions.

In some embodiments, the amino acid sequence of one polypeptide chain of the antibody constant region differs from the amino acid sequence of an IgG1 constant region by Q347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

### Exemplary Multi-Specific Binding Proteins

Listed below are examples of TriNKETs comprising an antigen-binding site that binds FLT3 and an antigen-binding site that binds NKG2D each linked to an antibody constant region, wherein the antibody constant regions include mutations that enable heterodimerization of two Fc chains. The CDR sequences under Chothia are underlined. F3-GB102 is in the F3 format, i.e., the antigen-binding site that binds FLT3 is an Fab, and the antigen-binding site that binds NKG2D is an scFv. The other TriNKETs are in the F3′ format, i.e., the antigen-binding site that binds FLT3 is an scFv and the antigen-binding site that binds NKG2D is an Fab. In each TriNKET, the scFv comprises substitution of Cys for the amino acid residues at position 100 of VL and position 44 of VH, thereby facilitating formation of a disulfide bridge between the VH and VL of the scFv.

The VH and VL of the scFv can be connected via a linker, e.g., a peptide linker. In certain embodiments, the peptide linker is a flexible linker. Regarding the amino acid composition of the linker, peptides are selected with properties that confer flexibility, do not interfere with the structure and function of the other domains of the proteins of the present invention, and resist cleavage from proteases. For example, glycine and serine residues generally provide protease resistance. In certain embodiments, the VL is linked N-terminal or C-terminal to the VH via a (GlyGlyGlyGlySer)₄ ((G₄S)₄) linker (SEQ ID NO:137).

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, the linker comprises or consists of a (GS)ₙ (SEQ ID NO:290), (GGS)ₙ (SEQ ID NO:291), (GGGS)ₙ (SEQ ID NO:292), (GGSG)ₙ (SEQ ID NO:293), (GGSGG)ₙ (SEQ ID NO:294), and (GGGGS)ₙ (SEQ ID NO:295) sequence, wherein n is 1, 2, 3, 4, 5, 6, 7, 8, 9, 10, 11, 12, 13, 14, 15, 16, 17, 18, 19, or 20. In certain embodiments, the linker comprises or consists of an amino acid sequence selected from SEQ ID NO:137, SEQ ID NO:201, SEQ ID NO:202, SEQ ID NO: 103, SEQ ID NO:104, SEQ ID NO:83, SEQ ID NO:84, SEQ ID NO:150, SEQ ID NO:152, and SEQ ID NO:154, as listed in Table 10.

TABLE 10

| SEQ ID | Amino Acid Sequence |
|---|---|
| SEQ ID | GSGSGSGSGSGSGSGSGSGS |
| NO: 296 | |
| SEQ ID | GGSGGSGGSGGSGGSGGSGGSGGSGGSGG |
| NO: 297 | S |
| SEQ ID | GGGSGGGSGGGSGGGSGGGSGGGSGGGSG |
| NO: 298 | GGSGGGSGGGS |
| SEQ ID | GGSGGGSGGGSGGGSGGGSGGGSGGGSGG |
| NO: 299 | GSGGGSGGGSG |
| SEQ ID | GGSGGGGSGGGGSGGGGSGGGGSGGGGSG |
| NO: 300 | GGGSGGGGSGGGGSGGGGSGG |
| SEQ ID | GGGGSGGGGSGGGGSGGGGSGGGGSGGGG |
| NO: 301 | SGGGGSGGGGSGGGGSGGGGS |
| SEQ ID | GGGGSGGGGSGGGGSGGGGS |
| NO: 137 | |
| SEQ ID | GGGGSGGGGSGGGGS |
| NO: 302 | |
| SEQ ID | GGGGSGGGGSGGGGSGGGGSGGGGSGGGG |
| NO: 303 | SGGGGSGGGGSGGGGSGGGGSGGGGSGGG |
| | GSGGGGSGGGGSGGGGSGGGGSGGGGSGG |
| | GGSGGGGSGGGGS |
| SEQ ID | GGSGGGGSGGGGSGGGGSGGGGSGGGGSG |
| NO: 304 | GGGSGGGGSGGGGSGGGGSGGGGSGGGGS |
| | GGGGSGGGGSGGGGSGGGGSGGGGSGGGG |
| | SGGGGSGGGGSGG |

In F3-GB102, the NKG2D-binding scFv is linked to the N-terminus of an Fc via an Ala-Ser linker. In the F3′-TriNKETs, the FLT3-binding scFv is linked to the N-terminus of an Fc via a Gly-Ser linker. The Ala-Ser or Gly-Ser linker is included at the elbow hinge region sequence to balance between flexibility and optimal geometry. In certain embodiments, an additional sequence Thr-Lys-Gly can be added N-terminal or C-terminal to the Ala-Ser or Gly-Ser sequence at the hinge.

As used herein to describe these exemplary TriNKETs, Fc includes an antibody hinge, CH2, and CH3. In each exemplary TriNKET, the Fc domain linked to an scFv comprises the mutations of Q347R, D399V, and F405T, and the Fc domain linked to an Fab comprises matching mutations K360E and K409W for forming a heterodimer. The Fc domain linked to the scFv further includes an S354C substitution in the CH3 domain, which forms a disulfide bond with a Y349C substitution on the Fc linked to the Fab. These substitutions are bold in the sequences described in this subsection.

For example, a TriNKET of the present disclosure is F3′-GB102. F3′-GB102 includes (a) an FLT3-binding scFv sequence derived from GB102 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB102 includes three polypeptides as set forth below.

GB102-VL-VH-Fc
(SEQ ID NO: 277)
DIVMTQSPDSLAVSLGERATINCRASESVDTYGSSFVHWYQQKPGQPPKL
LIYLASNLESGVPDRFSGSGSGTDFTLTISSLQAEDVAVYYCQQNNEEPW
TFGCGTKVEIKGGGGSGGGGSGGGGSGGGGSQVQLVQSGAEVKKPGASVK
VSCKASGYTFTRYVMHWVRQAPGQCLEWMGFINPYNDDTKYNEKFKGRVT
ITRDTSASTAYMELSSLRSEDTAVYYCARwrqlgsldsWGQGTTVTVSSG
SDKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKEY
KCKVSNKALPAPIEKTISKAKGQPREPRVYTLPPCRDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG
A49MI-VH-CH1-Fc
(SEQ ID NO: 278)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PIGAAAGWEDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVCTLPPSRDELTENQVSLTCLVKGFYPSDIAVEWESNGQPENNYKTTP
PVLDSDGSFFLYSWLTVDKSRWQQGNVFSCSVMHEALHNHYTQKSLSLSP
G
A49MI-VL-CL
(SEQ ID NO: 279)
DIQMTQSPSSVSASVGDRVTITCRASQGISSWLAWYQQKPGKAPKLLIYA
ASSLQSGVPSRFSGSGSGTDFTLTISSLQPEDFATYYCQQGVSFPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

GB102-VL-VH-Fc represents the full sequence of an FLT3-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv (SEQ ID NO:40) includes a heavy chain variable domain of GB102 connected to the C-terminus of a light chain variable domain of GB102 via a (G₄S)₄ linker. The heavy and the light variable domains of the scFv are also connected through a disulfide bridge between C100 of VL and C44 of VH, as a result of R100C and G44C substitutions in the VL and VH, respectively.

A49MI-VH-CH1-Fc represents the heavy chain portion of the Fab fragment, which comprises a heavy chain variable domain (SEQ ID NO:254) of NKG2D-binding A49MI and a CH1 domain, connected to an Fc domain. The Fc domain in A49MI-VH-CH1-Fc includes a Y349C substitution in the CH3 domain, which forms a disulfide bond with an S354C substitution on the Fc in GB102-VL-VH-Fc. In A49MI-VH-CH1-Fc, the Fc domain also includes K360E and K409W substitutions for heterodimerization with the Fc in GB102-VL-VH-Fc.

A49MI-VL-CL represents the light chain portion of the Fab fragment comprising a light chain variable domain of NKG2D-binding A49MI (SEQ ID NO:239) and a light chain constant domain.

Another TriNKET of the present disclosure is F3-GB102. F3-GB102 includes (a) an NKG2D-binding scFv sequence derived from A49 linked to an Fc domain and (b) an FLT3-binding Fab fragment derived from GB102 including a heavy chain portion comprising a heavy chain variable domain and a CH1 domain, and a light chain portion comprising a light chain variable domain and a light chain constant domain, wherein the CH1 domain is connected to the Fc domain. F3-GB102 includes three polypeptides as set forth below.

A49-VL-VH-Fc
(SEQ ID NO: 280)
DIQMTQSPSSVSASVGDRVTITCRASQGISSWLAWYQQKPGKAPKLLIYA
ASSLQSGVPSRFSGSGSGTDFTLTISSLQPEDFATYYCQQGVSFPRTFGC
GTKVEIKGGGGSGGGGSGGGGSGGGGSEVQLVESGGGLVKPGGSLRLSCA
ASgftfssySMNWVRQAPGKCLEWVSSISSSSSYIYYADSVKGRFTISRD
NAKNSLYLQMNSLRAEDTAVYYCARGAPMGAAAGWFDPWGQGTLVTVSSA
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPRVYTLPPCRDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG
GB102-VH-CH1-Fc
(SEQ ID NO: 281)
QVQLVQSGAEVKKPGASVKVSCKASGYTFTRYVMHWVRQAPGQGLEWMGF
INPYNDDTKYNEKFKGRVTITRDTSASTAYMELSSLRSEDTAVYYCARWR
QLGSLDSWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVC
TLPPSRDELTENQVSLTCLVKGFYPSDIAVEWESNGQPENNYKTTPPVLD
SDGSFFLYSWLTVDKSRWQQGNVFSCSVMHEALHNHYTQKSLSLSPG
GB102-VL-CL
(SEQ ID NO: 282)
DIVMTQSPDSLAVSLGERATINCRASESVDTYGSSFVHWYQQKPGQPPKL
LIYLASNLESGVPDRFSGSGSGTDFTLTISSLQAEDVAVYYCQQNNEEPW
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

A49-VL-VH-Fc represents the full sequence of an NKG2D-binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in GB102-VH-CH1-Fc as described below. The scFv (SEQ ID NO:246) includes a heavy chain variable domain of A49 connected to the C-terminus of a light chain variable domain of A49 via a (G₄S)₄ linker. The heavy and the light variable domains of the scFv are also connected through a disulfide bridge between C100 of VL and C44 of VH, as a result of Q100C and G44C substitutions in the VL and VH, respectively.

GB102-VH-CH1-Fc represents the heavy chain portion of the Fab fragment, which comprises a heavy chain variable domain (SEQ ID NO:37) of FLT3-binding GB102 and a CH1 domain, connected to an Fc domain. The Fc domain in GB102-VH-CH1-Fc includes a Y349C substitution in the CH3 domain, which forms a disulfide bond with an S354C substitution on the Fc in A49-VL-VH-Fc. In GB102-VH-CH1-Fc, the Fc domain also includes K360E and K409W substitutions for heterodimerization with the Fc in A49-VL-VH-Fc.

GB102-VL-CL represents the light chain portion of the Fab fragment comprising a light chain variable domain of FLT3-binding GB102 (SEQ ID NO:38) and a light chain constant domain.

Another TriNKET of the present disclosure is F3′-1553. F3′-1553 includes (a) an FLT3-binding scFv sequence derived from mAb 1553 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-1553 includes three polypeptides: 1553-VH-VL-Fc, A49MI-VH-CH1-Fc, A49MI-VL-CL. A49MI-VH-CH1-Fc and A49MI-VL-CL are described above in the context of F3′-GB102. The polypeptide of 1553-VH-VL-Fc is set forth below.

1553-VH-VL-Fc
(SEQ ID NO: 283)
QVQLVQSGAEVKKPGASVKVSCKVSGYTFTSYWINWVRQAPGKCLEWMGN
IYPGSSIINYNENFKNRVTMTEDTSTDTAYMELSSLRSEDTAVYYCARRV
VYLYFDYWGQGTLVTVSSGGGGSGGGGSGGGGSGGGGSEIVLTQSPATLS
LSPGERATLSCSASSSVSYMHWYQQKPGQAPRLLIYdtsklasGIPARFS
GSGSGTDFTLTISSLEPEDFAVYYCQQWTSKSPTFGCGTKVEIKGSDKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPRVYTLPPCRDELTKNQVSLTCLVKGFYP
SDIAVEWESNGQPENNYKTTPPVLVSDGSFTLYSKLTVDKSRWQQGNVFS
CSVMHEALHNHYTQKSLSLSPG

1553-VH-VL-Fc represents the full sequence of an FLT3-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv (SEQ ID NO:74) includes a heavy chain variable domain of 1553 connected to the N-terminus of a light chain variable domain of 1553 via a (G₄S)₄ linker. The heavy and the light variable domains of the scFv are also connected through a disulfide bridge between C100 of VL and C44 of VH, as a result of G100C and G44C substitutions in the VL and VH, respectively.

Another TriNKET of the present disclosure is F3′-1689. F3′-1689 includes (a) an FLT3-binding scFv sequence derived from mAb 1689 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-1689 includes three polypeptides: 1689-VH-VL-Fc, A49MI-VH-CH1-Fc, A49MI-VL-CL. A49MI-VH-CH1-Fc and A49MI-VL-CL are described above in the context of F3′-GB102. The polypeptide of 1689-VH-VL-Fc is set forth below.

1689-VH-VL-Fc
(SEQ ID NO: 284)
QVQLVQSGAEVKKPGASVKVSCKVSGYTFPYYWINWVRQAPGKCLEWMGN
IYPGSSIINYNENFKNRVTMTEDTSTDTAYMELSSLRSEDTAVYYCARRN
VYLTFDYWGQGTLVTVSSGGGGSGGGGSGGGGSGGGGSEIVLTQSPATLS
LSPGERATLSCSASSSVSYIHWYQQKPGQAPRLLIYDTSKLASGIPARFS
GSGSGTDFTLTISSLEPEDFAVYYCqqwtsksptFGCGTKVEIKGSDKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPRVYTLPPCRDELTKNQVSLTCLVKGFYP
SDIAVEWESNGQPENNYKTTPPVLVSDGSFTLYSKLTVDKSRWQQGNVFS
CSVMHEALHNHYTQKSLSLSPG

1689-VH-VL-Fc represents the full sequence of an FLT3-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv (SEQ ID NO:81) comprises a set of mutations relative to the scFv in 1553-VH-VL-Fc that potentially increases binding affinity to FLT3.

Another TriNKET of the present disclosure is F3′-GB102 M34I. F3′-GB102 M34I includes (a) an FLT3-binding scFv sequence derived from GB102 M34I linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB102 M34I includes three polypeptides: GB102 M34I-VL-VH-Fc, A49MI-VH-CH1-Fc, A49MI-VL-CL. A49MI-VH-CH1-Fc and A49MI-VL-CL are described above in the context of F3′-GB102. The polypeptide of GB102_M34I-VL-VH-Fc is set forth below.

GB102 M34I-VL-VH-Fc
(SEQ ID NO: 285)
DIVMTQSPDSLAVSLGERATINCRASESVDTYGSSFVHWYQQKPGQPPKL
LIYLASNLESGVPDRFSGSGSGTDFTLTISSLQAEDVAVYYCQQNNEEPW
TFGCGTKVEIKGGGGSGGGGSGGGGSGGGGSQVQLVQSGAEVKKPGASVK
VSCKASGYTFTRYVIHWVRQAPGQCLEWMGFINPYNDDTKYNEKFKGRVT
ITRDTSASTAYMELSSLRSEDTAVYYCARWRQLGSLDSWGQGTTVTVSSG
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPRVYTLPPCRDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG

GB102 M34I-VL-VH-Fc represents the full sequence of an FLT3-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv (SEQ ID NO:48) comprises an M34I substitution relative to the VH in GB102-VL-VH-Fc to remove a putative sequence liability.

Another TriNKET of the present disclosure is F3′-GB102 D101E. F3′-GB102_D101E includes (a) an FLT3-binding scFv sequence derived from GB102 D101E linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB102 D101E includes three polypeptides: GB102 D101E-VL-VH-Fc, A49MI-VH-CH1-Fc, A49MI-VL-CL. A49MI-VH-CH1-Fc and A49MI-VL-CL are described above in the context of F3′-GB102. The polypeptide of GB102 D101E-VL-VH-Fc is set forth below.

GB102 D101E-VL-VH-Fc
(SEQ ID NO: 286)
DIVMTQSPDSLAVSLGERATINCRASESVDTYGSSFVHWYQQKPGQPPKL
LIYLASNLESGVPDRFSGSGSGTDFTLTISSLQAEDVAVYYCQQNNEEPW
TFGCGTKVEIKGGGGSGGGGSGGGGSGGGGSQVQLVQSGAEVKKPGASVK
VSCKASGYTFTRYVMHWVRQAPGQCLEWMGFINPYNDDTKYNEKFKGRVT
ITRDTSASTAYMELSSLRSEDTAVYYCARWRQLGSLESWGQGTTVTVSSG
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPRVYTLPPCRDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG

GB102_D101E-VL-VH-Fc represents the full sequence of an FLT3-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv (SEQ ID NO:44) comprises a D101E substitution relative to the VH in GB102-VL-VH-Fc to remove a putative sequence liability.

Another TriNKET of the present disclosure is F3′-GB102_M34I_D101E. F3′-GB102_M34I_D101E includes (a) an FLT3-binding scFv sequence derived from GB102 M34I/D101E linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB102_M34I_D101E includes three polypeptides: GB102_M34I_D101E-VL-VH-Fc, A49MI-VH-CH1-Fc, A49MI-VL-CL. A49MI-VH-CH1-Fc and A49MI-VL-CL are described above in the context of F3′-GB102. The polypeptide of GB102_M34I_D101E-VL-VH-Fc is set forth below.

GB102 M34I D101E-VL-VH-Fc
(SEQ ID NO: 287)
DIVMTQSPDSLAVSLGERATINCRASESVDTYGSSFVHWYQQKPGQPPKL
LIYLASNLESGVPDRFSGSGSGTDFTLTISSLQAEDVAVYYCQQNNEEPW
TFGCGTKVEIKGGGGSGGGGSGGGGSGGGGSQVQLVQSGAEVKKPGASVK
VSCKASGYTFTRYVIHWVRQAPGQCLEWMGFINPYNDDTKYNEKFKGRVT
ITRDTSASTAYMELSSLRSEDTAVYYCARWRQLGSLESWGQGTTVTVSSG
SDKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKEY
KCKVSNKALPAPIEKTISKAKGQPREPRVYTLPPCRDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG

GB102_M34I_D101E-VL-VH-Fc represents the full sequence of an FLT3-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv (SEQ ID NO:52) comprises M34I and D101E substitutions relative to the VH in GB102-VL-VH-Fc to remove putative sequence liabilities.

Another TriNKET of the present disclosure is F3′-GB99. F3′-GB99 includes (a) an FLT3-binding scFv sequence derived from GB99 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB99 includes three polypeptides: GB99-VL-VH-Fc, A49MI-VH-CH1-Fc, A49MI-VL-CL. A49MI-VH-CH1-Fc and A49MI-VL-CL are described above in the context of F3′-GB102. The polypeptide of GB99-VL-VH-Fc is set forth below.

GB99-VL-VH-Fc
(SEQ ID NO: 288)
DIVMTQSPASLAVSLGERATINCRASESVDTYGSSFVHWYQQKPGQPPKL
LIYLASNLESGVPDRFSGSGSGTDFTLTISSLQAEDAATYYCQQNNEEPW
TFGCGTKVEIKGGGGSGGGGSGGGGSGGGGSQVQLVQSGAEVKKPGASVK
VSCKASGYTFTRYVMHWVRQAPGQCLEWMGFINPYNDDTKYNEKFKGRVT
ITSDTSASTAYMELSSLRSEDTAVYHCARWRQLGSLDSWGQGTTVTVSSG
SDKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKEY
KCKVSNKALPAPIEKTISKAKGQPREPRVYTLPPCRDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG

GB99-VL-VH-Fc represents the full sequence of an FLT3-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv (SEQ ID NO:28) comprises a set of back mutations in the framework regions relative to the scFv in GB102-VL-VH-Fc to potentially improve antibody structure and activity.

Another TriNKET of the present disclosure is F3′-GB89. F3′-GB89 includes (a) an FLT3-binding scFv sequence derived from GB89 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB89 includes three polypeptides: GB89-VH-VL-Fc, A49MI-VH-CH1-Fc, A49MI-VL-CL. A49MI-VH-CH1-Fc and A49MI-VL-CL are described above in the context of F3′-GB102. The polypeptide of GB89-VH-VL-Fc is set forth below.

GB89-VH-VL-Fc
(SEQ ID NO: 289)
QVQLVQSGAEVKKPGASVKVSCKASGYTFTRYVMHWVRQAPGQCLEWMGF
INPYNDDTKYNEKFKGRVTITSDTSASTAYMELSSLRSEDTAVYYCARWR
QLGSLDSWGQGTTVTVSSGGGGSGGGGSGGGGSGGGGSDIVMTQSPASLA
VSLGERATINCRASESVDTYGSSFVHWYQQKPGQPPKLLIYLASNLESGV
PDRFSGSGSRTDFTLTISSLQAEDAATYYCQQNNEEPWTFGCGTKVEIKG
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPRVYTLPPCRDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG

GB89-VH-VL-Fc represents the full sequence of an FLT3-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv (SEQ ID NO:19) includes a heavy chain variable domain of GB89 connected to the N-terminus of a light chain variable domain of GB89 via a (G₄S)₄ linker. The heavy and the light variable domains of the scFv are also connected through a disulfide bridge between C100 of VL and C44 of VH, as a result of R100C and G44C substitutions in the VL and VH, respectively. The scFv also comprises a set of back mutations in the framework regions relative to the VH and VL of GB102-VL-VH-Fc to potentially improve antibody structure and activity.

In a certain embodiment, a TriNKET of the present disclosure is identical to one of the exemplary TriNKETs described above that includes the EW-RVT Fc mutations, except that the Fc domain linked to the NKG2D-binding Fab fragment comprises the substitutions of Q347R, D399V, and F405T, and the Fc domain linked to the HER2-binding scFv comprises matching substitutions K360E and K409W for forming a heterodimer. In certain embodiments, a TriNKET of the present disclosure is identical to one of the exemplary TriNKETs described above that includes the KiH Fc mutations, except that the Fc domain linked to the NKG2D-binding Fab fragment comprises the “hole” substitutions of T366S, L368A, and Y407V, and the Fc domain linked to the HER2-binding scFv comprises the “knob” substitution of T366W for forming a heterodimer.

In certain embodiments, a TriNKET of the present disclosure is identical to one of the exemplary TriNKETs described above, except that the Fc domain linked to the NKG2D-binding Fab fragment includes an S354C substitution in the CH3 domain, and the Fc domain linked to the HER2-binding scFv includes a matching Y349C substitution in the CH3 domain for forming a disulfide bond.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that comprise an Fc domain at its C-terminus. Accordingly, in some embodiments where the C-terminal residue of an amino acid sequence of a polypeptide (e.g., an Fc domain sequence) is K, a corresponding amino acid sequence with the K removed is also contemplated herein.

The multi-specific proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

To achieve the highest yield of the multi-specific protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multi-specific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of the Multi-Specific Proteins

The multi-specific proteins described herein include an NKG2D-binding site, a FLT3-binding site that binds FLT3, and an antibody Fc domain or a portion thereof sufficient to bind CD16, or an antigen-binding site that binds CD16. In some embodiments, the multi-specific proteins contains an additional antigen-binding site that binds to FLT3, as exemplified in the F4-TriNKET format.

In some embodiments, the multi-specific proteins display similar thermal stability to the corresponding monoclonal antibody, i.e., a monoclonal antibody containing the same FLT3-binding site as the one incorporated in the multi-specific proteins.

In some embodiments, the multi-specific proteins simultaneously bind to cells expressing NKG2D and/or CD16, such as NK cells, and cells expressing FLT3, such as certain tumor cells. Binding of the multi-specific proteins to NK cells can enhance the activity of the NK cells toward destruction of the FLT3-expressing tumor cells.

In some embodiments, the multi-specific proteins bind to FLT3 with a similar affinity to the corresponding the anti-FLT3 monoclonal antibody (i.e., a monoclonal antibody containing the same FLT3-binding site as the one incorporated in the multi-specific proteins). In some embodiments, the multi-specific proteins are more effective in killing the tumor cells expressing FLT3 than the corresponding monoclonal antibodies.

In certain embodiments, the multi-specific proteins described herein, which include a binding site for FLT3, activate primary human NK cells when co-culturing with cells expressing FLT3. NK cell activation is marked by the increase in CD107a degranulation and IFN-γ cytokine production. Furthermore, compared to a corresponding anti-FLT3 monoclonal antibody, the multi-specific proteins can show superior activation of human NK cells in the presence of cells expressing FLT3.

In some embodiments, the multi-specific proteins described herein, which include a binding site for FLT3, enhance the activity of rested and IL-2-activated human NK cells when co-culturing with cells expressing FLT3.

In some embodiments, compared to the corresponding monoclonal antibody that binds to FLT3, the multi-specific proteins offer an advantage in targeting tumor cells that express medium and low levels of FLT3.

In some embodiments, the bivalent F4 format of the TriNKETs (i.e., TriNKETs include an additional antigen-binding site that binds to FLT3) improve the avidity with which the TriNKETs binds to FLT3, which in effect stabilize expression and maintenance of high levels of FLT3 on the surface of the tumor cells. In some embodiments, the F4-TriNKETs mediate more potent killing of tumor cells than the corresponding F3-TriNKETs or F3′-TriNKETs.

### III. Therapeutic Applications

The invention provides methods for treating autoimmune disease or cancer using a multi-specific binding protein described herein and/or a pharmaceutical composition described herein. The methods may be used to treat a variety of cancers expressing FLT3.

The therapeutic method can be characterized according to the cancer to be treated. For example, in certain embodiments, the cancer is a hematologic malignancy or leukemia. In certain embodiments, the cancer is acute myeloid leukemia (AML), acute lymphoblastic leukemia (ALL), myelodysplasia, myelodysplastic syndromes, acute T-lymphoblastic leukemia, or acute promyelocytic leukemia, chronic myelomonocytic leukemia, or myeloid blast crisis of chronic myeloid leukemia.

Other exemplary cancers to be treated by the FLT3 targeting multi-specific binding proteins include breast cancer, ovarian cancer, esophageal cancer, bladder or gastric cancer, salivary duct carcinoma, salivary duct carcinomas, adenocarcinoma of the lung or aggressive forms of uterine cancer, such as uterine serous endometrial carcinoma. In some other embodiments, the cancer is brain cancer, breast cancer, cervical cancer, colon cancer, colorectal cancer, endometrial cancer, esophageal cancer, leukemia, lung cancer, liver cancer, melanoma, ovarian cancer, pancreatic cancer, rectal cancer, renal cancer, stomach cancer, testicular cancer, or uterine cancer. In yet other embodiments, the cancer is a squamous cell carcinoma, adenocarcinoma, small cell carcinoma, melanoma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well-differentiated carcinoma, or Wilms tumor.

In some other embodiments, the cancer to be treated is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

### IV. Combination Therapy

Another aspect of the invention provides for combination therapy. A multi-specific binding protein described herein can be used in combination with additional therapeutic agents to treat autoimmune disease or to treat cancer.

Exemplary therapeutic agents that may be used as part of a combination therapy in treating autoimmune inflammatory diseases are described in Li et al. (2017) Front. Pharmacol., 8:460, and include, for example, non-steroidal anti-inflammatory drugs (NSAIDs) (e.g., COX-2 inhibitors), glucocorticoids (e.g., prednisone/prednisolone, methylprednisolone, and the fluorinated glucocorticoids such as dexamethasone and betamethasone), disease-modifying antirheumatic drugs (DMARDs) (e.g., methotrexate, leflunomide, gold compounds, sulfasalazine, azathioprine, cyclophosphamide, antimalarials, D-penicillamine, and cyclosporine), anti-TNF biologics (e.g., infliximab, etanercept, adalimumab, golimumab, Certolizumab pegol, and their biosimilars), and other biologics targeting CTLA-4 (e.g., abatacept), IL-6 receptor (e.g., tocilizumab), IL-1 (e.g., anakinra), Th1 immune responses (IL-12/IL-23) (e.g., ustekinumab), Th17 immune responses (IL-17) (e.g., secukinumab) and CD20 (e.g., rituximab).

Exemplary therapeutic agents that may be used as part of a combination therapy in treating cancer include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-γ), colony stimulating factor-1, colony stimulating factor-2, denileukin diftitox, interleukin-2, luteinizing hormone releasing factor and variations of the aforementioned agents that may exhibit differential binding to its cognate receptor, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze-dried formulation from 12, 27, or 45 vials are combined to obtained a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

The protein could exist in a liquid aqueous pharmaceutical formulation including a therapeutically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86 mg/mL), and about 6.2 mg/mL of sodium chloride (e.g., 6.165 mg/mL). In certain embodiments, the buffer system includes about 1 to about 1.5 mg/mL of citric acid, about 0.25 to about 0.5 mg/mL of sodium citrate, about 1.25 to about 1.75 mg/mL of disodium phosphate dihydrate, about 0.7 to about 1.1 mg/mL of sodium dihydrogen phosphate dihydrate, and about 6.0 to about 6.4 mg/mL of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10 to about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the protein product solution in order to allow an extractable volume of 60 mL. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation of the present disclosure may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

The protein of the present disclosure could exist in a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 ng/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### Example 1. Characterization of Supernatants of Selected Hybridoma Clones

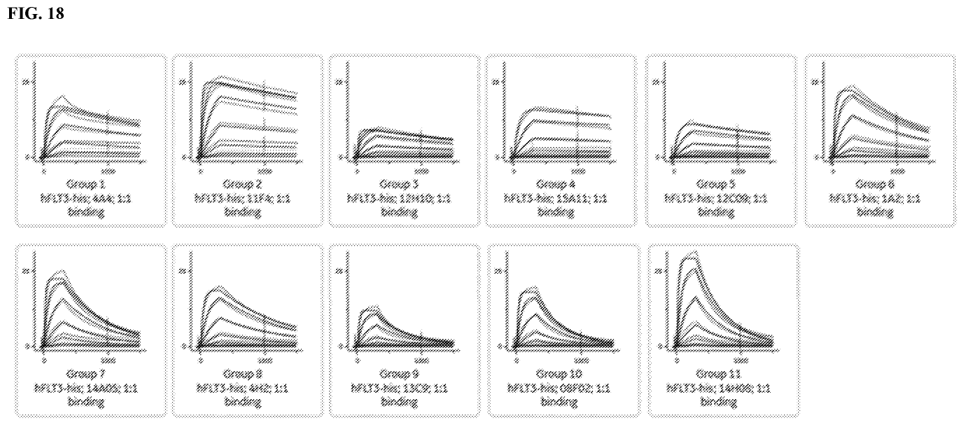

FLT3-specific antibodies were generated by immunizing mice with hFLT3-His fusion protein. Supernatants of 228 hybridomas were assessed for FLT3 binding by enzyme-linked immunosorbent assay (ELISA), and 96 hybridomas bound noncovalently to hFLT3-His protein. Eleven clones were selected based on preliminary Bio-layer Interferometry (BLI) binding affinity estimations, binding to human and cynomolgus monkey cell expressing FLT3, and diversity of epitopes. The ability of these 11 clones to bind hFLT3-His was further analyzed by high resolution surface plasmon resonance (SPR). The experiment was performed at 37° C. to mimic physiological temperature using a Biacore 8K instrument. Biacore sensorgrams and kinetic parameters are presented in Table 12 and raw data and fits are shown in FIG. 18. Seven out of eleven hybridomas bound with K_{D} less than 10 nM, and five display slow dissociation rate constant (k_{d}<5×10⁻⁴ s⁻¹).

Binning of hybridoma fusions with reference mAbs was performed by BLI using OctetRed384 (ForteBio). Briefly, hybridoma supernatants were loaded onto anti-mouse IgG capture sensor tips for 15 minutes and equilibrated for 5 minutes in PB SF. Sensors were dipped into 200 nM hFLT3-His and allowed to associate for 180 seconds followed by dipping into 100 nM control IgGs or 200 nM FTL3-ligand solution. The increase in response units indicated the hybridoma was a non-competitor to the reference mAb, while no increase in signal indicated that hybridoma did compete with the reference mAb. FL23 (Amgen) and FL39 (Amgen) bind to Domain 1. EB10 (ImClone), a known FLT3-ligand blocker, binds to Domain 3. FL61 (Amgen) also binds to domain 3, but is not a FLT3-ligand blocker. 4G8 (Synimmune) binds to Domain 4. NC7 (Imclone) binds to Domain 5. The VH and VL sequences of these reference antibodies are provided in Table 11.

TABLE 11
Reference antibodies

| a-FLT3 mAb | VH | VL |
|---|---|---|
| 4G8 | QVQLQQPGAELVKPGASLKLSCK | DIVLTQSPATLSVTPGDSVSLS |
| (Synimmune), | SSGYTFTSYWMHWVRQRPGHGLE | CRASQSISNNLHWYQQKSHESP |
| disclosed in U.S. | WIGEIDPSDSYKDYNQKFKDKAT | RLLIKYASQSISGIPSRFSGSG |
| Application | LTVDRSSNTAYMHLSSLTSDDSA | SGTDFTLSINSVETEDFGVYFC |
| Publication No. | VYYCARAITTTPFDFWGQGTTLT | QQSNTWPYTFGGGTKLEIK |
| 2015/0119555A1 | VSS (SEQ ID NO: 306) | (SEQ ID NO: 307) |
| EB10 | EVQLVQSGAEVKKPGASVKVSCK | DVVMTQSPLSLPVTPGEPASIS |
| (ImClone/Lilly), | ASGYTFTSYYMHWVRQAPGQGLE | CRSSQSLLHSNGNNYLDWYLQK |
| disclosed in U.S. | WMGIINPSGGSTSYAQKFQGRVT | PGQSPQLLIYLGSNRASGVPDR |
| Application | MTRDTSTSTVYMELSSLRSEDTA | FSGSGSDTDFTLQISRVEAEDV |
| Publication No. | VYYCARGVGAHDAFDIWGQGTTV | GVYYCMQGTHPAISFGQGTRLE |
| 2011/0008355A1 | TVSS (SEQ ID NO: 308) | IK (SEQ ID NO: 309) |
| NC7 | EVQLVQSGAEVKKPGSSVKVSCK | DIQMTQSPSSLSASVGDRVTIT |
| (Imclone/Lilly), | ASGGTFSSYAISWVRQAPGQGLE | CRASQSISSYLNWYQQKPGKAP |
| disclosed in U.S. | WMGGIIPIFGTANYAQKFQGRVT | KLLIYAASSLQSGVPSRFSGSG |
| Application | ITADKSTSTAYMELSSLRSEDTA | SGTDFTLTISSLQPEDLATYYC |
| Publication No. | VYYCATFALFGFREQAFDIWGQG | QQSYSTPFTFGPGTKVDIK |
| 2011/0008355A1 | TTVTVSS (SEQ ID NO: | (SEQ ID NO: 311) |
| | 310) | |
| FL23 (Amgen), | QVTLKESGPALVKPTETLTLTCT | DIQMTQSPSSLSASVGDRVTIT |
| disclosed in U.S. | VSGFSFRNARMGVSWIRQPPGKA | CRASQDIGYDLGWYQQKPGKAP |
| Application | LEWLAHIFSNDEKSYSTSLKSRL | KRLIYAASTLQSGVPSRFSGSG |
| Publication No. | TISKDTSKSQVVLTLTNMDPVDT | SGTEFTLIISSLQPEDFATYYC |
| 2017/0037149A1 | ATYFCARMPEYSSGWSGAFDIWG | LQHNSFPWTFGQGTKVEIK |
| | QGTMVTVSS (SEQ ID NO: | (SEQ ID NO: 313) |
| | 312) | |
| FL39 (Amgen), | QVTLKESGPTLVKPTETLTLTCT | DIQMTQSPSSLSASVGDRVTIT |
| disclosed in U.S. | LSGFSLNNARMGVSWIRQPPGKC | CRASQGIRNDLGWYQQKPGKAP |
| Application | LEWLAHIFSNDEKSYSTSLKNRL | KRLIYAASTLQSGVPSRFSGSG |
| Publication No. | TISKDSSKTQVVLTMTNVDPVDT | SGTEFTLTISSLQPEDFATYYC |
| 2017/0037149A1 | ATYYCARIVGYGSGWYGFFDYWG | LQHNSYPLTFGCGTKVEIK |
| | QGTLVTVSS (SEQ ID NO: | (SEQ ID NO: 315) |
| | 314) | |
| FL61 (Amgen), | QVQLVESGGGVVQPGRSLRLSCA | DIQMTQSPSSLSASVGDRVTIT |
| disclosed in U.S. | ASGFTFSSYGMHWVRQAPGKGLE | CRASQSISSYLNWYQQKPGKAP |
| Application | WVAVISYDGSNEFYADSVKGRFT | KLLIYAASSLQSGVPSRFSGSG |
| Publication No. | ISRDNSKNTLYLQMNSLRAEDTA | SGTEFTLTISSLQPEDFATYYC |
| 2017/0037149A1 | VYYCARGGEITMVRGVIGYYYYG | LQHNSYPLTFGGGTKVEIK |
| | MDVWGQGTTVTVSS | (SEQ ID NO: 317) |
| | (SEQ ID NO: 316) | |

It was observed that antibodies produced from five of the hybridomas, namely 4A4, 11F4, 1A2, 4H2, and 13C9, did not compete with any of the reference antibodies for binding to hFLT3-His. Cross-reactivity with cynomolgus monkey FLT3 (cFLT3) was evaluated by measuring the binding of the antibodies to isogenic RMA cells expressing cFLT3.

Briefly, RMA cells were transducted with a retroviral vector encoding cFLT3 or human FLT3 (hFLT3). Binding of the α-FLT3 mAbs from crude hybridoma harvests to the hFLT3 or cFLT3 isogenic cell lines, as well as FLT3+ cancer cell lines, was performed as follows. 100,000 RMA, REH or SEM cells were added per well of a 96 well round bottom plate. Cells were spun down and the pellet was gently dissociated by vortexing. 50 μL of Zombie live/dead dye (PBS+1:2000 dye) were added per well and incubated in the dark at room temperature for 20 minutes. Cells were washed with 200 μL of FACS buffer (PBS+2% FBS). 50 μL of hybridoma supernatants were added to the washed cells and the mixtures were incubated for 30 minutes on ice in the dark. Cells were washed once and then 50 μL of anti-mouse Fc-PE secondary reagent (1:200 dilution) were added and incubated for 20 minutes on ice in the dark. Cells were washed and fixed with 50 μL of 4% paraformaldehyde for 15 minutes on ice. Cells were washed again and then resuspended in 200 μL FACS buffer and stored at 4° C. until ready for acquisition. The samples were run on BD FACSCelesta equipped with an HTS (high throughput sampler).

The binding affinities of the hybridoma supernatants to REH cancer cells (ATCC catalog number CRL-8286), a human ALL cell line reported to express FLT3, were also measured. As shown in Table 12, most of the clones displayed binding affinity to cancer cells expressing hFLT3 and cross-reactivity with cFLT3. Cynomolgus monkey FLT3 binding data for 14A5 and 15A11 were not collected.

TABLE 12
Kinetic parameters and affinities of FLT3-His binding
to the antibodies produced from candidate hybridomas

| | | SPR at 37° C. | | | Cell Binding MFI | | |
|---|---|---|---|---|---|---|---|
| Test | Binning | | | K_{D} | RMA- | RMA- | |
| articles | profile | kₐ (1/Ms) | k_{d} (1/s) | (nM) | hFLT3 | cFLT3 | REH |
| 4A4 | unique | 3.38 × 10⁵ | 3.35 × 10⁻⁴ | 1.0 | 1493 | 2002 | 2002 |
| 11F4 | unique | 1.73 × 10⁵ | 1.88 × 10⁻⁴ | 1.1 | 305 | 495 | 495 |
| 12H10 | 4G8 | 1.74 × 10⁵ | 3.43 × 10⁻⁴ | 2.0 | 544 | 696 | 696 |
| 15A11 | EB10 | 4.99 × 10⁵ | 1.17 × 10⁻⁴ | 2.3 | 332 | n/a | n/a |
| 12C9 | FL23 | 5.67 × 10⁴ | 3.11 × 10⁻⁴ | 5.4 | 1020 | 3937 | 664 |
| 1A2 | unique | 1.14 × 10⁵ | 7.49 × 10⁻⁴ | 6.5 | 238 | 461 | 461 |
| 14A5 | FL23 | 1.70 × 10⁵ | 1.49 × 10⁻³ | 8.7 | 1005 | n/a | 2071 |
| 4H2 | unique | 8.05 × 10⁴ | 9.18 × 10⁻⁴ | 11 | 570 | 1017 | 1017 |
| 13C9 | unique | 2.12 × 10⁵ | 3.02 × 10⁻³ | 14 | 546 | 834 | 834 |
| 8F02 | FL23 | 1.67 × 10⁵ | 2.80 × 10⁻³ | 17 | 829 | 2729 | 2271 |
| 14H08 | FL23 | 1.29 × 10⁵ | 2.40 × 10⁻³ | 19 | 959 | 3074 | 1776 |

### Example 2. Analysis of Purified Anti-FLT3 Murine Antibodies

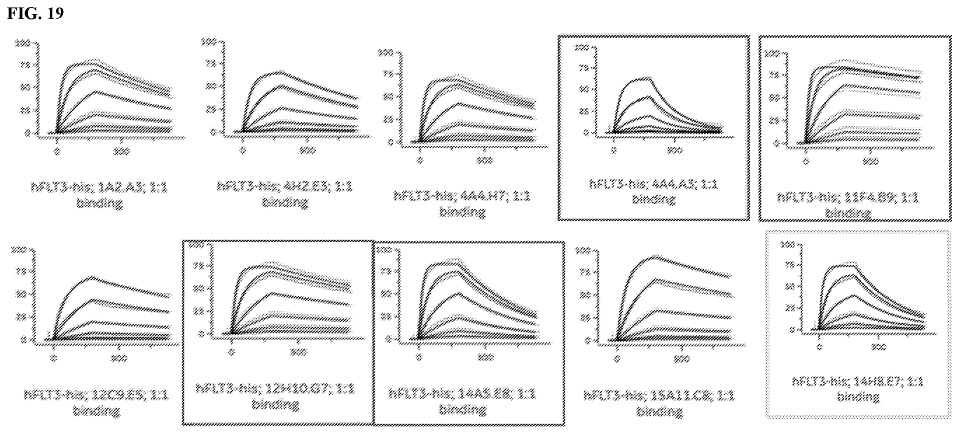

Based on the analysis described in Example 1, eight hybridomas (4A4, 11F4, 12H10, 15A11, 12C09, 1A2, 14A5, 4H2) were selected for subcloning and sequencing. Two subclones from each parental hybridoma were produced and analyzed. Sequences from each hybridoma were determined to be unique. Each subclone was purified from the hybridoma culture, and binding to hFLT3-His was confirmed by SPR as shown in FIG. 19. Kinetic constants and binding affinities of hFLT3 to purified murine subcloned mAbs are shown in Table 13. Binning with reference antibodies was conducted using the method described in Example 1, and four antibodies, namely 4A4.A3, 11F4.B9, 1A2.A3, and 4H2.E3, did not compete with any of the reference antibodies for binding to hFLT3-His.

TABLE 13
Kinetic parameters and affinities of hFLT3
binding to purified murine subclones

| Test articles | kₐ (1/Ms) | k_{d} (1/s) | K_{D}, (nM) |
|---|---|---|---|
| 1A2.A3 | 1.1 × 10⁵ | 8.9 × 10⁻⁴ | 8.5 |
| 4A4.A3 | 1.1 × 10⁵ | 8.2 × 10⁻⁴ | 7.3 |
| 4H2.E3 | 5.7 × 10⁴ | 1.0 × 10⁻³ | 17.6 |
| 11F4.B9 | 1.5 × 10⁵ | 2.5 × 10⁻⁴ | 1.7 |
| 12C9.E5 | 3.4 × 10⁴ | 6.3 × 10⁻⁴ | 18.7 |
| 12H10.G7 | 1.0 × 10⁵ | 5.5 × 10⁻⁴ | 5.4 |
| 14A5.E8 | 1.3 × 10⁵ | 1.9 × 10⁻³ | 15.1 |
| 15A11.C8 | 4.5 × 10⁴ | 4.8 × 10⁻⁴ | 10.5 |

Cell binding of the purified subcloned mAbs was confirmed with isogenic human and cynomolgus monkey FLT3 expressing RMA cell lines. With the exception of 12C9.E5, all clones bound to cell surface expressed human and cynomolgus monkey FLT3 (Table 14). Similarly, all subclones bound with high affinity to SEM (DSMZ catalog number ACC 546), a human ALL cell line reported to express FLT3.

TABLE 14
Cell binding confirmation of purified mouse mAbs to
human and cynomolgus monkey FLT3 RMA cell lines

| | RMA- | RMA- | RMA- | RMA- | | |
|---|---|---|---|---|---|---|
| | hFLT3 | hFLT3 | cFLT3 | cFLT3 | SEM | SEM |
| Test | EC50 | Max | EC50 | Max | EC50 | Max |
| articles | (nM) | MFI | (nM) | MFI | (nM) | MFI |
| 1A2.A3 | 0.80 | 499 | 1.82 | 2834 | 5.47 | 1361 |
| 4A4.A3 | 0.72 | 1021 | 1.07 | 5566 | 3.29 | 2352 |
| 4H2.E3 | 0.66 | 696 | 1.56 | 3454 | 7.57 | 1510 |
| 11F4.B9 | 0.53 | 493 | 1.23 | 2589 | 2.43 | 1141 |
| 12C9.E5 | NB* | NB | NB | NB | NB | NB |
| 12H10.G7 | 0.36 | 1136 | 0.94 | 5262 | 3.20 | 2831 |
| 14A5.E8 | ~2.07 | 415 | 1.25 | 1779 | ~1.07 | 1956 |
| 15A11.C8 | 0.41 | 1406 | 0.82 | 6512 | ~1.13 | 3861 |

### Example 3. Ligand Blocking Properties of Selected Anti-FLT3 Murine Antibodies

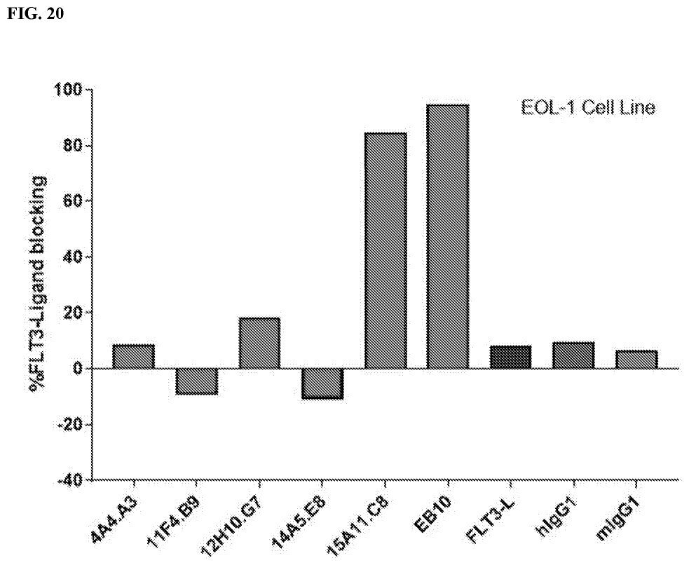

This Example describes experiments designed to characterize the ability of selected anti-FLT3 murine antibodies to block FLT3 interactions with FLT3-ligand. The ability of α-FLT3 mAbs to bind FLT3-expressing EOL-1 cancer cells (DSMZ catalog number ACC 386) was tested before and after the addition of saturating concentrations of soluble FLT3-ligand. For each antibody, its percentage of ligand blocking value was calculated as the decrease in mAb binding signal obtained in the presence of FLT3-ligand relative to that obtained in the absence of FLT3-ligand indicated. Known FLT3-ligand blocker EB10 mAb was used as a positive control. As shown in FIG. 20, the 12H10.G7, 11F4.B9 and 4A4.A3, 14A5.E8 antibodies did not interfere with binding of FLT3 to FLT3-ligand, whereas the 15A11.C8 antibody blocked the binding of FLT3-ligand to FLT3.

### Example 4. Putative Sequence Liability Analysis

This Example describes experiments designed to examine potential sequence liabilities in CDRs (identified under Chothia) of the 12H10.G7, 11F4.B9 and 4A4.A3, 14A5.E8 antibodies. The following potential liabilities were considered: M (potential oxidation site); NG, NS and NT sequence motif (potential deamidation site); DG, DS and DT sequence motif (potential isomerization site); DP sequence motif (potential site for chemical hydrolysis). The results are summarized in Table 15.

TABLE 15
Putative sequence liabilities in the CDRs of selected murine mAbs

| | | location of sequence |
|---|---|---|
| Clone ID | Potential sequence liability motif | liability motif |
| 12H10.G7 | DS (isomerization site) | CDRH3 |
| 14A5.E8 | M (oxidation site) | CDRL1 |
| 11F4.B9 | M (oxidation site), NS (deamidation) | CDRL1 |
| | DP (chemical hydrolysis) | CDRL3 |
| 4A4.A3 | none | |

In addition, a putative sequence liability at M34, which falls within CDRH1 of 12H10.G7 under Kabat, was also identified. Variants of these antibodies were designed to remove the putative sequence liability motifs.

### Example 5. Humanization and Affinity Maturation

Based on the data collected regarding kinetics and affinity for recombinant hFLT3 protein, binding to cell lines expressing human and cynomolgus monkey FLT3, binding to different AML and ALL cancer cells, binning profile, as well as not inhibiting human FLT3-ligand binding, four mouse hybridoma subclones, namely 12H10.G7, 11F4.B9, 4A4.A3 and 14A5.E8, were selected for humanization. Although 4A4.A3 and 14A5.E8 showed slightly lower affinities to hFLT3 than 12H10.G7 and 11F4.B9, these antibodies appeared to bind to a unique epitope (not cross-blocking with reference antibodies) and Domain 1 of FLT3, respectively, and therefore were further analyzed for exploring epitope diversity.

The 12H10.G7 antibody was humanized to create GB94 and GB102 as described supra, which shared the same VH and VL sequences. Back mutations were introduced in the framework regions to create variants GB87 to GB93 and GB95 to GB101.

The 11F4.B9 antibody was humanized to create 1153 and 1154 as described supra, which shared the same VH and VL sequences. Back mutations were introduced in the framework regions to create variants 1151 and 1152. The 1153 antibody was also subject to affinity maturation. Briefly, a library focused on CDRs of the 1553 FLT3 scFv was designed and displayed on the surface of yeast. FACS selection was performed twice by incubating the yeast with biotinylated human FLT3-His antigen. The FACS-enriched output samples were combined with additional CDR mutants to make a second library. Two rounds of additional FACS selection were carried out by titrating with biotinylated human FLT3-His from 100 nM to 1 nM. Sorting was performed at 10 nM, where a clear increase in signal was observed for the library compared to the parent. Sorted yeast clones were plated and screened.

### Example 6. Assessment of TriNKET Binding to Cells Expressed Human Cancer Antigens

Isogenic cell lines ectopically expressing human and cynomolgus monkey FLT3 were used to assess cross-reactivity between human and cynomolgus monkey FLT3. Human cancer cell line RMA expressing hFLT3 or cFLT3 was used to assess tumor antigen binding of FLT3-targeting TriNKET and parental mAb. The human AML cell lines MOLM-13 and MV4-11 and the human ALL cell line REH were used to assess binding ability of the TriNKET or parental mAb. In particular, MOLM-13 cells and MV4-11 cells, which expressed FLT3-T227M and FLT3-ITD, respectively, were used to assess the ability of the FLT3-targeting TriNKET and parental mAb to bind mutant FLT3.

The GB102 monoclonal antibody in the human IgG1 format, also called 1158 mAb, and its corresponding TriNKET F3′-GB102 described supra, also called F3′-1158, were diluted and incubated with the respective cells. The cells were then incubated with a fluorophore conjugated anti-human IgG secondary antibody and were analyzed by flow cytometry. The mean fluorescence intensity (MFI) values were normalized to secondary antibody only controls to obtain fold over background (FOB) values.

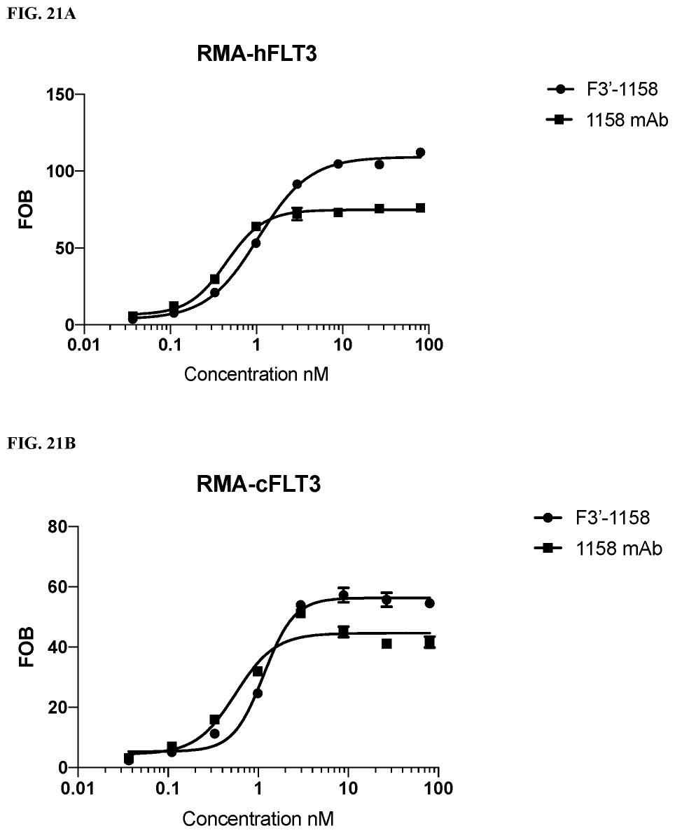

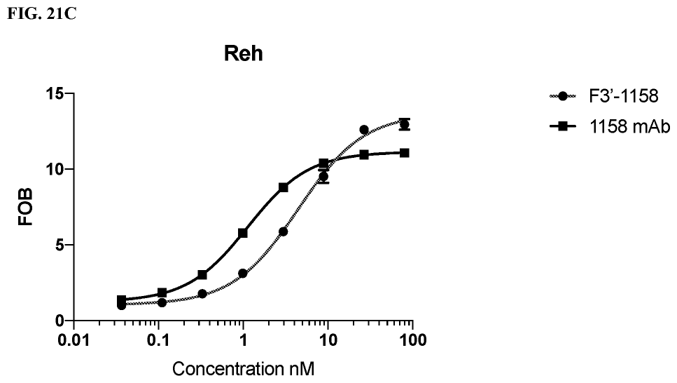

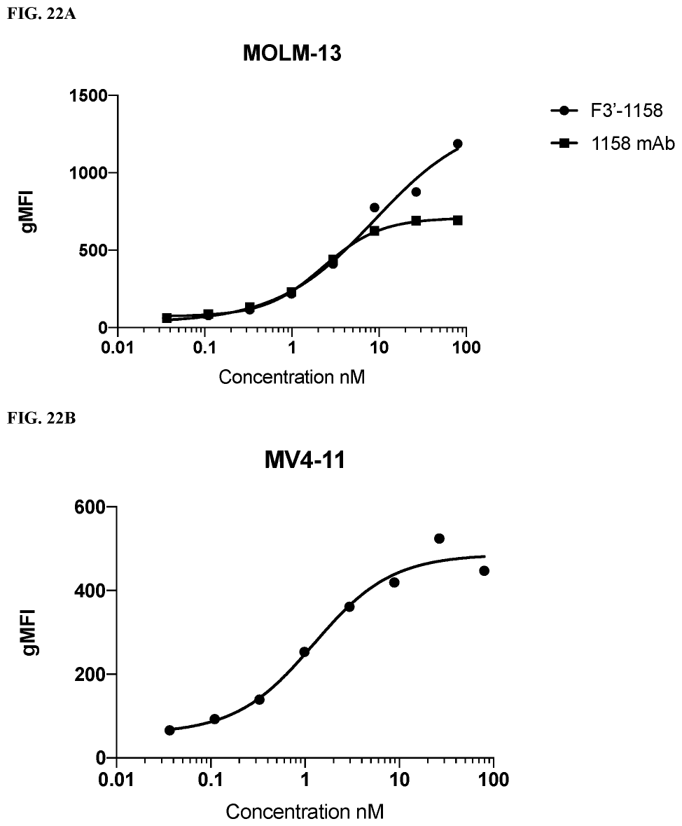

As shown in FIG. 21A and FIG. 21B, F3′-1158 and 1158 mAb each bound RMA cells ectopically expressing human and cynomolgus FLT3 with equivalent potency. As shown in FIG. 21C, F3′-1158 and 1158 mAb bound REH cells, which were human ALL cells. As shown in FIGS. 22A and 22B, F3′-1158 and 1158 mAb each bound MOLM-13 cells and MV4-11 cells, which expressed FLT3-T227M and FLT3-ITD, respectively.

### Example 7. Assessment of TriNKET or mAb Internalization

The EOL-1 human cancer cell line, derived from eosinophilic leukaemia, was used to assess internalization of FLT3 after incubation with F3′-1158 or 1158 mAb. EOL-1 cells in duplicate plates were incubated with F3′-1158, 1158 mAb, or hIgG1 isotype control antibody at 37° C. for two hours. After incubation, the cells were washed and total FLT3 was stained using a non-competing anti-FLT3 antibody. Internalization of FLT3 was calculated as follows:

% internalization=(1−(sample MFI 2 hrs/hIgG1 isotype MFI 2 hrs))×100%

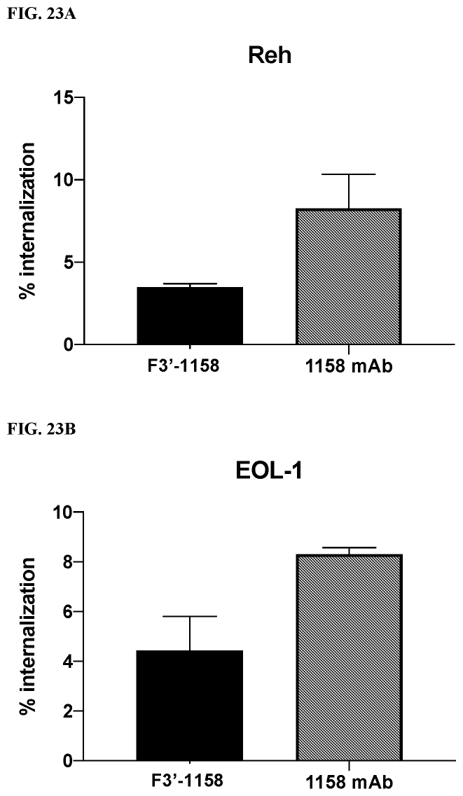

FIGS. 23A-23B show internalization of FLT3 after incubation with F3′-1158 and 1158 mAb in REH (FIG. 23A) and EOL-1 (FIG. 23B). The parental mAb induced less than 10% internalization of FLT3, while FLT3-targeted TriNKET induced less than 5% internalization of FLT3.

### Example 8. Primary Human NK Cell Cytotoxicity Assay

Lysis of target cells was measured by the DELFIA cytotoxicity assay. Briefly, human cancer cell lines expressing FLT3 were harvested from culture, washed with HBS, and resuspended in growth media at 10⁶/mL for labeling with BATDA reagent (Perkin Elmer C136-100). Manufacturer instructions were followed for labeling of the target cells. After labeling, cells were washed three times with HBS, and were resuspended at 0.5-1.0×10⁵/mL in culture media. 100 μl of BATDA labeled cells were added to each well of the 96-well plate. Monoclonal antibodies or TriNKETs against FLT3 were diluted in culture media, and 50 μl of diluted mAb or TriNKET were added to each well.

To prepare NK cells, PBMCs were isolated from human peripheral blood buffy coats using density gradient centrifugation, washed, and prepared for NK cell isolation. NK cells were isolated using a negative selection technique with magnetic beads. Purity of isolated NK cells was typically >90% CD3-CD56+. Isolated NK cells were rested overnight and harvested from culture. The cells were then washed and resuspended at concentrations of 10⁵-2.0×10⁶/mL in culture media for an effector-to-target (E:T) ratio of 5:1. 50 μl of NK cells were added to each well of the plate for a total of 200 μl culture volume. The plate was incubated at 37° C. with 5% CO₂ for 2-3 hours.

After the incubation, the plate was removed from the incubator and the cells were pelleted by centrifugation at 200×g for 5 minutes. 20 μl of culture supernatant were transferred to a clean microplate and 200 μl of room temperature europium solution (Perkin Elmer C135-100) were added to each well. The plate was protected from light and incubated on a plate shaker at 250 rpm for 15 minutes, then read using SpectraMax i3X instruments.

Spontaneous release of substance that can form a fluorescent chelate with europium was measured in target cells incubated in the absence of NK cells. Maximum release of such substance was measured in target cells lysed with 1% Triton-X. % Specific lysis was calculated as follows:

% Specific lysis=((Experimental release−Spontaneous release)/(Maximum release−Spontaneous release))*100%.

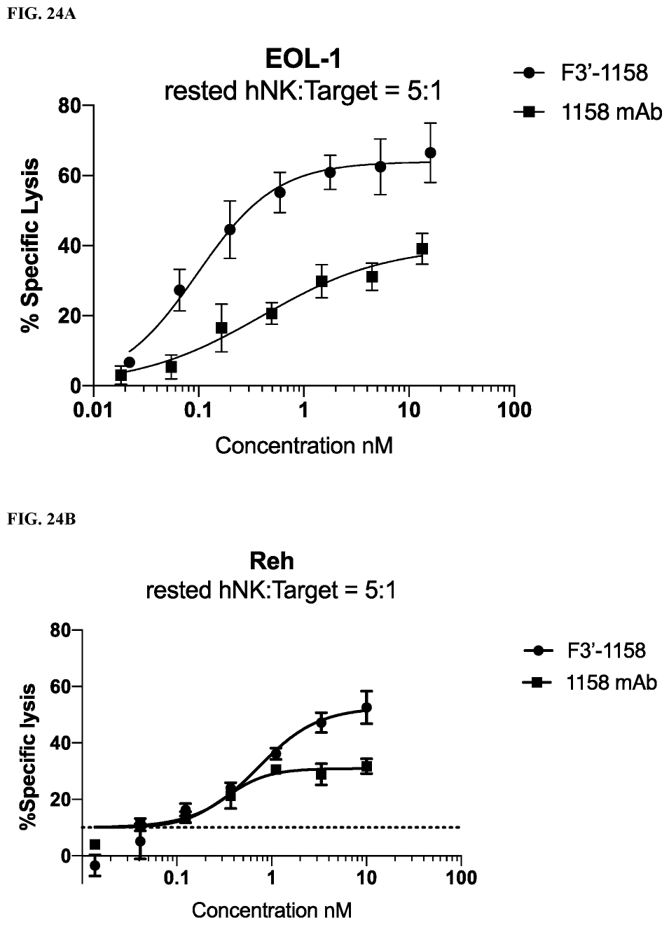

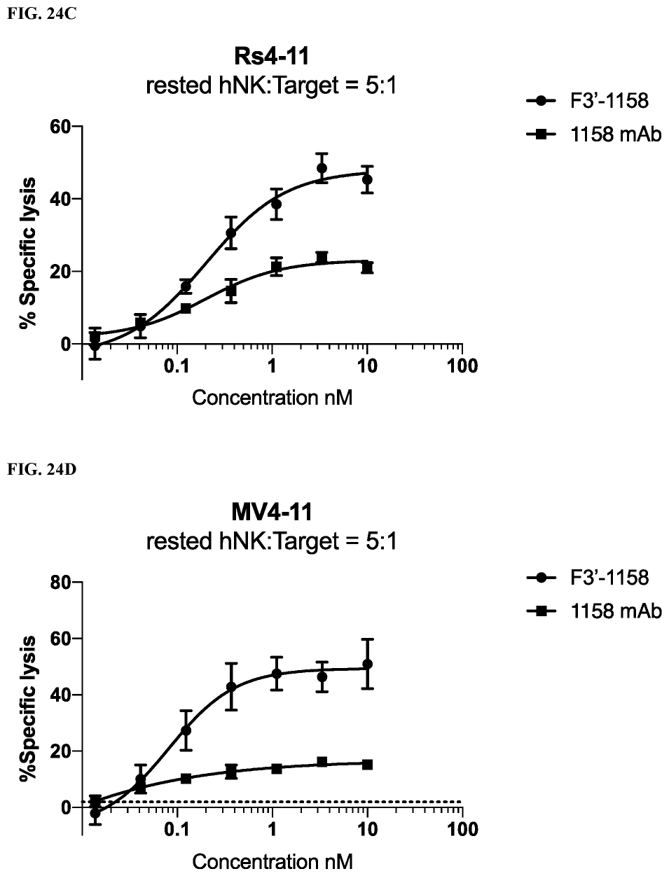

FIGS. 24A-24D show the activity of F3′-1158 or 1158 mAb in enhancing primary NK cell-mediated killing of human AML or ALL cell lines EOL-1 (FIG. 24A), Reh (FIG. 24B), RS4-11 (FIG. 24C), and MV4-11 (FIG. 24D). F3′-1158 demonstrated more potent killing compared to its parental mAb 1158 mAb.

To assess whether the antigen-binding site that binds NKG2D and the effector function of the Fc contributed to the cytotoxicity of F3′-1158, two TriNKET variants were constructed. The first variant contains mutations in the light chain variable domain of the A49 antigen-binding site that binds NKG2D. As a result, this variant does not bind human NKG2D. The amino acid sequence of the mutant light chain variable domain is DIQMTQSPSTLSASVGDRVTITCRASNSIS SWLAWYQQKPGKAPKLLIYEAS STKSGV PSRFSGSGSGTEFTLTISSLQPDDFATYYCQQYDDLPTFGGGTKVEIK (SEQ ID NO:305). The amino acid sequences of this first variant are otherwise identical to those of F3′-1158. The second variant contains mutations in the Fc domain. Specifically, each polypeptide chain of the Fc domain contains L234A/L235A/P329G substitutions (numbered under EU numbering system), which was reported to reduce the binding of the Fc to Fcγ receptors. The amino acid sequences of this second variant are otherwise identical to those of F3′-1158.

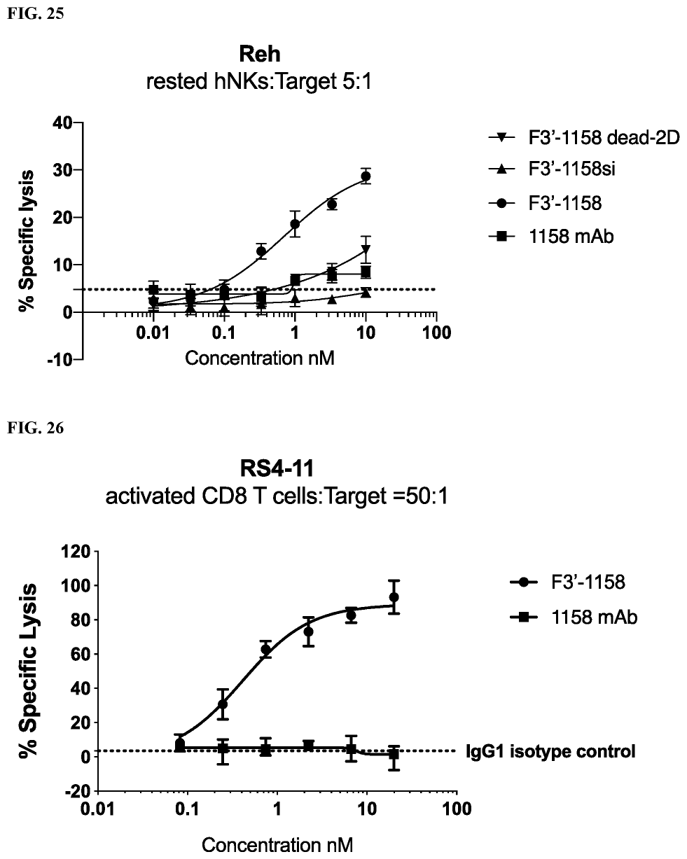

The ability of these variants to induce NK cell-mediated lysis of target cells was assessed using the DELFIA cytotoxicity assay described above. As shown in FIG. 25, the mutations in the NKG2D-targeting domain and in the Fc domain substantially reduced cytotoxicity. This result suggested that binding to NKG2D and binding to Fey receptors both contributed to the cytotoxic activity of F3′-1158.

### Example 9. Primary Human T Cell Cytotoxicity Assay

Lysis of target cells was measured by the DELFIA cytotoxicity assay as described in Example 8, except that CD8+ T cells were used as immune effector cells. CD8+ T cells were prepared as follows: Human PBMCs were isolated from human peripheral blood buffy coats using density gradient centrifugation with Lymphoprep and SepMate 50, according to the manufacturer's instructions. Isolated PBMCs were stimulated with 1 μg/mL ConA (an IL-2 culture supplement) in culture media at 37° C. for 18 hours. ConA was then removed and PBMCs were cultured with 25 units/mL IL-2 at 37° C. for 4 days. CD8+ T cells were purified using a negative selection technique with magnetic beads (EasySep™ Human CD8+ T Cell Isolation Kit), according to the manufacturer's instructions. CD8+ T cells were cultured in media containing 10 ng/mL IL-15 at 37° C. for 6-13 days before using in the cytolysis assay.

As shown in FIG. 26, F3′-1158, but not 1158 mAb, significantly enhanced the ability of CD8 T cell to lyse RS4-11 target cells. F3′-1158 increased CD8+ T cell-mediated lysis of RS4-11 cells in a dose-dependent manner.

### Example 10. Assessment of TriNKET or mAb Binding to Whole Human Blood

The ability of F3′-1158 and 1158 mAb to bind different types of blood cells was assessed. Briefly, human whole blood was incubated with F3′-1158, 1158 mAb, or a human IgG1 isotype control antibody. The blood cells were analyzed by flow cytometry and binding of F3′-1158, 1158 mAb, or the isotype control antibody was detected using a fluorophore conjugated anti-human IgG secondary antibody.

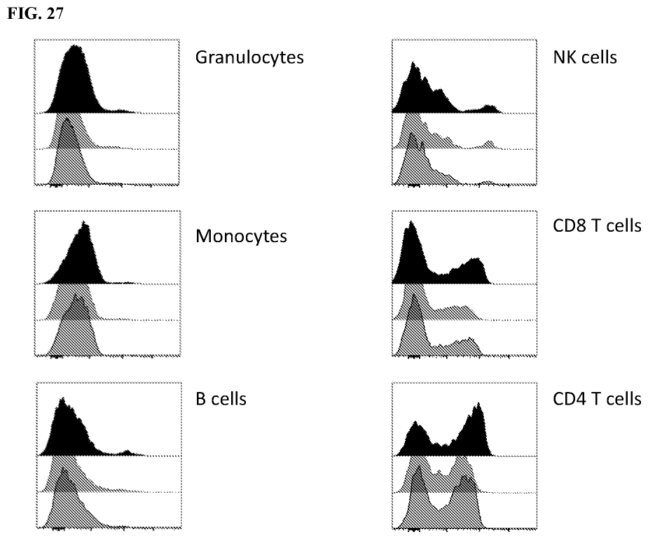

As shown in FIG. 27, F3′-1158 and 1158 mAb showed no significant binding to granulocytes, monocytes, B cells, NK cells, CD8+ T cells, and CD4+ T cells in the blood.

### Example 11. Activation of FLT3 Signaling

Phosphorylation of FLT3, a marker of FLT3 signaling, was measured by pFLT3 ELISA (R&D Systems DYC368). EOL-1 cells were plated in 96 well round bottom plates. F3′-1158, 1158 mAb and/or FLT3L were added. The samples were incubated at room temperature for 5 minutes and were immediately pelleted at 300xg for 5 minutes. The cells were washed twice with PBS. Cell pellets were resuspended in 200 μL of Lysis Buffer #9 and incubated on ice for 15 minutes. The samples were pelleted at 2000 xg for 5 minutes, and the supernatants were transferred to clean test tubes. Protein concentrations were quantified using the BCA total protein assay. Samples were diluted in IC Diluent #12 as appropriate. Lysates were measured according to the manufacturer's instructions. pFLT3 concentration in each sample was determined by interpolation of values from the derived standard curve. Optical density values of the known standards were plotted against their respective concentrations and data was fit to a linear regression model.

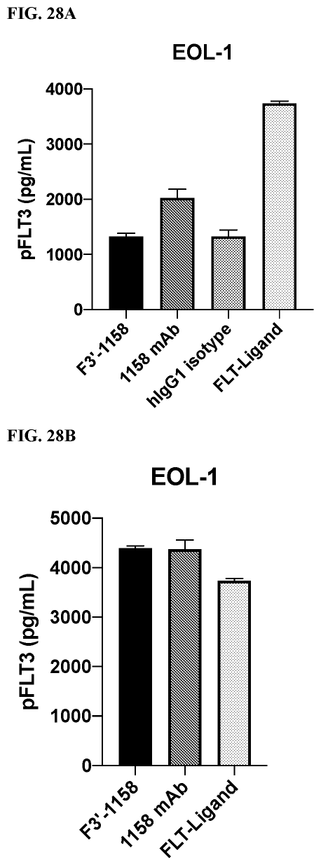

As shown in FIG. 28A, FLT3L led to a 3-fold increase in pFLT3 levels, whereas F3′-1158 and 1158 mAb did not induce significant FLT3 phosphorylation. FIG. 28B shows that when the cells were incubated with F3′-1158 or 1158 mAb in combination with FLT3L, neither F3′-1158 nor 1158 mAb inhibited FLT3L-induced FLT3 phosphorylation. These results were consistent with the observation that 1158 mAb did not compete with FLT3L for binding FLT3.

### INCORPORATION BY REFERENCE

Unless stated to the contrary, the entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A protein comprising:
(a) a first antigen-binding site that binds NKG2D;
(b) a second antigen-binding site that binds FLT3; and
(c) an antibody Fc domain or a portion thereof sufficient to bind CD16, or a third antigen-binding site that binds CD16,
wherein the second antigen-binding site that binds FLT3 comprises:
(i) a heavy chain variable domain (VH) comprising complementarity-determining region 1 (CDR1), complementarity-determining region 2 (CDR2), and complementarity-determining region 3 (CDR3) comprising the amino acid sequences of SEQ ID NOs: 11, 4, and 55, respectively; and a light chain variable domain (VL) comprising CDR1, CDR2, and CDR3 comprising the amino acid sequences of SEQ ID NOs: 6, 7, and 8, respectively;
(ii) a VH comprising CDR1, CDR2, and CDR3 comprising the amino acid sequences of SEQ ID NOs: 59, 63, and 54, respectively; and a VL comprising CDR1, CDR2, and CDR3 comprising the amino acid sequences of SEQ ID NOs: 86, 66, and 67, respectively;
(iii) a VH comprising CDR1, CDR2, and CDR3 comprising the amino acid sequences of SEQ ID NOs: 87, 88, and 89, respectively; and a VL comprising CDR1, CDR2, and CDR3 comprising the amino acid sequences of SEQ ID NOs: 91, 92, and 93, respectively;
(iv) a VH comprising CDR1, CDR2, and CDR3 comprising the amino acid sequences of SEQ ID NOs: 97, 99, and 100, respectively; and a VL comprising CDR1, CDR2, and CDR3 comprising the amino acid sequences of SEQ ID NOs: 101, 102, and 103, respectively;
(v) a VH comprising CDR1, CDR2, and CDR3 comprising the amino acid sequences of SEQ ID NOs: 87, 98, and 89, respectively; and a VL comprising CDR1, CDR2, and CDR3 comprising the amino acid sequences of SEQ ID NOs: 106, 92, and 93, respectively;
(vi) a VH comprising CDR1, CDR2, and CDR3 comprising the amino acid sequences of SEQ ID NOs: 109, 110, and 111, respectively; and a VL comprising CDR1, CDR2, and CDR3 comprising the amino acid sequences of SEQ ID NOs: 112, 113, and 114, respectively;
(vii) a VH comprising CDR1, CDR2, and CDR3 comprising the amino acid sequences of SEQ ID NOs: 117, 118, and 119, respectively; and a VL comprising CDR1, CDR2, and CDR3 comprising the amino acid sequences of SEQ ID NOs: 120, 121, and 122, respectively;
(viii) a VH comprising CDR1, CDR2, and CDR3 comprising the amino acid sequences of SEQ ID NOs: 87, 98, and 89, respectively; and a VL comprising CDR1, CDR2, and CDR3 comprising the amino acid sequences of SEQ ID NOs: 106, 92, and 93, respectively;
(ix) a VH comprising CDR1, CDR2, and CDR3 comprising the amino acid sequences of SEQ ID NOs: 62, 33, and 127, respectively; and a VL comprising CDR1, CDR2, and CDR3 comprising the amino acid sequences of SEQ ID NOs: 128, 129, and 130, respectively; or
(x) a VH comprising CDR1, CDR2, and CDR3 comprising the amino acid sequences of SEQ ID NOs: 132, 133, and 134, respectively; and a VL comprising CDR1, CDR2, and CDR3 comprising the amino acid sequences of SEQ ID NOs: 65, 66, and 46, respectively.

2. The protein of claim 1, wherein the second antigen-binding site that binds FLT3 comprises a VH comprising CDR1, CDR2, and CDR3 comprising the amino acid sequences of SEQ ID NOs: 11, 4, and 55, respectively; and a VL comprising CDR1, CDR2, and CDR3 comprising the amino acid sequences of SEQ ID NOs: 6, 7, and 8, respectively.

3. The protein of claim 2, wherein the second antigen-binding site that binds FLT3 comprises a VH comprising CDR1, CDR2, and CDR3 comprising the amino acid sequences of SEQ ID NOs: 11, 4, and 5, respectively; and a VL comprising CDR1, CDR2, and CDR3 comprising the amino acid sequences of SEQ ID NOs: 6, 7, and 8, respectively.

4. The protein of claim 2, wherein the second antigen-binding site that binds FLT3 comprises a VH comprising CDR1, CDR2, and CDR3 comprising the amino acid sequences of SEQ ID NOs: 11, 4, and 50, respectively; and a VL comprising CDR1, CDR2, and CDR3 comprising the amino acid sequences of SEQ ID NOs: 6, 7, and 8, respectively.

5. The protein of any one of claims 2-4, wherein the VH comprises an amino acid sequence at least 90% identical to SEQ ID NO:37, and the VL comprises an amino acid sequence at least 90% identical to SEQ ID NO:38.

6. The protein of any one of claims 2-5, wherein the VH comprises the amino acid sequence of SEQ ID NO:53, and the VL comprises the amino acid sequence of SEQ ID NO:42.

7. The protein of claim 6, wherein the VH and the VL comprise the amino acid sequences of SEQ ID NOs: 9 and 10; 13 and 10; 17 and 10; 9 and 22; 9 and 26; 9 and 30; 9 and 34; 37 and 38; 41 and 42; 45 and 42; or 49 and 42, respectively.

8. The protein of any one of claims 2-7, wherein the second antigen-binding site is present as a single-chain fragment variable (scFv), and wherein the scFv comprises an amino acid sequence selected from SEQ ID NOs: 3, 12, 15, 16, 19, 20, 23, 24, 27, 28, 31, 32, 35, 36, 39, 40, 43, 44, 47, 48, 51, and 52.

9. The protein of claim 1, wherein the second antigen-binding site that binds FLT3 comprises a VH comprising CDR1, CDR2, and CDR3 comprising the amino acid sequences of SEQ ID NOs: 59, 63, and 54, respectively; and a VL comprising CDR1, CDR2, and CDR3 comprising the amino acid sequences of SEQ ID NOs: 86, 66, and 67, respectively.

10. The protein of claim 9, wherein the second antigen-binding site that binds FLT3 comprises a VH comprising CDR1, CDR2, and CDR3 comprising the amino acid sequences of SEQ ID NOs: 78, 63, 79, respectively; and a VL comprising CDR1, CDR2, and CDR3 comprising the amino acid sequences of SEQ ID NOs: 80, 66, 67, respectively.

11. The protein of claim 9, wherein the second antigen-binding site that binds FLT3 comprises a VH comprising CDR1, CDR2, and CDR3 comprising the amino acid sequences of SEQ ID NOs: 62, 63, 64, respectively; and a VL comprising CDR1, CDR2, and CDR3 comprising the amino acid sequences of SEQ ID NOs: 65, 66, 67, respectively.

12. The protein of any one of claims 9-11, wherein the VH comprises an amino acid sequence at least 90% identical to SEQ ID NO:76, and the VL comprises an amino acid sequence at least 90% identical to SEQ ID NO:77.

13. The protein of any one of claims 9-12, wherein the VH comprises the amino acid sequence of SEQ ID NO:29, and the VL comprises the amino acid sequence of SEQ ID NO:84.

14. The protein of claim 13, wherein the VH and the VL comprise the amino acid sequences of SEQ ID NOs: 68 and 69; 72 and 73; or 76 and 77, respectively.

15. The protein of any one of claims 9-14, wherein second the antigen-binding site is present as an scFv, and wherein the scFv comprises an amino acid sequence selected from SEQ ID NOs: 70, 71, 74, 75, 81, and 82.

16. The protein of claim 1, wherein the second antigen-binding site that binds FLT3 comprises a VH comprising CDR1, CDR2, and CDR3 comprising the amino acid sequences of SEQ ID NOs: 87, 88, and 89, respectively; and a VL comprising CDR1, CDR2, and CDR3 comprising the amino acid sequences of SEQ ID NOs: 91, 92, and 93, respectively.

17. The protein of claim 1, wherein the second antigen-binding site that binds FLT3 comprises a VH comprising CDR1, CDR2, and CDR3 comprising the amino acid sequences of SEQ ID NOs: 97, 99, and 100, respectively; and a VL comprising CDR1, CDR2, and CDR3 comprising the amino acid sequences of SEQ ID NOs: 101, 102, and 103, respectively.

18. The protein of claim 1, wherein the second antigen-binding site that binds FLT3 comprises a VH comprising CDR1, CDR2, and CDR3 comprising the amino acid sequences of SEQ ID NOs: 87, 98, and 89, respectively; and a VL comprising CDR1, CDR2, and CDR3 comprising the amino acid sequences of SEQ ID NOs: 106, 92, and 93, respectively.

19. The protein of claim 1, wherein the second antigen-binding site that binds FLT3 comprises a VH comprising CDR1, CDR2, and CDR3 comprising the amino acid sequences of SEQ ID NOs: 109, 110, and 111, respectively; and a VL comprising CDR1, CDR2, and CDR3 comprising the amino acid sequences of SEQ ID NOs: 112, 113, and 114, respectively.

20. The protein of claim 1, wherein the second antigen-binding site that binds FLT3 comprises a VH comprising CDR1, CDR2, and CDR3 comprising the amino acid sequences of SEQ ID NOs: 117, 118, and 119, respectively; and a VL comprising CDR1, CDR2, and CDR3 comprising the amino acid sequences of SEQ ID NOs: 120, 121, and 122, respectively.

21. The protein of claim 1, wherein the second antigen-binding site that binds FLT3 comprises a VH comprising CDR1, CDR2, and CDR3 comprising the amino acid sequences of SEQ ID NOs: 87, 98, and 89, respectively; and a VL comprising CDR1, CDR2, and CDR3 comprising the amino acid sequences of SEQ ID NOs: 106, 92, and 93, respectively.

22. The protein of claim 1, wherein the second antigen-binding site that binds FLT3 comprises a VH comprising CDR1, CDR2, and CDR3 comprising the amino acid sequences of SEQ ID NOs: 62, 33, and 127, respectively; and a VL comprising CDR1, CDR2, and CDR3 comprising the amino acid sequences of SEQ ID NOs: 128, 129, and 130, respectively.

23. The protein of claim 1, wherein the second antigen-binding site that binds FLT3 comprises a VH comprising CDR1, CDR2, and CDR3 comprising the amino acid sequences of SEQ ID NOs: 132, 133, and 134, respectively; and a VL comprising CDR1, CDR2, and CDR3 comprising the amino acid sequences of SEQ ID NOs: 65, 66, and 46, respectively.

24. The protein of any one of the preceding claims, wherein the second antigen-binding site binds human FLT3 with a dissociation constant (K_{D}) smaller than or equal to 20 nM as measured by surface plasmon resonance (SPR).

25. The protein of any one of claims 2-8, 16, 17, and 21, wherein the second antigen-binding site binds human FLT3 with a K_{D} smaller than or equal to 10 nM as measured by SPR.

26. The antigen-binding site of any one of claims 2-8, wherein the antigen-binding site binds a human FLT3 variant comprising the amino acid sequence of SEQ ID NO:318.

27. The antigen-binding site of any one of claims 2-8, wherein the antigen-binding site binds a human FLT3 variant comprising the amino acid sequence of SEQ ID NO:319.

28. The protein of any one of claims 2-21 and 23-27, wherein the antigen-binding site binds cynomolgus FLT3.

29. The protein of any one of claims 2-20 and 22-28, wherein the antigen-binding site does not compete with FLT3L for binding FLT3.

30. The protein of any one of the preceding claims, wherein the protein comprises an antibody Fc domain or a portion thereof sufficient to bind CD16.

31. The protein of any one of claims 1-30, wherein the first antigen-binding site that binds NKG2D is an Fab fragment, and the second antigen-binding site that binds FLT3 is an scFv.

32. The protein of any one of claims 1-30, wherein the first antigen-binding site that binds NKG2D is an scFv, and the second antigen-binding site that binds FLT3 is an Fab fragment.

33. The protein of any one of the preceding claims, further comprising an additional antigen-binding site that binds FLT3.

34. The protein of any one of claims 1-30 and 32-33, wherein the first antigen-binding site that binds NKG2D is an scFv, and the second and the additional antigen-binding sites that bind FLT3 are each an Fab fragment.

35. The protein of any one of claims 1-30 and 33, wherein the first antigen-binding site that binds NKG2D is an scFv, and the second and the additional antigen-binding sites that bind FLT3 are each an scFv.

36. The protein of claim 31, 32, 34, or 35, wherein the scFv that binds FLT3 and/or the scFv that binds NKG2D comprise a heavy chain variable domain and a light chain variable domain.

37. The protein of claim 36, wherein the scFv is linked to an antibody constant domain or a portion thereof sufficient to bind CD16, via a hinge comprising Ala-Ser or Gly-Ser.

38. The protein of claim 37, wherein the hinge further comprises amino acid sequence Thr-Lys-Gly.

39. The protein of any one of claims 36-38, wherein the heavy chain variable domain of the scFv forms a disulfide bridge with the light chain variable domain of the scFv.

40. The protein of claim 39, wherein the disulfide bridge is formed between C44 of the heavy chain variable domain and C100 of the light chain variable domain, numbered under the Kabat numbering scheme.

41. The protein of any one of claims 36-40, wherein the heavy chain variable domain of the scFv is linked to the light chain variable domain of the scFv via a flexible linker.

42. The protein of claim 41, wherein the flexible linker comprises (G₄S)₄.

43. The protein of any one of claims 36-42, wherein within the scFv the heavy chain variable domain is positioned at the C-terminus of the light chain variable domain.

44. The protein of any one of claims 36-42, wherein within the scFv the heavy chain variable domain is positioned at the N-terminus of the light chain variable domain.

45. The protein of any one of claims 31-34 and 36-44, wherein the Fab is not positioned between an antigen-binding site and the Fc or the portion thereof.

46. The protein according any one of the preceding claims, wherein the first antigen-binding site that binds NKG2D comprises a VH comprising CDR1, CDR2, and CDR3 comprising the amino acid sequences of SEQ ID NOs: 240 or 241, 242, and 270 or 271, respectively; and a VL comprising CDR1, CDR2, and CDR3 comprising the amino acid sequences of SEQ ID NOs: 276, 236, and 245, respectively.

47. The protein of claim 46, wherein the first antigen-binding site that binds NKG2D comprises:
(i) a VH comprising CDR1, CDR2, and CDR3 comprising the amino acid sequences of SEQ ID NOs: 240 or 241, 242, and 255 or 256, respectively; and a VL comprising CDR1, CDR2, and CDR3 comprising the amino acid sequences of SEQ ID NOs:276, 236, and 245, respectively; or
(ii) a VH comprising CDR1, CDR2, and CDR3 comprising the amino acid sequences of SEQ ID NOs: 240 or 241, 242, and 243 or 244, respectively; and a VL comprising CDR1, CDR2, and CDR3 comprising the amino acid sequences of SEQ ID NOs: 276, 236, and 245, respectively.

48. The protein of claim 46 or 47, wherein the VH of the first antigen-binding site comprises an amino acid sequence at least 90% identical to SEQ ID NO:254, and the VL of the first antigen-binding site comprises an amino acid sequence at least 90% identical to SEQ ID NO:239.

49. The protein of any one of claims 46-48, wherein the VH of the first antigen-binding site comprises the amino acid sequence of SEQ ID NO:254, and the VL of the first antigen-binding site comprises the amino acid sequence of SEQ ID NO:239.

50. The protein of any one of the preceding claims, wherein the antibody Fc domain is a human IgG1 antibody Fc domain.

51. The protein of claim 50, wherein the antibody Fc domain or the portion thereof comprises an amino acid sequence at least 90% identical to SEQ ID NO:136.

52. The protein of claim 50 or 51, wherein at least one polypeptide chain of the antibody Fc domain comprises one or more mutations, relative to SEQ ID NO:136, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system.

53. The protein of any one of claims 50-52, wherein at least one polypeptide chain of the antibody Fc domain comprises one or more mutations, relative to SEQ ID NO:136, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system.

54. The protein of any one of claims 50-53, wherein one polypeptide chain of the antibody heavy chain constant region comprises one or more mutations, relative to SEQ ID NO:136, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:136, at one or more positions selected from Q347, Y349, L351, S354, E356, E357, S364, T366, L368, K370, N390, K392, T394, D399, D401, F405, Y407, K409, T411, and K439, numbered according to the EU numbering system.

55. The protein of claim 54, wherein one polypeptide chain of the antibody heavy chain constant region comprises K360E and K409W substitutions relative to SEQ ID NO:136; and the other polypeptide chain of the antibody heavy chain constant region comprises Q347R, D399V and F405T substitutions relative to SEQ ID NO:136, numbered according to the EU numbering system.

56. The protein of claim 54 or 55, wherein one polypeptide chain of the antibody heavy chain constant region comprises a Y349C substitution relative to SEQ ID NO:136; and the other polypeptide chain of the antibody heavy chain constant region comprises an S354C substitution relative to SEQ ID NO:136, numbered according to the EU numbering system.

57. A protein comprising:
(a) a first polypeptide comprising the amino acid sequence of SEQ ID NO:278;
(b) a second polypeptide comprising the amino acid sequence of SEQ ID NO:279; and
(c) a third polypeptide comprising an amino acid sequence selected from SEQ ID NO: 277, 283, 284, 285, 286, 287, 288, and 289.

58. A protein comprising:
(a) a first polypeptide comprising the amino acid sequence of SEQ ID NO:280;
(b) a second polypeptide comprising the amino acid sequence of SEQ ID NO:281; and
(c) a third polypeptide comprising the amino acid sequence of SEQ ID NO:282.

59. A pharmaceutical composition comprising a protein of any one of the preceding claims and a pharmaceutically acceptable carrier.

60. A cell comprising one or more nucleic acids encoding a protein of any one of claims 1-58.

61. A method of enhancing tumor cell death, the method comprising exposing the tumor cell and a natural killer cell to an effective amount of the protein of any one of claims 1-58 or the pharmaceutical composition of claim 59.

62. A method of treating cancer, the method comprising administering an effective amount of the protein of any one of claims 1-58 or the pharmaceutical composition of claim 59 to a patient in need thereof.

63. The method of claim 62, wherein the cancer is a hematologic malignancy.

64. The method of claim 63, wherein the hematologic malignancy is leukemia.

65. The method of claim 63 or 64, wherein selected from the group consisting of acute myeloid leukemia (AML), acute lymphoblastic leukemia (ALL), myelodysplasia, acute T-lymphoblastic leukemia, and acute promyelocytic leukemia.

66. The method of any one of claims 62-65, wherein the cancer expresses FLT3.
